SEQUENCE LISTING (274 sequences)

SEQ ID NO: 1 - DNA, 423 bp, Homo sapiens
  source 1..423: mol_type = genomic DNA
aaaatcagaacaaactgaagatatgggccagaacttgtataaagtgtgaaaagcagtcaataaagaaagttagaaatactttgcattttttttttaatcacaggacctgagttaagccaagaatacagtagaaattttatcaagtagagataagctctcagtaaaggataaaagtgggcctaagtcccttcagtttcactggaagtaggacccttacattttataattatattttcatacataagctactggacaatgaagtaaatagcaatcagtgaaagagccacatatgaccaacttagatttccttgagtaaagtctgtcaagggtaaagctgtgaaagtttataagaaaaaagaatggggaattatttggaagaccatttgagttttgtacacaagaatttaatgtttgcacacttga

SEQ ID NO: 2 - DNA, 588 bp, Homo sapiens
  source 1..588: mol_type = genomic DNA
ataaaccaaaagtaagattctaagcctccccctcatttaaaggacttcctcctcagccagggctcttaaaatttaacctgaaagactggttcaggccatgaagggaaatgggggtcagacatgcttcattatgcctctctggcattaatatcaacacaggctttcagtctaataagaaacattttacagcctgtttctctgtgaagcctgctagctgaaagcttcatctgcaggataaaactttggtctccacaacctcttatcacaactcaaacattccttcctattgatctccggtctttagacaaactcaaccaattgtcaaccagaaaacgtttcaatttacctgtagcctggaaacgcccactttgagttgtcccgcctttctagaccaaaccgatgtatttctcaagtgtacttaattgatgtctcatgctccctaagtgtataaaatcaagctacaccccaaccaccttgggcacatgtcatcaggacttcctgaggctgtgtcacaggcatacgtcctcaaccttggcaaaataaactttctaaattaactgacaccagtctcagattttccgggttcac

SEQ ID NO: 3 - DNA, 2629 bp, Homo sapiens
  source 1..2629: mol_type = genomic DNA
aacccctgtaaagtgactctgaagaagacgagaagccctgctccagtcacacccagaagctaactggtccacgcatggctgaagcatgagaaaactcttcatgggactcattttccttaaaatttggacttgtatagtaagtacttcaactgaccttcctcagactgaggactgttcccagtgtatatatcaagtcacagaggtaggacaaaaagttgctacagtcttattttatgcttattgtaagtgtactgggactgtaaaaagaacttgtttgtataatgctattctatacaaggtatgtagcccaggaaatgaccaacctgatgtgtgttatgacccatctgagcctcccatgaccacagttttaaaaataagattaagtactgaggactggtgggggctcataaatgatatgagtaaagtgttagccaaaacagaaggaaagaaaagggattcccaaacaagtcaccttgaaatttgatgcctgtgctgtcattaatagtaataagttagaaacatgatgtggttctcttaattaggaaagaggctgtatggcagaaaataagtacatttgtcatgaattaggactgtgtggaaataaatgtagatactggtcttgtgtcatttaggctatgtggataaaaaataaaaagaatcctgtccaccttcagaaagggaaagtggcccttcctgtaccagtggtcagtgtaaccccttagaactagtaataaccaatccccttgatccttgctgaaaaaatggggagcatgtaaccctagaaattgatggggctggactggatcctcgagtaaatatcgtggtttgaggagaagtttataaatgctctcctgagccagtatttcaaatcttctatgatgaactaaatgtgccagtaccagaaattccaggaaaaacaagaaatttgtttttgcaataagccgagcatgtagcccagtctctcaatgtcacttcatgttatgtatgtggaggaactgtaatgggagatcaatggccatgggaagcccgagaattagtacctacagacccagttcctgataaattcccagctcaaaagactcaccctgataacttctaggtcctaaaagcctcaatcattagacaatactgtatagcaagagtggggaaggacttcacccttcctgtgggaagactcagctgccttgggcaaaaactgtataatagtactacaaaaacagccacctggtggagttcaaaccacactaagaaaaatccatttagttaattcccaaagttgcaaactgtgtggacccacccagagtcccaccaggactggacagcccccactggattatactggatatgtgtgcatagagcttacaccaaattacccggccagtgggcaggtagttgtgttattggcactattaaaccatctttcttcctactgcccataaagacaggtgaactcctgggcttccctgtctatgcttcctgcaaaaagagaagcatagctataggaaattggaaagatgatgaatggccccctgaaagaataatataatattatgggcctgctacttgggcacaagatggctcatagggattccagactcccatttacatactcaaccaaatcatatggttacaagctatcttagaaataatcactaataagactggcagagccttgactattctggcccagcaagaaactcagatgagaaatgctatctatcaaaatagattggctcttgacaacttgctagcagctgaaggaggggtctgtaggaaatttaaccttactaattgctgtctacacatagatgatcagggcaagcagttgaagacatagttagaaatataacaaaattggcacatgtgcccatgcaagtgtggcatggatttgatcctggggccatgtttgaaaaatggttcccagtgctaagaagatttaaaactcttataataggagttataatagtaatagaaacctgcttactgctcccttgtttgctacccatacttcttcaaatgataaaaagcttcatcactaccttagtttaccaaaatgcttcagcacaagtgtactatatgaatcactatcgatctgtcttacaagaagacatgggtagtgagaatgaaagtgagaactcccactaatgagtgagattctcaaagagggggaataagggaggagaccacccctcatattgtcttatgcccaatttctgcctccaaagaagaagaagtaaaaactaaaaggcagaaatgaaatccacaggcagatagcctggtgccgtgccctgggcctggttaaagatcaacccctgacctaatcagttacgttatctatagattccagacattgtatggaaaagcactgtgaaaatccctgtcctgttctgttccgttctgattactggtgcatgcagcccccagtcatgtaccgcctgcttgctcaatcaatcacgaccctttcatgtggacccccttagagctgtaagcccttaaaagggacaggaattgctcactcggggagctcagtttttggagacgtgagtctgctgatgctcccagtggagtaaagctcttccttctacaactcggtgtctgagtggttttgtctgcagctcatcctgctaca

SEQ ID NO: 4 - DNA, 379 bp, Homo sapiens
  source 1..379: mol_type = genomic DNA
tacatgtctcagactcctctgcagccagaggtctggctgtaatttagattctgctaagaaagggaggtgggatccgtcttcccgcagctctggcagctggccatccctgatggatgtttgtggttgcaggaatctaacctaggtctcgactccagttttgtccatgtggggcagtgatggcaggggtagtggtggcattagcagtagcagtggctgctctctggcctctgattcggctacagtggtctaatattgaaaccaatagtccagtggtggcctctgccttctgctcctccagcctttccagtgatttagataaaatccctttctgcctaagattcctagagtgctttctgttttctgggctgaaccttgacta

SEQ ID NO: 5 - DNA, 380 bp, Homo sapiens
  source 1..380: mol_type = genomic DNA
taatcacccctgtggcagctttttgaagtaaggagttcagtatggttagtgcagagtaaagtttggaggaggatagcagtgaacagtagaatggagggaccaggtggagataattgaatcatggggcagttcgcatcatagtgagttagttcttataagatgtgatggttttataaagggcttcccctttctctgggcactcatttccctctcctgctgccacgtgaagaaggacatgtttgcttccctttctgacatgattgtaagcttcctgaggcctcctcagccctgtggaactgtgagtcaattaaacctctttcctttatacattacccagtctcaggcagttctttatagcagtgtgagaacagactaataca

SEQ ID NO: 6 - DNA, 466 bp, Homo sapiens
  source 1..466: mol_type = genomic DNA
agattagcatttgaattagtaaactgcataaaaaagatccaacatgagctgacatcatccaatccattgagggcccaaatagagcaaaaaggcagaggaagagcaaattctctattcttgagctggagtatccatcttcttctgctctcagatatctgagctcctggttctcaaacattcggattctgggacttacacctccagctctctcagttcccacagttcccaggactttgatttcagactgggagttccaccactggctccaccagttctcaggcatttggattcagactgaattacaccaccagctttcctggctttacatctggcaggtggcagatagtgagacttctcgccctccaacaccacatgagccacctcccataacaaatttccttagtttttttttaattttattttttttatgttggttcagtttctctgaagaacgctgactaataca

SEQ ID NO: 7 - DNA, 1307 bp, Homo sapiens
  source 1..1307: mol_type = genomic DNA
ttatagtagatagtcaggcagacaggagcaggccaagagaggcccccactaccaggaatgtcaggcaaccatcaggtgatggtcagccaagttatttttgttgtttttctttttttttgagatggagtctcactctgttgcccaggttggagtacagtggcatgacctcggctcactgcaacctctgctacccgggttcaagagattctcctgcctcaaactcccaagtagctgggattacagtctcccacccgccaccacgcccagctaattgttgtatttttagtagagacggggttttgccacgttggccaggctggtctcaaactcctgacctcaggtgatccgcctgcctcagcctcccaaagtgctgggattacaggcatgagccactgcacccagccggccagccagttgttaagctgactctctaaagtaataattggttgcagccagtgccagggaaaggcagtctcccaatagatagaaacacctgaaactggtgatcagcagcttctgataagatctcaggatttgggcaagaaagctcaagcatgtacattaaggggcaaaatggcagagtttaactgatacatgaccttacaggaacattcgactggtaagggaagaacgcctcaagtaagtatatgcacaactccagtaaacacactgggcttgcggcccctcccaagtgctggccggccactgcgcatgcagacagcccaccccaagggaatcattgactccagaagattgccaacatataaaaccccaagtcaaaggccaaaccatgcacccaatctcccaagttgctcacttggccttcttccaagtgtacttgacttcctttcattcctgctctaattttttttttttttttaacacagagtttcattcttgttgcccaggctggagtgcaacggcacgatctcggctcactcaacctctgcctcctgggttccagtgattctcctgcctcagcctcccgagtagctgggattataggcatatgccaccatgcccggctaattttgtatttttagtagagacggggtttctccatgttggtcaggctggtcttgaatttctgacctcaggtgatccgcccgccttggcctcccaaagtgctgggattacaggtgtgacccaccgtgccaggcctctaatactttttaataaactctcactcccgctctaaaacttgtctcagtctctctgccttatgccccttggttgaatttcttctgaggaggcaagaattgaggttggggcaaccagtacagattcactgctgctaacagttgtag

SEQ ID NO: 8 - DNA, 327 bp, Homo sapiens
  source 1..327: mol_type = genomic DNA
tgtcagagactggccagatactcaccaattctgcctctctttcctgcatccataggaaaactacctttcccagcctcccttgcagttaggttgaggccacatgcctgggatctgaccaatggaatgtaagtagcagggatatccgccatttccacacttggccaaaaacccctgtgagattctctttttgctgcatccccttacctgtgtgggtgaaaagggaagaccccaagatgatggactctggtgatgggaggatcctgaatcactgccatcactttgggaaagacttgccagaaagcagcctctcgttggactttgcatgaa

SEQ ID NO: 9 - DNA, 2948 bp, Homo sapiens
  source 1..2948: mol_type = genomic DNA
gtaaattggtaccagaagtggggtgctactgtaaagatacacaaaatgtggaagtgactttggaactgcatacaggcagaggttggaacagtttggagggcagtttggaacagttggaacagtttgaagaagataagaaaatatggaaaagtttggaacttcctaaagacttgttaaatggctttaaccaaaatgctgataatgttatggacaataaagtccaggctgaggtggtctcagatggagatgaggaacttttggggagctggagcaaaagtgaatcttgttatgtttagcaaagagattagttgcattttgcccttgtcctagagatctatggaactttgaacttgagagacgattatctggaagaagaaatttctaaagagcaaatcattcaagaggtgacagagcataagagtttggaaaattcacagcttgagaatgtggtaggagagaaaaatccatgttcgtgggaatggggggcatggaattaaagctggttgcagaaatttgcataagaaatgaggactcaaatgttaattgccaagacaaggggaaaaatgcctctagggtatgtcagagatcttcatgagagaccctcccatcacaggcccagaggcctaagaggaaaaaatgatttcctgggatgggcccagggtccctttctgtgtgcagcctagggatttggtgccctgcatcctacccctccagccattgctaaaacaggccaagataccaggtcaggctgtggcttcagagggtgcaagccccaaaccttggcagcttccacatggtgttgaggctgcaggtgcacaaaagtcaagaattgaggtttgggaacctacgcctaggtttcagaggatgtatggaaatgctggatgtccaggcaaaagtttgctgcaggggtggaaccctcatgtagaatctttgctagggtagtgtggaagggaaatgtggtgttggggctcccatacagagtccccactggagtacagctagtggagttaagagaagaaagccaccatcctccaggccccaggatgatagattcactgacagcttgcaccatgcacttgaaaaagcccagaaactcaacaccagcccatgaaagaaaccaggaagaaagatgtaccctgcaaagccacaggagcatagctgcccaaggccatgggatcctacctcttgcagcagcgtgacctggatatgagacatggagttgaaggagactattttggaactttaaggtttaatgactgccctattaaatttagaacttgcatggggccagtagcccctttgttttagtcaatttctcccatttggaaggggtgtatttactcaatgcctgtactcccattgtatctaggaagtaactaaattgctcttgaatttactgactcataggaagggaccttccttgtctcagatgagactttggacttggattttagagttaatgctggaatgagtgaagactttgggggactgttagaagggcatgattgtgttttgaaatgtgagagcatgagatttgggagaggccaggggtggaatgatatgatttggctataccccaacccaaatcatcttgaattgtagttcccataagccccatgtgtcataggagggacctggtggaaggtaatttaatcatgggggtgattaccctcatgctgtgctcctgatagtgagtgagttctcataagatctgatgtttttataaggggcttttcccctttttgctcagccttctccttgatgccaccatgtgaagaaggatatgttttcttccccttccactatgattgtaagcttcctgaggcctccccagccatacagaactgtgaatcaataaagtctctttcctttataaattacccagtctcaagtatgcctttattagcagcatgagaatggactaatacacattctttgaaaacatggatgtcgaatctaccacaaaacacaagagttaaggagcaccaacttcatgttttctggttgcaatacaatctctattttatactaatacttcaacatagaaggtgtgatacatgtgtatatgtgggtgtcagcttcaattgacatgcaaaaccctggtgagtcattaacagttcatttattttcctgttttagagttaatataataaaatgcataggctatacaatatgtgctcctttatgtctgggttattttgacatgattgttataaatgcattcatgctgtagcagtgatgtgttcttttctcattgtaatgtagtatctaatcgtgttaccatttcgcaatttatccattttataaaaggatgcatgttggggatttggtttattaaacaaatgtctgttggatattggcttatttctaggttttcaggttttcgcttgtcttaaaaagcttctataaacacatatgactgtgtttggatggacatgtgctctctggaagtattaccatttgaagaaacttaatgtgaattacacactctccagaggtgggaggttgataattgaaagtccacaccacccccctcctcaggcagctagaagctttggatatctaatcatggtttaccattagaggctttgttgattaccataatgcttgttggaaatggtccttgatgggataaataagtggaattcaaggtttgtggagttccaagtgccaaaataggcactcctattgttccataaacacagtataagaacccagcagtgccaacaaccaatgccctttggaataaagtctgtgtttgaaactgtcttgagtttggacaatcatagcattttgtcatttaaactcaactaggttcagtgaatcctacatgtagaacattgtcatgtctccctagagtgaaacattcatgaaaagtgctcttcagggcaggcagctgggagcttggcaactttgcacaactggctgcatggctgtttctgtgcctg

SEQ ID NO: 10 - DNA, 356 bp, Homo sapiens
  source 1..356: mol_type = genomic DNA
gtgatagtcattcgtagtgctgctcaccaaataggcccagttctccctattctgagcacatggtgtatcacatgtcctcgtccccactgtggggactcttcctagctcatgagttatatgtgtggcccttctacatgaaagttgtaacagccatgagagaccctccaaagcaggcttcctgctctagcaaccaaccaataacatttgaaatagtgactgtgactgctccttcagcctaagtccctaactgtctgcaaacccacaatggacctgttgcatgtgtaagaaataagcccttgttattttaagccactgagatttaggagctatttgttacagcagcataaccttgactg

SEQ ID NO: 11 - DNA, 497 bp, Homo sapiens
  source 1..497: mol_type = genomic DNA
gttgggtggctgcatcctatggtgtttctacatgtttcatgatagcttttatcttgaataatcttttccggcatgttggtatagtgaacagccttggaaaataaagatagtgtttcctattgtgtcagaaggcaaattttttcctgaccaggcttataaagataacatcttccgctggagcaaaggttgggcagatttgttagcagtcccctcataaaaatcaaggtttttcaagctcaaagtccttcagctatgacatagatctactgtgtatatgacatccacctggccctgccatcatcatccccatgagactaggagggacaagaaccagcgtgaacataaagctcatagttcctgcagtgctgtgaataataaagtcctttgtctctgacccaggagcattgtgtcttctgccagcatctatgaaactatggcagactaatttgttagcttgcaagtagcataaaatctcagacccttcttagttcctgaca

SEQ ID NO: 12 - DNA, 532 bp, Homo sapiens
  source 1..532: mol_type = genomic DNA
tggatttctgtagtttctgcctgcctgtgatacctccctctttcttctggtaacaaactgggctgtccacttgggaattgttgattggtgagttgtcaaatgcagggcccaccactctcctcctctgggttcacgatccaggctagcttcatacaatggtatatgcaatgattagttcagaattttgtgtgtgtgtgtgagtgtgtgtgtgtgtgtgtgtgtgtgtgtgcgtgtgtatgtgatgtttaatgggggaccagaatcctttcttggagattaccatggttgcttggagagagaatcttctttttttgagagtgtgtgtgctaaggatcacaggaacctggatgtgttgagagacagagactgcaagagtaatgacatgacattgtctgaactttgggaaacaattatgtttggagccagagctaccccttggactttccagttaatcgaatcaatccattgtctttttaacctagactagttccaactgggattctgtcactagtatctgcatggtcctaatttc

SEQ ID NO: 13 - DNA, 519 bp, Homo sapiens
  source 1..519: mol_type = genomic DNA
ctctggatgagggtatggtgaatttaaaagatggttgcaaattctttgacatttctccaatggagaggtgggtctgtgtctccttccttgaacctgtgtggatttctgactacagtggaaatgagctatgtgacttccaaggctgggacatacacagccatgcagcttctgtcttgctggccagaacactcacaccagagacttgaggtgcctcgtaagaggtccaatgaccaggccatggtgctggagacatcatgtgtagtctctctggtcaacagtcccagctgagcccagccttccagctctctttgccaagtgaacaacgatcttacaagtggacccttcagccccagctgttccaactcccagttattccagtcacctcgagtcattccagtcatcctagccgtcgtagagcagagaattgcccttctgactccttgacagtggcccaaaaaatggttgttgttttatgctactaagttttgaggtggtttgttatgtagcgttcaataacta

SEQ ID NO: 14 - DNA, 545 bp, Homo sapiens
  source 1..545: mol_type = genomic DNA
cccctctcaaagatgcacacatcctcatcccaagatctgtgaatatggcacatggtcaaagacacttagcagataaatgaaggatgatgagatggggagattattgtggtttatccaggtgggtccaaaataaccatacaagtgagacagggaggcggggaagtcagtgaggatggaagcagagcacagagaggtttcaagatgctgtactgctcgctttgaagatggaggaaggggccaaagttaaggatgcaggcggcctctagtaattggaaaaggtgagggaacagacttttccctggagctgccaggaagaacacagccaagtggacacgttgttgttttatgccactaagttaacaatggagttgtcatctctgttgcctaggccagcgtgcagtggtgtgatcagagctcactgcagcctcaacctcctgggctcaagcgatcctcctgcctcagcctcttgagtaagtgggaccaagacacaaaccaacatgcctggcctgtgggaatttgtcacagcatcaacaggaaactgacac

SEQ ID NO: 15 - DNA, 261 bp, Homo sapiens
  source 1..261: mol_type = genomic DNA
tctctaatggagctgtaacactcaccactgaggtccatggcttctttccttgaagcctgtgaaaccacgaacccttcaatcaagaaaagacctttgatcaggagaagacttctcgtctcatttctggggacacattcaggatttctccaaagcagtgagtaacattgaacccctcttgcttgctattctgttctatcttctcattagaaattggaagaaaacaccaggcacctgtcagccatttaaatgtgacaagcggcc

SEQ ID NO: 16 - DNA, 524 bp, Homo sapiens
  source 1..524: mol_type = genomic DNA
tgtggcagacatggctgcttgcctacccaatacccactctccctttgttttcctactgacaggtcctgggtttcattcagggcagtaaatgtgcttgttcaaaatatttaactctccaggctcccttgcagatggtggaggtcacatgactctgttgtggacagtaagatataaccaaagtctgctggatggagcttctggaaaagttactacttccctggaaaaaagggagcatgcatttaccattcaccatacaccttctcctctttgcctttctccccgtctccctgcctgccatggagatatgatgcctaaaggtggagtagccaactgacaactgttaggacagaagccacaggctttttccatcggttagatgaagcctgggttcttgatgtcttctttcaccatcccctggcacccgcctatacatttattacttgaaacagactgacctttatttggttaggccactgtggtcaggtttctgcaacatggggtcacatgccttcccaactgacaca

SEQ ID NO: 17 - DNA, 460 bp, Homo sapiens
  source 1..460: mol_type = genomic DNA
ggtgaattgcagggatggcctgggtcttcatccctcgtctccatgccatttgccacacacatttgcagttcttgtcactaaacaagcacggttaatttccctcccctttgagtccgggctgtcctggacactggcttcggcccatgtcatgtggcagaagggacagtgtgccagctccaagccttggcctcaagagacctcgtgcatttcacttgcccccttgagcttctgttgttgtcatgagaagaaaatgcccagggtatccggctggagacacaggatgaggtgacacatggatctgagccaagggcagctgagatccgttgacgccagcccactccgacatgtgggtgaactcagccaagatcagccaattctcagccatgtgttttgtgaactcaaatcagatgttattgttgttttggggctgtttgttatgtaggaatagctagctgataca

SEQ ID NO: 18 - DNA, 261 bp, Homo sapiens
  source 1..261: mol_type = genomic DNA
gtatgtgaagttttttttggtttgttttttgttgtttggttttttaatgttccttttccatcttctgcatgctggggcaccagccgctgccccagaatgggttgtcctgggaaacaggtcatgcagggctaccgatgtttagtctggaaaagacatgcagagataaggaagatgttgaggcaggacagaaccaggctcctgttagtccaacaaaagtttactgagcagtgactttgtgtcaggcacctgggaattgtgccg

SEQ ID NO: 19 - DNA, 420 bp, Homo sapiens
  source 1..420: mol_type = genomic DNA
tgctatagtttggatatttaactctccaaaccttatgttgaaattcgacccccagtgttggagctcgggactagtaggaggtgtttgggtcatagggatgatcctgcacgaatggcatggtgctgtccttatagtaatgagttcttactctactagttcccaggacagctggtcattacaaacagcctggcacctcccctgacctctccttgctttcctccctcaccatgtgatctctgtaaatgccagctccccttccccttctccatgagtggaaacagcctcaggccttcacccgaagtggatgctggtaccctacttcttgtacagcctgcgtaactgtgagtcaaataaacctcttttctttataaattacgcagcctcaggtattccttaatagcaacacaaatggactaatat

SEQ ID NO: 20 - DNA, 396 bp, Homo sapiens
  source 1..396: mol_type = genomic DNA
tagtggacactttttaggatgtctgccaagcctactttttacaaactttttttgccctgacataagagtttgccagttacccctgtacccacctttcaccagagctgacccaaacagaaccaatgagattcttgcctggcaatctggaggtattacatagccaattggcagatcttatgctctagaagagatctaaatgcaggagctgtgggttggctgttttctggcctgccagtggatgaagaaacaaaaaggtctggaaggatagcaatgtgcagacacagaaagttctatggcattctagctcctggttccaattccttcctaaagttcagctgctttctgcctacagttcttatgatatacctctacattccctattttagcccaagttgg

SEQ ID NO: 21 - DNA, 359 bp, Homo sapiens
  source 1..359: mol_type = genomic DNA
ttatggactcaatatgtaccctcaaattttgtatgttgaacctaatcctcaattgttggaatttgaaagtggggtctttgggaggtaataagattgagatgaggtcatgagtgagggtcaggatgtgaagtgctcttataagaaaaagagaaaccagagctctcgctctctccaccacatgcggactcagcaagaaggcggccatctgtaagccaggaagagagtccccaccagtgaatcaaaactactggcaccttgatgttggacttcccaatctctagaactgtgagaactaaatttctgttgttcaagccacctagtctatagttgttttttaaaatagcagcttgagctacgac

SEQ ID NO: 22 - DNA, 436 bp, Homo sapiens
  source 1..436: mol_type = genomic DNA
tgctatggtttggatatggtttgtttatccccaccaaaattcacattgaaatttcttccccagtgtagtagtgttgggaggtgggacctagttggggaatggcttggtgccactctctaggtagtggctgagttcttgctgtggcgagaatgaattagttcttgcgggaatgaattcttaatagttcctgccagagtgagtttttagaaagccaggatgccccttgggttttgtctcttttcacatgtccactttccctttgaccttctctgctgtgttttgacctagcatgagaccttcaccagaagccaagtagatgtcagcaccgtgcttctctaactttccacctgcaaaactgtgagctaaataaacctcttttctttataaattacctagcctctgtattctgttatagcaacacaaaatggactaagac

SEQ ID NO: 23 - DNA, 373 bp, Homo sapiens
  source 1..373: mol_type = genomic DNA
ccagcctcgcacctaagaacgccgtgggatgcagtttcagacagtgagaaatctgctggggatgcctgggaaagggacatacctggcaggtgccgctccatggccttcttgctgccttgaattgggacatgagcctgagtcgcagcagccgtcttgtgactgagaggtggcaagcaggaggaagaggctaagagaatagagacactgttgagctgctgaagcacagcaagcggccacttctctatgcttcttgctatttgactgttaagctagtgcaagttacttttctggtcagaatgaattcataactgaaacatgctttaaagtatgactattattattaaatgcagcataagctttccatagctgctac

SEQ ID NO: 24 - DNA, 598 bp, Homo sapiens
  source 1..598: mol_type = genomic DNA
tgtaaggtatatagatgtgatttggtcaaggtagagaccgaggcggatatacaggcctgcacggctcagtgagtttggtgcgcaggcgcacacctccgcttgttacataacctgtttgtgtaagttcatacttggctctgagccactattgtctgtaaaaggtataactgccctgctgacgccgtacaggtgcttttgaggctcagcttggctcgacatggcttgatgtggtgggtgcgctggcgcccagagaaagggagacagccaaagctgtccatcttgtagacagacaggagggagccaggacacagctctgcttgcttgtgccagagaaagaaagagttaagctgctgaccctgaaggcaagggagagccgggggtcacaggagccacagagccaaagcaaatagcagagataaaggtggacagggtgagagagctaatgtgagtaagctgctaataaaaagccattgatgagaactgctgctgaataaaaccatattcacctgcctaggccccctgcaccaaagcgtttctgctcatccaccccactcaccttggacttcagcatgggctggacctggaccggggtctgaca

SEQ ID NO: 25 - DNA, 489 bp, Homo sapiens
  source 1..489: mol_type = genomic DNA
tattgcttttcagcaacaaatccatctttctttccccctctctgttatactggggcccaattctacagacattacttctttgccagttggcttaatgttaggtcctgtcaatagagggcattggagacacattgcaagtctggaagaggaaaaaaggactttttcttcttccagtgttgtatttttctacttggtggcaagtagatcaataagcaagggtcctggcagtgttaaacagaggaggacagttatttgatttgggagcaagtgctccccaatatcagtcaacttgccctgtccattgacaagagtccgaatcttaaccttgcaggggctcccctcttccaagtttctgcgttccttcatcatttacttttctttgaactttcagagtagttactgatctctgcatttactactttaagagttttttatattaaaacttccctattccaattgccaatgtggtttatgtcttctgactgaacc

SEQ ID NO: 26 - DNA, 691 bp, Homo sapiens
  source 1..691: mol_type = genomic DNA
gtagcagagactggctagatatttttcaaacaattttcatgatcctgggtgaagccatgtgtctgaggatgaaaggggatgcagattgatgtgacatgtggcacctccaggcctggtccttaaaaacctctcacccaatttttttttctttgagacagggtctcaatctgttgcccaagctggagtgcaattgtgcaatcatagctcaccgcatcctcaaactcctggttcaaggaatcctcctgtcttagcatcctgagtagctgggactacaggtgcacaccagaatgcctggccaattttttttttttcattttttgcagtgacaaggtctccctatgtgttgctcatgctggtcttgaactcccggcctcaggcaatcctcctgtctctgcttcccaaagtgctggaattacaggcgtaagccactgtacctggcctctcccatccaatctttatccctctccttcctttttcttctcctggcttttgacccagctaccaaggatccactgggggaactccaaggaactggtggataatggagccccaagctggaaggcgcttggtttccaaataactgcagcctctacaccaagccacattggactgtgacgtgaaccagaaacaaacttttattatgttagatttggaggttgtttgttttagcagttaggttacactgactcac

SEQ ID NO: 27 - DNA, 472 bp, Homo sapiens
  source 1..472: mol_type = genomic DNA
tgtactgggtcgaatggcgtccccccccaccaaattcatgtctgcatggaacctacaaatgtgactttgtttggaaagagggtctttgcagatgtaattaagatgtactccacaggcctggggagatcaaggctaacagcatgttgtatgaggatggccccagtccaatgactgatgcccttatatgagggaaatctggacacagacacacagagaaggccacgcggtggtggaggcagagatgggagagatgtgtctacaagccaaggagcgagcaggaccgccggccaccaccaggagctggcaggggcaaggaagtcattcttccctgggggcttcagagggagcacggtcccagtacaccttgagtgttggccttctggcctccagagctgtgagagaatccattttggttgtttgaagctgctgagtttgtggcgttttgttacggctgtcccaggaaaggaataca

SEQ ID NO: 28 - DNA, 5058 bp, Homo sapiens
  source 1..5058: mol_type = genomic DNA
tgatgccatgacttggacaaaatgcccattgcctctgggtcctgctttcttcacccagtgctgccttattggactccttgtgcctctccttggctggggaaatcagaatacacagtggtaagtcatgatttctaatccagtgctccagacgtctgtctgttgtagcacagtgagaagttgacttatatttctctcatttttgtcattttcttctgtttttccatctttcccttctgccttttttgagtgtggtgaaagcccctcattctctcctgagaatcatacctgagttgaaatcccaaagcctctagcaccagcaggtgtcccttccattatcatgagaaaccgtcaatttggctgttgttctccagaaccctcaactctggggtatgctctgtgttactatggtaataattccacccactttcttccaaaaaaaaaaatggcaatattttacaacttaaatttcaatggcctctttggagaacgtagaatttttccaaattgcctcattctgcgaagggcaattgaatcctgtgccaccaagatctcatccaagcaatagatgctttgttagtggtggtgggcatttggtacaaagaggcagaaaaaaaaaatgctctctctcaaaaacttttcagtgaccattgcttctttcaaagacagtcatccttccccaatctgtgacctgtgtcttctcatcacctatcccccaatctctcttttctaccatcttccatgttcccttcagctccctttaatccttggatcttcctctttcaaccctctatctcctccacctttccatccactcctgccagacctttcctctcccactctggcccctcaattccttataatcccttgagctttaagtcctgtcccttcatgggaggctctagagaagcccaggggcactaagagcaacagcctgaggctaaaaaggctcaccggaaacaggctgaatgttttatcccctcaggtgggctctggagacaccagatgaccgcttggtctctcctccatgcctcccttgaaattgagtccacagcttccctagaattatacaccagagccaagggtagcttatgggtctcctacacaaatattggtgaagatgcttcagttctcatactgaacaggttacttcaacttgccccatctaccaaaaatattatccttgtaaaaatataaacgtgaggcaatgatgatactttgtttgtttgtttatttgtttgttttttgagatggaatctcactctgttgccaggctggtgtacagtggtgcaatctcgactcactgcaatctctacctcccaggttcaagcgattctcctgccgcagcctccaaagtagctgggattacaagcacatgccaccatgctcagctaatttttgtatttttagtagacactgtgtttcaccatgttggccaggatggtctcgatcttctgacttaatgatctgaccgccttggcctcccaaagtgctgagattacaggcatgagccaccgtgcccagtcaatactctgtttttttatatgaatcagtgagttttgcatttctgtgattatgcctaagtcatggctaaaatttaaaaattaaagctgtaagttggctatgtctgcctatatgtttatgtatgtgtgtttgtgcatgtgcacatacattttgtttgtgtaatattttcctacctccagatggtattaccacattaaattataaaacgccttaaataagctccttctggattgtcttagagatgaatgagagcctacataagttaagtattcttgaacttccttgaaattaggaaaatcaaatttctaatatcatcaatgtgaaaaaagtatttttataaaagcttaacaaagtgtttaaaaattcaagttcacctaatttagataaacctttggccattgagagtagtttaatataattgatttaataaatatagctatgtcttctgagttgtccacatggtaagaatacatttttgttctacttgagtatttttttcctaaacctatacaaatatactgaatctctttgcttgataatctaaatatattcttggtatattctaactatatgtttgatatattcatgacacattatgtctcatgattatcatgttatttctttttttttttttttttttttttttttttgagatggagtctcactctgttgcccaggctggagtgcagtggcacgatctcggctcactacaacctccgcctcccaggttcaagagattctcctgcctcagcctcccgagtagctgggactacaggtgactgccatcacacctggctaatttttgtatttttagtagagacggggttttaccatgttggccaggctggtctccaactcctgaccttatgcttgtgatccgcctgcctcagcctcccaaactgctgggattacaggcatgagccactgcgcccagataatcatgttatatctgaaaatgttttctctataaaaactatgcccatccactttcataaatttgatgtaatatccacccttttttgaaaataaaattaattattatgtttacacatattttctctgaaactttttgtagcaactttaagtgccataacgacaattaaatttttctgtcaatgtattatttttatagcaaactttcatcagatcttttactttcaaaaattatcaaaaatatgttaaccacagcaattttaagtcttttgttatctatggaggactttgtttccacaaaaaaggactgactcagaagtatgtggaaaaggattatgctaggtgctgttgtgttaaagtttctaattacgttgtttaagtaactttgggaccaaaacaatgaactgagttaggaattccagaactctagttgaaaaagagatgggttcataagcctactaatccgggatcaagtgggacaagaactaattccacaggagtgaatgaactgatgatagataattatgggacttttttggaatagggctatagggctattcctttaatgtactattttctttttttttttgagacagagttttgctcttatgcccaggctggagtgcaatggcgtgatctcagctcactgcaacctctgcctcccaggttcaaacaattctcctgtctcagcctcccaagtagctgggattacagggtagtgccaccacacttggctgtttttgtatttttagtagtgacgggttttcaccatgttggccaggatggtctcgatctcttgacctcatgatccacccgcctcggcctcccaaagtgctggaataacagatgtgagccaccacgctcggcctaatatcctattttctggacataagaaaacccctcctcttttttttaagctattcatcactcataacaatttcatagaccatgctaatttcttgtaaacagaaatgaaacttctttcttttcttccctgactcacccattcaaaatttggaaattctccttgagtcttcttattttcatggcaatatggttatttgcatagattcagtaagagtctgtcctctttgttaacagggcacaaaaacattggtaatacaaccaaggctttgcctggatgtcatatttgagagtgacgcttatttaatcagatatgaccagacacttttaaggaactaaggttgaccttatagggccaatgcttacaaagctcctcttgcgaaaactggcttgaaacctggctttgcagggtttgcagctttgcaggtgagcaaggaatgtcacttcccagcaggctctggaatcttaggatatttgtgggatctcaagaagaaaggaatgcctgccaaatcgataggtactgcatgtgaaatctgatgtgagttgttggcttggcttcttatcctcaagaggctttgaaaagtccaatttgacattctttatgaaaacttccagcaaagcaaatataatatgatctatatgtaaattgctgctcttgcagcacctatgcaaataaccaggccgaatctcatgagaccaaccttaatttgtgatcaagaataatctttctttgagattgtctttgatcaaaagaggaggagtaactgtagaaagaaaatttgtgtttccatgggaaatcatagcacatctttctggattatcagattctgatcctgttaatttgtgtttgagctatttagtgcctcttggtaaactgagagtaactgatagttatgcaaactctctcttctctagtagagatttaattgactttctctcacactatgggacaatcagtttttgtaactactaataaattgtacttgaatctgttatcttgcacaaattgtccacttttactaaattttcaaatcttccaatttccctcaatatctagccataaacccctcaactaacgttccaatttttctccttttttgttactcatagtcactgagaaaactttactatccagatgacagttaagaccctaggttgccttgtggctggcccccagtctctgaagtattactacaatctcaagtctgatgacttgttataaacatataaagacccatcattgctagcagactatgcatggttctctttttctctggacaagccctgactggaccacaaaaaaaggatgggaaatgctcaggtcacgcatgttctgatagaagaggtaatccaggctctgggcaacattaccaagagcatcaaaacgctcaaagagcaatgctacatgatcaaaagggctcatccactaaccccctgaaatcccctgaaatggatacccttgggttcaacttctgtctttctcagtttaatctctctttctatattaacatttctcttttttccttttaggtatcccacttctaagatgcctgatctgaaggacagtaaaacaactgacctttgccagcatgtaaaacacatggtttaactagtcctccaggaacaacactgagcaatcctgacctgggactactttactcggccatctcctacttgagatgctccttgtctctctgttcaaggacaccttttctgagcctttcttgaacaagagtggag

SEQ ID NO: 29 - DNA, 538 bp, Homo sapiens
  source 1..538: mol_type = genomic DNA
tgtcagaggtgtgtgaaccagagcaactccgtctagaacaggagctgggtaaaatgaggctgcgacccatggggctgcattcccagacagttaaggcattctaagtataggaggttggcagaagatacaggtcataaagaccttgctgataaaacagtttacttcaaagaagccaactaaaaccaaccaaaaccaagatggtgatgagtgacctctggttgtcctcactgctacactcccaccagtgccatgacagtttacagatgacatggcaacatcaggaagttaccctatatggtctaaaaaggggaggcataaataattcaccccttgtttagcatataatcaagaaatggccataaaaataagcaaccagcagccttccgggctgctgtctatagggtagccattttttgttcctttacttacctaatgaacttgctttcactttactctatggactcgccctgaattccttcttacacgagatccaagagccctttcttgaggtctgggtctgcacccctttcctgcaaca

SEQ ID NO: 30 - DNA, 416 bp, Homo sapiens
  source 1..416: mol_type = genomic DNA
gttaaagcaaacttaaaatgaacacctgggcaaacaaaagcaaacaggccttcagaaaggctgtaaccccccttaaactgccaactatggggaacttaactggagtcgtttcagatgggtgcttacgttaggcacaaacaaaacttaacttcggccagtcatgagcagccagctgacagacgggtacacgactaggaattttccaacaaggtaaaccaaaaaacataatttgacaactgcaacaaatcaaataatgtccttattccacttccatattcaccctataaatacctgcctctgacacttttccatcataacatgaaatgtctttcattttgatgtttcccacttcatgacttgcttcttcctcaaataaagtcttcaaaattcgattggacctcagatttttctttgac

SEQ ID NO: 31 - DNA, 1613 bp, Homo sapiens
  source 1..1613: mol_type = genomic DNA
tgatggcagcggcggcccaccttgagtggccgctgcatgacactggctacaatgggggaggcacagccagggctgagtgctccatagaactggtgggagccgggaacaggtggaagccatgcccccttccaagttggaggagctggagccctgccctcccaggggcagctgtggcagagcagccagctgtggactcaggcatcactgcactctcagggccccaggaaacacccttgccccttcaggctcaaaagtgcctgctctcgctgcctgacctctgcccattcccagtgcccactccaatttcagagcaaagttgtggccaagcctgggggctgtcacgactcggctgggtgtgtgcacgcttggggcagcactgaaacaccagccccctatcacctcagcctcctccagattttgggtgctcacaaacatgggagggagggtgaggtgggggctaagggtggctcagcgcagtcttgcaggcaccacttggcacaaagagccagggcaccataggcaccatggagagcaggttaatggtggtaggatgcaaacaggctcctgggcagaaaggagcagatccccagtaaagccccatcttcaggccagggacggcctgaagcctgccggagggctgccatggagtgagaacttaatggagcttgctctggggcctcccatgactgctcacgggccgatcagcacctactttctcccctcttaagcccataaaaatcttggactcagccagacttgagcagatgatgggatggcctgcctacagaaaggaactacccattgtgggtctcctgtctcctgagagctgagtggacaacgggacaatcagcctgcagaaaggagctacccactgtgggtctcctttctgctgacagctgaacagatattgggatgacctgcctatggagaggagctaaccactgtgggtctcctctgagctgttctgttgctcaataaagtacctcttcaccttgctcaccctctacttgtctgcataactcattcttcctggatgcagtacaagaacttgggacctgccgaatggtggggctgaaagagatgtaacacaaacagggctgaaatacaccccctgcttgccacatggcaggcaacaagaaaaagataagaaagaaggagagaagagtttcagcccttcagacaacccagacctgggagcaccctgagccagggctgtgatgccctttttggggttctgtggttcctggcatctccaagtttctgggtgccaccacattccctggtgccagccatggaagctacttgcagtgtgcctcatccagccacagccttgcagggagctggtgcctggcactgcccaccctgctgcagccagagtgcctggctgtgggcagtggctggaccccatgcttgctcattcacacaatcctcaccgctctgtgcctggctcacccttggcaggcatgggatccaggtttgtcacgcaagctgagttcaggctgccaggccgagtgggcacatcaagcacagtgggccaaagcaaaattcagacaaaggcaccaccgaccacagaggtttctggacagaaaagcgacacctgaaggatcctatga

SEQ ID NO: 32 - DNA, 409 bp, Homo sapiens
  source 1..409: mol_type = genomic DNA
gcaatcaggagcctgggtaaagcagactgtgtgctctgatgggggtgggcctcacccgatcagttgaaggcaacagggcttggaacgcacactccaagatacagtgccttggcatagtaaatattttcagctgaaggaatttaagaaatggcaggtccaagaagaactcactgacctgccccttctctgaagtagatcataagcccctcacgtaagaggtgccctccctacacccagcggaaaggaacagcctcatctccagagacagaggatctgagaggggccccagtacacaggtcttgccgagacccccacgggctcactgttcagctcatcttttgttgctctgtcacagtttcccacgactttccactcttcatcaaacccagcataaaaacgctcaggccta

SEQ ID NO: 33 - DNA, 625 bp, Homo sapiens
  source 1..625: mol_type = genomic DNA
tgttatgggccaaattgtgccacccctcccaaatgcatatgtttaaattctaattcccaggacctcctaatatggctgtatttggagacaggatctttaaagtggtcgttgtaggcccagcacagtggctcatgcctgtaatcccagcactttgggggaccaaggtgggcagattgcttgagcctaggagttcaaaaccagtctgggcaacatggtgaaaccccgttgctacaaaaaatacaagaattaaccaggcacggtggtgcacacctgtaatcccagctactcaggaggctgaggcgggaggatgcttaagcctgggaggtggaagttgcagtgagctgagatcacaccactgcactccagcctgagtgacagagcgagaccctgtctcaaaaacaaaagaagaggaggaggagattaggacccagacacacagaaggagaaccaggtgaagacacagggagaagaggatcatctacaagccaagacaggaggcctcagaagaaattaaccctgccaacaccttgatcttggacatccagcctccggaactgtgagaaaatgcattctgttgctgaagccacccagtcctttgttatggcagccccagcaaactaacaca

SEQ ID NO: 34 - DNA, 345 bp, Homo sapiens
  source 1..345: mol_type = genomic DNA
gggatggttttttcatgtgtcaactttgctaggcaatagtcccctgttactcaatccaacactaatctagatatttattgaaggtattttgtagatttcgttaaagtccgtaaccagttgattttaagtaagggaaagtatcctagataatctaggtgaaattgattcaatatcagttgaaaggtcttaagagtggaattaaggcttccctgataaacatgaatttctacctatagacagcagcttcagcagctttggcttctgctagagagtcccagcctgcccttcctggcagcctgccttatggattttggaattgcctagccatgagccaattccttgcag

SEQ ID NO: 35 - DNA, 23 bp, synthetic construct
  misc_feature 1..23: note = Amorce d'amplification
  source 1..23: mol_type = other DNA
tgtacaaaactcaaatggtcttc

SEQ ID NO: 36 - DNA, 23 bp, synthetic construct
  misc_feature 1..23: note = Amorce d'amplification
  source 1..23: mol_type = other DNA
atgaccaacttagatttccttga

SEQ ID NO: 37 - DNA, 22 bp, synthetic construct
  misc_feature 1..22: note = Amorce d'amplification
  source 1..22: mol_type = other DNA
gccagagaggcataatgaagca

SEQ ID NO: 38 - DNA, 23 bp, synthetic construct
  misc_feature 1..23: note = Amorce d'amplification
  source 1..23: mol_type = other DNA
gattctaagcctccccctcattt

SEQ ID NO: 39 - DNA, 22 bp, synthetic construct
  misc_feature 1..22: note = Amorce d'amplification
  source 1..22: mol_type = other DNA
tggctcatagggattccagact

SEQ ID NO: 40 - DNA, 23 bp, synthetic construct
  misc_feature 1..23: note = Amorce d'amplification
  source 1..23: mol_type = other DNA
agcaagttgtcaagagccaatct

SEQ ID NO: 41 - DNA, 20 bp, synthetic construct
  misc_feature 1..20: note = Amorce d'amplification
  source 1..20: mol_type = other DNA
cactctaggaatcttaggca

SEQ ID NO: 42 - DNA, 19 bp, synthetic construct
  misc_feature 1..19: note = Amorce d'amplification
  source 1..19: mol_type = other DNA
tgaaaccaatagtccagtg

SEQ ID NO: 43 - DNA, 24 bp, synthetic construct
  misc_feature 1..24: note = Amorce d'amplification
  source 1..24: mol_type = other DNA
ttctactgttcactgctatcctcc

SEQ ID NO: 44 - DNA, 23 bp, synthetic construct
  misc_feature 1..23: note = Amorce d'amplification
  source 1..23: mol_type = other DNA
cctgtggcagctttttgaagtaa

SEQ ID NO: 45 - DNA, 22 bp, synthetic construct
  misc_feature 1..22: note = Amorce d'amplification
  source 1..22: mol_type = other DNA
agagcagaagaagatggatact

SEQ ID NO: 46 - DNA, 21 bp, synthetic construct
  misc_feature 1..21: note = Amorce d'amplification
  source 1..21: mol_type = other DNA
catgagctgacatcatccaat

SEQ ID NO: 47 - DNA, 20 bp, synthetic construct
  misc_feature 1..20: note = Amorce d'amplification
  source 1..20: mol_type = other DNA
tctgtactggttgccccaac

SEQ ID NO: 48 - DNA, 23 bp, synthetic construct
  misc_feature 1..23: note = Amorce d'amplification
  source 1..23: mol_type = other DNA
cgtgccaggcctctaatactttt

SEQ ID NO: 49 - DNA, 21 bp, synthetic construct
  misc_feature 1..21: note = Amorce d'amplification
  source 1..21: mol_type = other DNA
agggaagaccccaagatgatg

SEQ ID NO: 50 - DNA, 21 bp, synthetic construct
  misc_feature 1..21: note = Amorce d'amplification
  source 1..21: mol_type = other DNA
catgcaaagtccaacgagagg

SEQ ID NO: 51 - DNA, 19 bp, synthetic construct
  misc_feature 1..19: note = Amorce d'amplification
  source 1..19: mol_type = other DNA
gggtggctgcatcctatgg

SEQ ID NO: 52 - DNA, 24 bp, synthetic construct
  misc_feature 1..24: note = Amorce d'amplification
  source 1..24: mol_type = other DNA
ctggtcaggaaaaaatttgccttc

SEQ ID NO: 53 - DNA, 24 bp, synthetic construct
  misc_feature 1..24: note = Amorce d'amplification
  source 1..24: mol_type = other DNA
acatgacattgtctgaactttggg

SEQ ID NO: 54 - DNA, 24 bp, synthetic construct
  misc_feature 1..24: note = Amorce d'amplification
  source 1..24: mol_type = other DNA
taggaccatgcagatactagtgac

SEQ ID NO: 55 - DNA, 18 bp, synthetic construct
  misc_feature 1..18: note = Amorce d'amplification
  source 1..18: mol_type = other DNA
gaactccacaaaccttga

SEQ ID NO: 56 - DNA, 20 bp, synthetic construct
  misc_feature 1..20: note = Amorce d'amplification
  source 1..20: mol_type = other DNA
gctagaagctttggatatct

SEQ ID NO: 57 - DNA, 20 bp, synthetic construct
  misc_feature 1..20: note = Amorce d'amplification
  source 1..20: mol_type = other DNA
tggctgttacaactttcatg

SEQ ID NO: 58 - DNA, 19 bp, synthetic construct
  misc_feature 1..19: note = Amorce d'amplification
  source 1..19: mol_type = other DNA
tctccctattctgagcaca

SEQ ID NO: 59 - DNA, 25 bp, synthetic construct
  misc_feature 1..25: note = Sonde
  source 1..25: mol_type = other DNA
atgaccaacttagatttccttgagt

SEQ ID NO: 60 - DNA, 25 bp, synthetic construct
  misc_feature 1..25: note = Sonde
  source 1..25: mol_type = other DNA
gtcaagggtaaagctgtgaaagttt

SEQ ID NO: 61 - DNA, 25 bp, synthetic construct
  misc_feature 1..25: note = Sonde
  source 1..25: mol_type = other DNA
ggaagaccatttgagttttgtacac

SEQ ID NO: 62 - DNA, 25 bp, synthetic construct
  misc_feature 1..25: note = Sonde
  source 1..25: mol_type = other DNA
actcaaggaaatctaagttggtcat

SEQ ID NO: 63 - DNA, 25 bp, synthetic construct
  misc_feature 1..25: note = Sonde
  source 1..25: mol_type = other DNA
aaactttcacagctttacccttgac

SEQ ID NO: 64 - DNA, 25 bp, synthetic construct
  misc_feature 1..25: note = Sonde
  source 1..25: mol_type = other DNA
gtgtacaaaactcaaatggtcttcc

SEQ ID NO: 65 - DNA, 25 bp, synthetic construct
  misc_feature 1..25: note = Sonde
  source 1..25: mol_type = other DNA
gaagatatgggccagaacttgtata

SEQ ID NO: 66 - DNA, 25 bp, synthetic construct
  misc_feature 1..25: note = Sonde
  source 1..25: mol_type = other DNA
caggacctgagttaagccaagaata

SEQ ID NO: 67 - DNA, 25 bp, synthetic construct
  misc_feature 1..25: note = Sonde
  source 1..25: mol_type = other DNA
acctgagttaagccaagaatacagt

SEQ ID NO: 68 - DNA, 25 bp, synthetic construct
  misc_feature 1..25: note = Sonde
  source 1..25: mol_type = other DNA
tatacaagttctggcccatatcttc

SEQ ID NO: 69 - DNA, 25 bp, synthetic construct
  misc_feature 1..25: note = Sonde
  source 1..25: mol_type = other DNA
tattcttggcttaactcaggtcctg

SEQ ID NO: 70 - DNA, 25 bp, synthetic construct
  misc_feature 1..25: note = Sonde
  source 1..25: mol_type = other DNA
actgtattcttggcttaactcaggt

SEQ ID NO: 71 - DNA, 25 bp, synthetic construct
  misc_feature 1..25: note = Sonde
  source 1..25: mol_type = other DNA
gtaagattctaagcctccccctcat

SEQ ID NO: 72 - DNA, 25 bp, synthetic construct
  misc_feature 1..25: note = Sonde
  source 1..25: mol_type = other DNA
gattctaagcctccccctcatttaa

SEQ ID NO: 73 - DNA, 25 bp, synthetic construct
  misc_feature 1..25: note = Sonde
  source 1..25: mol_type = other DNA
ctaagcctccccctcatttaaagga

SEQ ID NO: 74 - DNA, 25 bp, synthetic construct
  misc_feature 1..25: note = Sonde
  source 1..25: mol_type = other DNA
atgagggggaggcttagaatcttac

SEQ ID NO: 75 - DNA, 25 bp, synthetic construct
  misc_feature 1..25: note = Sonde
  source 1..25: mol_type = other DNA
ttaaatgagggggaggcttagaatc

SEQ ID NO: 76 - DNA, 25 bp, synthetic construct
  misc_feature 1..25: note = Sonde
  source 1..25: mol_type = other DNA
tcctttaaatgagggggaggcttag

SEQ ID NO: 77 - DNA, 25 bp, synthetic construct
  misc_feature 1..25: note = Sonde
  source 1..25: mol_type = other DNA
atggctcatagggattccagactcc

SEQ ID NO: 78 - DNA, 25 bp, synthetic construct
  misc_feature 1..25: note = Sonde
  source 1..25: mol_type = other DNA
ggctcatagggattccagactccca

SEQ ID NO: 79 - DNA, 25 bp, synthetic construct
  misc_feature 1..25: note = Sonde
  source 1..25: mol_type = other DNA
ctcatagggattccagactcccatt

SEQ ID NO: 80 - DNA, 25 bp, synthetic construct
  misc_feature 1..25: note = Sonde
  source 1..25: mol_type = other DNA
ggagtctggaatccctatgagccat

SEQ ID NO: 81 - DNA, 25 bp, synthetic construct
  misc_feature 1..25: note = Sonde
  source 1..25: mol_type = other DNA
tgggagtctggaatccctatgagcc

SEQ ID NO: 82 - DNA, 25 bp, synthetic construct
  misc_feature 1..25: note = Sonde
  source 1..25: mol_type = other DNA
aatgggagtctggaatccctatgag

SEQ ID NO: 83 - DNA, 25 bp, synthetic construct
  misc_feature 1..25: note = Sonde
  source 1..25: mol_type = other DNA
tgaaaccaatagtccagtggtggcc

SEQ ID NO: 84 - DNA, 25 bp, synthetic construct
  misc_feature 1..25: note = Sonde
  source 1..25: mol_type = other DNA
ttccagtgatttagataaaatccct

SEQ ID NO: 85 - DNA, 25 bp, synthetic construct
  misc_feature 1..25: note = Sonde
  source 1..25: mol_type = other DNA
tttctgcctaagattcctagagtgc

SEQ ID NO: 86 - DNA, 25 bp, synthetic construct
  misc_feature 1..25: note = Sonde
  source 1..25: mol_type = other DNA
ggccaccactggactattggtttca

SEQ ID NO: 87 - DNA, 25 bp, synthetic construct
  misc_feature 1..25: note = Sonde
  source 1..25: mol_type = other DNA
agggattttatctaaatcactggaa

SEQ ID NO: 88 - DNA, 25 bp, synthetic construct
  misc_feature 1..25: note = Sonde
  source 1..25: mol_type = other DNA
gcactctaggaatcttaggcagaaa

SEQ ID NO: 89 - DNA, 25 bp, synthetic construct
  misc_feature 1..25: note = Sonde
  source 1..25: mol_type = other DNA
ctgtggcagctttttgaagtaagga

SEQ ID NO: 90 - DNA, 25 bp, synthetic construct
  misc_feature 1..25: note = Sonde
  source 1..25: mol_type = other DNA
atggttagtgcagagtaaagtttgg

SEQ ID NO: 91 - DNA, 25 bp, synthetic construct
  misc_feature 1..25: note = Sonde
  source 1..25: mol_type = other DNA
aggatagcagtgaacagtagaatgg

SEQ ID NO: 92 - DNA, 25 bp, synthetic construct
  misc_feature 1..25: note = Sonde
  source 1..25: mol_type = other DNA
tccttacttcaaaaagctgccacag

SEQ ID NO: 93 - DNA, 25 bp, synthetic construct
  misc_feature 1..25: note = Sonde
  source 1..25: mol_type = other DNA
ccaaactttactctgcactaaccat

SEQ ID NO: 94 - DNA, 25 bp, synthetic construct
  misc_feature 1..25: note = Sonde
  source 1..25: mol_type = other DNA
ccattctactgttcactgctatcct

SEQ ID NO: 95 - DNA, 25 bp, synthetic construct
  misc_feature 1..25: note = Sonde
  source 1..25: mol_type = other DNA
agatccaacatgagctgacatcatc

SEQ ID NO: 96 - DNA, 25 bp, synthetic construct
  misc_feature 1..25: note = Sonde
  source 1..25: mol_type = other DNA
gatgatgtcagctcatgttggatct

SEQ ID NO: 97 - DNA, 25 bp, synthetic construct
  misc_feature 1..25: note = Sonde
  source 1..25: mol_type = other DNA
gaggttggggcaaccagtacagatt

SEQ ID NO: 98 - DNA, 25 bp, synthetic construct
  misc_feature 1..25: note = Sonde
  source 1..25: mol_type = other DNA
aatctgtactggttgccccaacctc

SEQ ID NO: 99 - DNA, 25 bp, synthetic construct
  misc_feature 1..25: note = Sonde
  source 1..25: mol_type = other DNA
ccccaagatgatggactctggtgat

SEQ ID NO: 100 - DNA, 25 bp, synthetic construct
  misc_feature 1..25: note = Sonde
  source 1..25: mol_type = other DNA
cactgccatcactttgggaaagact

SEQ ID NO: 101 - DNA, 25 bp, synthetic construct
  misc_feature 1..25: note = Sonde
  source 1..25: mol_type = other DNA
aagcagcctctcgttggactttgca

SEQ ID NO: 102 - DNA, 25 bp, synthetic construct
  misc_feature 1..25: note = Sonde
  source 1..25: mol_type = other DNA
atcaccagagtccatcatcttgggg

SEQ ID NO: 103 - DNA, 25 bp, synthetic construct
  misc_feature 1..25: note = Sonde
  source 1..25: mol_type = other DNA
agtctttcccaaagtgatggcagtg

SEQ ID NO: 104 - DNA, 25 bp, synthetic construct
  misc_feature 1..25: note = Sonde
  source 1..25: mol_type = other DNA
tgcaaagtccaacgagaggctgctt

SEQ ID NO: 105 - DNA, 25 bp, synthetic construct
  misc_feature 1..25: note = Sonde
  source 1..25: mol_type = other DNA
gagggcagtttggaacagttggaac

SEQ ID NO: 106 - DNA, 25 bp, synthetic construct
  misc_feature 1..25: note = Sonde
  source 1..25: mol_type = other DNA
tgagagacgattatctggaagaaga

SEQ ID NO: 107 - DNA, 25 bp, synthetic construct
  misc_feature 1..25: note = Sonde
  source 1..25: mol_type = other DNA
tcacagcttgagaatgtggtaggag

SEQ ID NO: 108 - DNA, 25 bp, synthetic construct
  misc_feature 1..25: note = Sonde
  source 1..25: mol_type = other DNA
ggaatggggggcatggaattaaagc

SEQ ID NO: 109 - DNA, 25 bp, synthetic construct
  misc_feature 1..25: note = Sonde
  source 1..25: mol_type = other DNA
gttccaactgttccaaactgccctc

SEQ ID NO: 110 - DNA, 25 bp, synthetic construct
  misc_feature 1..25: note = Sonde
  source 1..25: mol_type = other DNA
tcttcttccagataatcgtctctca

SEQ ID NO: 111 - DNA, 25 bp, synthetic construct
  misc_feature 1..25: note = Sonde
  source 1..25: mol_type = other DNA
ctcctaccacattctcaagctgtga

SEQ ID NO: 112 - DNA, 25 bp, synthetic construct
  misc_feature 1..25: note = Sonde
  source 1..25: mol_type = other DNA
gctttaattccatgccccccattcc

SEQ ID NO: 113 - DNA, 25 bp, synthetic construct
  misc_feature 1..25: note = Sonde
  source 1..25: mol_type = other DNA
agtccctaactgtctgcaaacccac

SEQ ID NO: 114 - DNA, 25 bp, synthetic construct
  misc_feature 1..25: note = Sonde
  source 1..25: mol_type = other DNA
actgtctgcaaacccacaatggacc

SEQ ID NO: 115 - DNA, 25 bp, synthetic construct
  misc_feature 1..25: note = Sonde
  source 1..25: mol_type = other DNA
caatggacctgttgcatgtgtaaga

SEQ ID NO: 116 - DNA, 25 bp, synthetic construct
  misc_feature 1..25: note = Sonde
  source 1..25: mol_type = other DNA
gtgggtttgcagacagttagggact

SEQ ID NO: 117 - DNA, 25 bp, synthetic construct
  misc_feature 1..25: note = Sonde
  source 1..25: mol_type = other DNA
ggtccattgtgggtttgcagacagt

SEQ ID NO: 118 - DNA, 25 bp, synthetic construct
  misc_feature 1..25: note = Sonde
  source 1..25: mol_type = other DNA
tcttacacatgcaacaggtccattg

SEQ ID NO: 119 - DNA, 25 bp, synthetic construct
  misc_feature 1..25: note = Sonde
  source 1..25: mol_type = other DNA
ctgcatcctatggtgtttctacatg

SEQ ID NO: 120 - DNA, 25 bp, synthetic construct
  misc_feature 1..25: note = Sonde
  source 1..25: mol_type = other DNA
ataatcttttccggcatgttggtat

SEQ ID NO: 121 - DNA, 25 bp, synthetic construct
  misc_feature 1..25: note = Sonde
  source 1..25: mol_type = other DNA
taaagatagtgtttcctattgtgtc

SEQ ID NO: 122 - DNA, 25 bp, synthetic construct
  misc_feature 1..25: note = Sonde
  source 1..25: mol_type = other DNA
catgtagaaacaccataggatgcag

SEQ ID NO: 123 - DNA, 25 bp, synthetic construct
  misc_feature 1..25: note = Sonde
  source 1..25: mol_type = other DNA
ataccaacatgccggaaaagattat

SEQ ID NO: 124 - DNA, 25 bp, synthetic construct
  misc_feature 1..25: note = Sonde
  source 1..25: mol_type = other DNA
gacacaataggaaacactatcttta

SEQ ID NO: 125 - DNA, 25 bp, synthetic construct
  misc_feature 1..25: note = Sonde
  source 1..25: mol_type = other DNA
acagagactgcaagagtaatgacat

SEQ ID NO: 126 - DNA, 25 bp, synthetic construct
  misc_feature 1..25: note = Sonde
  source 1..25: mol_type = other DNA
tctgaactttgggaaacaattatgt

SEQ ID NO: 127 - DNA, 25 bp, synthetic construct
  misc_feature 1..25: note = Sonde
  source 1..25: mol_type = other DNA
actttccagttaatcgaatcaatcc

SEQ ID NO: 128 - DNA, 25 bp, synthetic construct
  misc_feature 1..25: note = Sonde
  source 1..25: mol_type = other DNA
ttttaacctagactagttccaactg

SEQ ID NO: 129 - DNA, 25 bp, synthetic construct
  misc_feature 1..25: note = Sonde
  source 1..25: mol_type = other DNA
gtcactagtatctgcatggtcctaa

SEQ ID NO: 130 - DNA, 25 bp, synthetic construct
  misc_feature 1..25: note = Sonde
  source 1..25: mol_type = other DNA
atgtcattactcttgcagtctctgt

SEQ ID NO: 131 - DNA, 25 bp, synthetic construct
  misc_feature 1..25: note = Sonde
  source 1..25: mol_type = other DNA
acataattgtttcccaaagttcaga

SEQ ID NO: 132 - DNA, 25 bp, synthetic construct
  misc_feature 1..25: note = Sonde
  source 1..25: mol_type = other DNA
ggattgattcgattaactggaaagt

SEQ ID NO: 133 - DNA, 25 bp, synthetic construct
  misc_feature 1..25: note = Sonde
  source 1..25: mol_type = other DNA
cagttggaactagtctaggttaaaa

SEQ ID NO: 134 - DNA, 25 bp, synthetic construct
  misc_feature 1..25: note = Sonde
  source 1..25: mol_type = other DNA
ttaggaccatgcagatactagtgac

SEQ ID NO: 135 - DNA, 25 bp, synthetic construct
  misc_feature 1..25: note = Sonde
  source 1..25: mol_type = other DNA
ttattccagtcacctcgagtcattc

SEQ ID NO: 136 - DNA, 25 bp, synthetic construct
  misc_feature 1..25: note = Sonde
  source 1..25: mol_type = other DNA
tcatcctagccgtcgtagagcagag

SEQ ID NO: 137 - DNA, 25 bp, synthetic construct
  misc_feature 1..25: note = Sonde
  source 1..25: mol_type = other DNA
tgcccttctgactccttgacagtgg

SEQ ID NO: 138 - DNA, 25 bp, synthetic construct
  misc_feature 1..25: note = Sonde
  source 1..25: mol_type = other DNA
gaatgactcgaggtgactggaataa

SEQ ID NO: 139 - DNA, 25 bp, synthetic construct
  misc_feature 1..25: note = Sonde
  source 1..25: mol_type = other DNA
ctctgctctacgacggctaggatga

SEQ ID NO: 140 - DNA, 25 bp, synthetic construct
  misc_feature 1..25: note = Sonde
  source 1..25: mol_type = other DNA
ccactgtcaaggagtcagaagggca

SEQ ID NO: 141 - DNA, 25 bp, synthetic construct
  misc_feature 1..25: note = Sonde
  source 1..25: mol_type = other DNA
taagtgggaccaagacacaaaccaa

SEQ ID NO: 142 - DNA, 25 bp, synthetic construct
  misc_feature 1..25: note = Sonde
  source 1..25: mol_type = other DNA
accaagacacaaaccaacatgcctg

SEQ ID NO: 143 - DNA, 25 bp, synthetic construct
  misc_feature 1..25: note = Sonde
  source 1..25: mol_type = other DNA
ttggtttgtgtcttggtcccactta

SEQ ID NO: 144 - DNA, 25 bp, synthetic construct
  misc_feature 1..25: note = Sonde
  source 1..25: mol_type = other DNA
caggcatgttggtttgtgtcttggt

SEQ ID NO: 145 - DNA, 25 bp, synthetic construct
  misc_feature 1..25: note = Sonde
  source 1..25: mol_type = other DNA
ctgaggtccatggcttctttccttg

SEQ ID NO: 146 - DNA, 25 bp, synthetic construct
  misc_feature 1..25: note = Sonde
  source 1..25: mol_type = other DNA
caaggaaagaagccatggacctcag

SEQ ID NO: 147 - DNA, 25 bp, synthetic construct
  misc_feature 1..25: note = Sonde
  source 1..25: mol_type = other DNA
cctttgttttcctactgacaggtcc

SEQ ID NO: 148 - DNA, 25 bp, synthetic construct
  misc_feature 1..25: note = Sonde
  source 1..25: mol_type = other DNA
ttcaaaatatttaactctccaggct

SEQ ID NO: 149 - DNA, 25 bp, synthetic construct
  misc_feature 1..25: note = Sonde
  source 1..25: mol_type = other DNA
gaggtcacatgactctgttgtggac

SEQ ID NO: 150 - DNA, 25 bp, synthetic construct
  misc_feature 1..25: note = Sonde
  source 1..25: mol_type = other DNA
ggacctgtcagtaggaaaacaaagg

SEQ ID NO: 151 - DNA, 25 bp, synthetic construct
  misc_feature 1..25: note = Sonde
  source 1..25: mol_type = other DNA
agcctggagagttaaatattttgaa

SEQ ID NO: 152 - DNA, 25 bp, synthetic construct
  misc_feature 1..25: note = Sonde
  source 1..25: mol_type = other DNA
gtccacaacagagtcatgtgacctc

SEQ ID NO: 153 - DNA, 25 bp, synthetic construct
  misc_feature 1..25: note = Sonde
  source 1..25: mol_type = other DNA
cagctgagatccgttgacgccagcc

SEQ ID NO: 154 - DNA, 25 bp, synthetic construct
  misc_feature 1..25: note = Sonde
  source 1..25: mol_type = other DNA
tccgacatgtgggtgaactcagcca

SEQ ID NO: 155 - DNA, 25 bp, synthetic construct
  misc_feature 1..25: note = Sonde
  source 1..25: mol_type = other DNA
ttctcagccatgtgttttgtgaact

SEQ ID NO: 156 - DNA, 25 bp, synthetic construct
  misc_feature 1..25: note = Sonde
  source 1..25: mol_type = other DNA
ggctggcgtcaacggatctcagctg

SEQ ID NO: 157 - DNA, 25 bp, synthetic construct
  misc_feature 1..25: note = Sonde
  source 1..25: mol_type = other DNA
tggctgagttcacccacatgtcgga

SEQ ID NO: 158 - DNA, 25 bp, synthetic construct
  misc_feature 1..25: note = Sonde
  source 1..25: mol_type = other DNA
agttcacaaaacacatggctgagaa

SEQ ID NO: 159 - DNA, 25 bp, synthetic construct
  misc_feature 1..25: note = Sonde
  source 1..25: mol_type = other DNA
ttgaggcaggacagaaccaggctcc

SEQ ID NO: 160 - DNA, 25 bp, synthetic construct
  misc_feature 1..25: note = Sonde
  source 1..25: mol_type = other DNA
ggacagaaccaggctcctgttagtc

SEQ ID NO: 161 - DNA, 25 bp, synthetic construct
  misc_feature 1..25: note = Sonde
  source 1..25: mol_type = other DNA
agtttactgagcagtgactttgtgt

SEQ ID NO: 162 - DNA, 25 bp, synthetic construct
  misc_feature 1..25: note = Sonde
  source 1..25: mol_type = other DNA
ggagcctggttctgtcctgcctcaa

SEQ ID NO: 163 - DNA, 25 bp, synthetic construct
  misc_feature 1..25: note = Sonde
  source 1..25: mol_type = other DNA
gactaacaggagcctggttctgtcc

SEQ ID NO: 164 - DNA, 25 bp, synthetic construct
  misc_feature 1..25: note = Sonde
  source 1..25: mol_type = other DNA
acacaaagtcactgctcagtaaact

SEQ ID NO: 165 - DNA, 25 bp, synthetic construct
  misc_feature 1..25: note = Sonde
  source 1..25: mol_type = other DNA
atagggatgatcctgcacgaatggc

SEQ ID NO: 166 - DNA, 25 bp, synthetic construct
  misc_feature 1..25: note = Sonde
  source 1..25: mol_type = other DNA
ggatgatcctgcacgaatggcatgg

SEQ ID NO: 167 - DNA, 25 bp, synthetic construct
  misc_feature 1..25: note = Sonde
  source 1..25: mol_type = other DNA
gccattcgtgcaggatcatccctat

SEQ ID NO: 168 - DNA, 25 bp, synthetic construct
  misc_feature 1..25: note = Sonde
  source 1..25: mol_type = other DNA
ccatgccattcgtgcaggatcatcc

SEQ ID NO: 169 - DNA, 25 bp, synthetic construct
  misc_feature 1..25: note = Sonde
  source 1..25: mol_type = other DNA
agtggacactttttaggatgtctgc

SEQ ID NO: 170 - DNA, 25 bp, synthetic construct
  misc_feature 1..25: note = Sonde
  source 1..25: mol_type = other DNA
gccctgacataagagtttgccagtt

SEQ ID NO: 171 - DNA, 25 bp, synthetic construct
  misc_feature 1..25: note = Sonde
  source 1..25: mol_type = other DNA
cctgtacccacctttcaccagagct

SEQ ID NO: 172 - DNA, 25 bp, synthetic construct
  misc_feature 1..25: note = Sonde
  source 1..25: mol_type = other DNA
gcagacatcctaaaaagtgtccact

SEQ ID NO: 173 - DNA, 25 bp, synthetic construct
  misc_feature 1..25: note = Sonde
  source 1..25: mol_type = other DNA
aactggcaaactcttatgtcagggc

SEQ ID NO: 174 - DNA, 25 bp, synthetic construct
  misc_feature 1..25: note = Sonde
  source 1..25: mol_type = other DNA
agctctggtgaaaggtgggtacagg

SEQ ID NO: 175 - DNA, 25 bp, synthetic construct
  misc_feature 1..25: note = Sonde
  source 1..25: mol_type = other DNA
aattgttggaatttgaaagtggggt

SEQ ID NO: 176 - DNA, 25 bp, synthetic construct
  misc_feature 1..25: note = Sonde
  source 1..25: mol_type = other DNA
accccactttcaaattccaacaatt

SEQ ID NO: 177 - DNA, 25 bp, synthetic construct
  misc_feature 1..25: note = Sonde
  source 1..25: mol_type = other DNA
gtcagcaccgtgcttctctaacttt

SEQ ID NO: 178 - DNA, 25 bp, synthetic construct
  misc_feature 1..25: note = Sonde
  source 1..25: mol_type = other DNA
gcaccgtgcttctctaactttccac

SEQ ID NO: 179 - DNA, 25 bp, synthetic construct
  misc_feature 1..25: note = Sonde
  source 1..25: mol_type = other DNA
cgtgcttctctaactttccacctgc

SEQ ID NO: 180 - DNA, 25 bp, synthetic construct
  misc_feature 1..25: note = Sonde
  source 1..25: mol_type = other DNA
aaagttagagaagcacggtgctgac

SEQ ID NO: 181 - DNA, 25 bp, synthetic construct
  misc_feature 1..25: note = Sonde
  source 1..25: mol_type = other DNA
gtggaaagttagagaagcacggtgc

SEQ ID NO: 182 - DNA, 25 bp, synthetic construct
  misc_feature 1..25: note = Sonde
  source 1..25: mol_type = other DNA
gcaggtggaaagttagagaagcacg

SEQ ID NO: 183 - DNA, 25 bp, synthetic construct
  misc_feature 1..25: note = Sonde
  source 1..25: mol_type = other DNA
cagcctcgcacctaagaacgccgtg

SEQ ID NO: 184 - DNA, 25 bp, synthetic construct
  misc_feature 1..25: note = Sonde
  source 1..25: mol_type = other DNA
cagtgagaaatctgctggggatgcc

SEQ ID NO: 185 - DNA, 25 bp, synthetic construct
  misc_feature 1..25: note = Sonde
  source 1..25: mol_type = other DNA
gaaagggacatacctggcaggtgcc

SEQ ID NO: 186 - DNA, 25 bp, synthetic construct
  misc_feature 1..25: note = Sonde
  source 1..25: mol_type = other DNA
cacggcgttcttaggtgcgaggctg

SEQ ID NO: 187 - DNA, 25 bp, synthetic construct
  misc_feature 1..25: note = Sonde
  source 1..25: mol_type = other DNA
ggcatccccagcagatttctcactg

SEQ ID NO: 188 - DNA, 25 bp, synthetic construct
  misc_feature 1..25: note = Sonde
  source 1..25: mol_type = other DNA
ggcacctgccaggtatgtccctttc

SEQ ID NO: 189 - DNA, 25 bp, synthetic construct
  misc_feature 1..25: note = Sonde
  source 1..25: mol_type = other DNA
ggtagagaccgaggcggatatacag

SEQ ID NO: 190 - DNA, 25 bp, synthetic construct
  misc_feature 1..25: note = Sonde
  source 1..25: mol_type = other DNA
gagaccgaggcggatatacaggcct

SEQ ID NO: 191 - DNA, 25 bp, synthetic construct
  misc_feature 1..25: note = Sonde
  source 1..25: mol_type = other DNA
ctgtatatccgcctcggtctctacc

SEQ ID NO: 192 - DNA, 25 bp, synthetic construct
  misc_feature 1..25: note = Sonde
  source 1..25: mol_type = other DNA
aggcctgtatatccgcctcggtctc

SEQ ID NO: 193 - DNA, 25 bp, synthetic construct
  misc_feature 1..25: note = Sonde
  source 1..25: mol_type = other DNA
tatactggggcccaattctacagac

SEQ ID NO: 194 - DNA, 25 bp, synthetic construct
  misc_feature 1..25: note = Sonde
  source 1..25: mol_type = other DNA
cagacattacttctttgccagttgg

SEQ ID NO: 195 - DNA, 25 bp, synthetic construct
  misc_feature 1..25: note = Sonde
  source 1..25: mol_type = other DNA
gacacattgcaagtctggaagagga

SEQ ID NO: 196 - DNA, 25 bp, synthetic construct
  misc_feature 1..25: note = Sonde
  source 1..25: mol_type = other DNA
gtctgtagaattgggccccagtata

SEQ ID NO: 197 - DNA, 25 bp, synthetic construct
  misc_feature 1..25: note = Sonde
  source 1..25: mol_type = other DNA
gtctgtagaattgggccccagtata

SEQ ID NO: 198 - DNA, 25 bp, synthetic construct
  misc_feature 1..25: note = Sonde
  source 1..25: mol_type = other DNA
tcctcttccagacttgcaatgtgtc

SEQ ID NO: 199 - DNA, 25 bp, synthetic construct
  misc_feature 1..25: note = Sonde
  source 1..25: mol_type = other DNA
catgatcctgggtgaagccatgtgt

SEQ ID NO: 200 - DNA, 25 bp, synthetic construct
  misc_feature 1..25: note = Sonde
  source 1..25: mol_type = other DNA
tgtgtctgaggatgaaaggggatgc

SEQ ID NO: 201 - DNA, 25 bp, synthetic construct
  misc_feature 1..25: note = Sonde
  source 1..25: mol_type = other DNA
cagattgatgtgacatgtggcacct

SEQ ID NO: 202 - DNA, 25 bp, synthetic construct
  misc_feature 1..25: note = Sonde
  source 1..25: mol_type = other DNA
acacatggcttcacccaggatcatg

SEQ ID NO: 203 - DNA, 25 bp, synthetic construct
  misc_feature 1..25: note = Sonde
  source 1..25: mol_type = other DNA
gcatcccctttcatcctcagacaca

SEQ ID NO: 204 - DNA, 25 bp, synthetic construct
  misc_feature 1..25: note = Sonde
  source 1..25: mol_type = other DNA
aggtgccacatgtcacatcaatctg

SEQ ID NO: 205 - DNA, 25 bp, synthetic construct
  misc_feature 1..25: note = Sonde
  source 1..25: mol_type = other DNA
agagggagcacggtcccagtacacc

SEQ ID NO: 206 - DNA, 25 bp, synthetic construct
  misc_feature 1..25: note = Sonde
  source 1..25: mol_type = other DNA
cacggtcccagtacaccttgagtgt

SEQ ID NO: 207 - DNA, 25 bp, synthetic construct
  misc_feature 1..25: note = Sonde
  source 1..25: mol_type = other DNA
tgttacggctgtcccaggaaaggaa

SEQ ID NO: 208 - DNA, 25 bp, synthetic construct
  misc_feature 1..25: note = Sonde
  source 1..25: mol_type = other DNA
ggtgtactgggaccgtgctccctct

SEQ ID NO: 209 - DNA, 25 bp, synthetic construct
  misc_feature 1..25: note = Sonde
  source 1..25: mol_type = other DNA
acactcaaggtgtactgggaccgtg

SEQ ID NO: 210 - DNA, 25 bp, synthetic construct
  misc_feature 1..25: note = Sonde
  source 1..25: mol_type = other DNA
ttcctttcctgggacagccgtaaca

SEQ ID NO: 211 - DNA, 25 bp, synthetic construct
  misc_feature 1..25: note = Sonde
  source 1..25: mol_type = other DNA
actaagagcaacagcctgaggctaa

SEQ ID NO: 212 - DNA, 25 bp, synthetic construct
  misc_feature 1..25: note = Sonde
  source 1..25: mol_type = other DNA
ggctcaccggaaacaggctgaatgt

SEQ ID NO: 213 - DNA, 25 bp, synthetic construct
  misc_feature 1..25: note = Sonde
  source 1..25: mol_type = other DNA
gagacaccagatgaccgcttggtct

SEQ ID NO: 214 - DNA, 25 bp, synthetic construct
  misc_feature 1..25: note = Sonde
  source 1..25: mol_type = other DNA
cagcttccctagaattatacaccag

SEQ ID NO: 215 - DNA, 25 bp, synthetic construct
  misc_feature 1..25: note = Sonde
  source 1..25: mol_type = other DNA
tactgaacaggttacttcaacttgc

SEQ ID NO: 216 - DNA, 25 bp, synthetic construct
  misc_feature 1..25: note = Sonde
  source 1..25: mol_type = other DNA
ttgtaaaaatataaacgtgaggcaa

SEQ ID NO: 217 - DNA, 25 bp, synthetic construct
  misc_feature 1..25: note = Sonde
  source 1..25: mol_type = other DNA
ttagcctcaggctgttgctcttagt

SEQ ID NO: 218 - DNA, 25 bp, synthetic construct
  misc_feature 1..25: note = Sonde
  source 1..25: mol_type = other DNA
acattcagcctgtttccggtgagcc

SEQ ID NO: 219 - DNA, 25 bp, synthetic construct
  misc_feature 1..25: note = Sonde
  source 1..25: mol_type = other DNA
agaccaagcggtcatctggtgtctc

SEQ ID NO: 220 - DNA, 25 bp, synthetic construct
  misc_feature 1..25: note = Sonde
  source 1..25: mol_type = other DNA
ctggtgtataattctagggaagctg

SEQ ID NO: 221 - DNA, 25 bp, synthetic construct
  misc_feature 1..25: note = Sonde
  source 1..25: mol_type = other DNA
gcaagttgaagtaacctgttcagta

SEQ ID NO: 222 - DNA, 25 bp, synthetic construct
  misc_feature 1..25: note = Sonde
  source 1..25: mol_type = other DNA
ttgcctcacgtttatatttttacaa

SEQ ID NO: 223 - DNA, 25 bp, synthetic construct
  misc_feature 1..25: note = Sonde
  source 1..25: mol_type = other DNA
gatgacagttaagaccctaggttgc

SEQ ID NO: 224 - DNA, 25 bp, synthetic construct
  misc_feature 1..25: note = Sonde
  source 1..25: mol_type = other DNA
caatctcaagtctgatgacttgtta

SEQ ID NO: 225 - DNA, 25 bp, synthetic construct
  misc_feature 1..25: note = Sonde
  source 1..25: mol_type = other DNA
agacccatcattgctagcagactat

SEQ ID NO: 226 - DNA, 25 bp, synthetic construct
  misc_feature 1..25: note = Sonde
  source 1..25: mol_type = other DNA
aaggatgggaaatgctcaggtcacg

SEQ ID NO: 227 - DNA, 25 bp, synthetic construct
  misc_feature 1..25: note = Sonde
  source 1..25: mol_type = other DNA
agggctcatccactaaccccctgaa

SEQ ID NO: 228 - DNA, 25 bp, synthetic construct
  misc_feature 1..25: note = Sonde
  source 1..25: mol_type = other DNA
gaaatggatacccttgggttcaact

SEQ ID NO: 229 - DNA, 25 bp, synthetic construct
  misc_feature 1..25: note = Sonde
  source 1..25: mol_type = other DNA
gcaacctagggtcttaactgtcatc

SEQ ID NO: 230 - DNA, 25 bp, synthetic construct
  misc_feature 1..25: note = Sonde
  source 1..25: mol_type = other DNA
taacaagtcatcagacttgagattg

SEQ ID NO: 231 - DNA, 25 bp, synthetic construct
  misc_feature 1..25: note = Sonde
  source 1..25: mol_type = other DNA
atagtctgctagcaatgatgggtct

SEQ ID NO: 232 - DNA, 25 bp, synthetic construct
  misc_feature 1..25: note = Sonde
  source 1..25: mol_type = other DNA
cgtgacctgagcatttcccatcctt

SEQ ID NO: 233 - DNA, 25 bp, synthetic construct
  misc_feature 1..25: note = Sonde
  source 1..25: mol_type = other DNA
ttcagggggttagtggatgagccct

SEQ ID NO: 234 - DNA, 25 bp, synthetic construct
  misc_feature 1..25: note = Sonde
  source 1..25: mol_type = other DNA
agttgaacccaagggtatccatttc

SEQ ID NO: 235 - DNA, 25 bp, synthetic construct
  misc_feature 1..25: note = Sonde
  source 1..25: mol_type = other DNA
gtgccataacgacaattaaattttt

SEQ ID NO: 236 - DNA, 25 bp, synthetic construct
  misc_feature 1..25: note = Sonde
  source 1..25: mol_type = other DNA
agtcttttgttatctatggaggact

SEQ ID NO: 237 - DNA, 24 bp, synthetic construct
  misc_feature 1..24: note = Sonde
  source 1..24: mol_type = other DNA
gttgtgttaaagtttctaattacg

SEQ ID NO: 238 - DNA, 25 bp, synthetic construct
  misc_feature 1..25: note = Sonde
  source 1..25: mol_type = other DNA
gtaactttgggaccaaaacaatgaa

SEQ ID NO: 239 - DNA, 25 bp, synthetic construct
  misc_feature 1..25: note = Sonde
  source 1..25: mol_type = other DNA
tcataagcctactaatccgggatca

SEQ ID NO: 240 - DNA, 25 bp, synthetic construct
  misc_feature 1..25: note = Sonde
  source 1..25: mol_type = other DNA
gggacaagaactaattccacaggag

SEQ ID NO: 241 - DNA, 25 bp, synthetic construct
  misc_feature 1..25: note = Sonde
  source 1..25: mol_type = other DNA
aaaaatttaattgtcgttatggcac

SEQ ID NO: 242 - DNA, 25 bp, synthetic construct
  misc_feature 1..25: note = Sonde
  source 1..25: mol_type = other DNA
agtcctccatagataacaaaagact

SEQ ID NO: 243 - DNA, 25 bp, synthetic construct
  misc_feature 1..25: note = Sonde
  source 1..25: mol_type = other DNA
acgtaattagaaactttaacacaac

SEQ ID NO: 244 - DNA, 25 bp, synthetic construct
  misc_feature 1..25: note = Sonde
  source 1..25: mol_type = other DNA
ttcattgttttggtcccaaagttac

SEQ ID NO: 245 - DNA, 25 bp, synthetic construct
  misc_feature 1..25: note = Sonde
  source 1..25: mol_type = other DNA
tgatcccggattagtaggcttatga

SEQ ID NO: 246 - DNA, 25 bp, synthetic construct
  misc_feature 1..25: note = Sonde
  source 1..25: mol_type = other DNA
ctcctgtggaattagttcttgtccc

SEQ ID NO: 247 - DNA, 25 bp, synthetic construct
  misc_feature 1..25: note = Sonde
  source 1..25: mol_type = other DNA
gccctttcttgaggtctgggtctgc

SEQ ID NO: 248 - DNA, 25 bp, synthetic construct
  misc_feature 1..25: note = Sonde
  source 1..25: mol_type = other DNA
gcagacccagacctcaagaaagggc

SEQ ID NO: 249 - DNA, 25 bp, synthetic construct
  misc_feature 1..25: note = Sonde
  source 1..25: mol_type = other DNA
aggctgtaaccccccttaaactgcc

SEQ ID NO: 250 - DNA, 25 bp, synthetic construct
  misc_feature 1..25: note = Sonde
  source 1..25: mol_type = other DNA
caactatggggaacttaactggagt

SEQ ID NO: 251 - DNA, 25 bp, synthetic construct
  misc_feature 1..25: note = Sonde
  source 1..25: mol_type = other DNA
ggagtcgtttcagatgggtgcttac

SEQ ID NO: 252 - DNA, 24 bp, synthetic construct
  misc_feature 1..24: note = Sonde
  source 1..24: mol_type = other DNA
ggcagtttaaggggggttacagcc

SEQ ID NO: 253 - DNA, 25 bp, synthetic construct
  misc_feature 1..25: note = Sonde
  source 1..25: mol_type = other DNA
actccagttaagttccccatagttg

SEQ ID NO: 254 - DNA, 25 bp, synthetic construct
  misc_feature 1..25: note = Sonde
  source 1..25: mol_type = other DNA
gtaagcacccatctgaaacgactcc

SEQ ID NO: 255 - DNA, 25 bp, synthetic construct
  misc_feature 1..25: note = Sonde
  source 1..25: mol_type = other DNA
gaagagtttcagcccttcagacaac

SEQ ID NO: 256 - DNA, 25 bp, synthetic construct
  misc_feature 1..25: note = Sonde
  source 1..25: mol_type = other DNA
gatccaggtttgtcacgcaagctga

SEQ ID NO: 257 - DNA, 25 bp, synthetic construct
  misc_feature 1..25: note = Sonde
  source 1..25: mol_type = other DNA
ccgagtgggcacatcaagcacagtg

SEQ ID NO: 258 - DNA, 25 bp, synthetic construct
  misc_feature 1..25: note = Sonde
  source 1..25: mol_type = other DNA
gttgtctgaagggctgaaactcttc

SEQ ID NO: 259 - DNA, 25 bp, synthetic construct
  misc_feature 1..25: note = Sonde
  source 1..25: mol_type = other DNA
tcagcttgcgtgacaaacctggatc

SEQ ID NO: 260 - DNA, 25 bp, synthetic construct
  misc_feature 1..25: note = Sonde
  source 1..25: mol_type = other DNA
cactgtgcttgatgtgcccactcgg

SEQ ID NO: 261 - DNA, 25 bp, synthetic construct
  misc_feature 1..25: note = Sonde
  source 1..25: mol_type = other DNA
cacaggtcttgccgagacccccacg

SEQ ID NO: 262 - DNA, 25 bp, synthetic construct
  misc_feature 1..25: note = Sonde
  source 1..25: mol_type = other DNA
ccacgggctcactgttcagctcatc

SEQ ID NO: 263 - DNA, 25 bp, synthetic construct
  misc_feature 1..25: note = Sonde
  source 1..25: mol_type = other DNA
gctctgtcacagtttcccacgactt

SEQ ID NO: 264 - DNA, 25 bp, synthetic construct
  misc_feature 1..25: note = Sonde
  source 1..25: mol_type = other DNA
cgtgggggtctcggcaagacctgtg

SEQ ID NO: 265 - DNA, 25 bp, synthetic construct
  misc_feature 1..25: note = Sonde
  source 1..25: mol_type = other DNA
gatgagctgaacagtgagcccgtgg

SEQ ID NO: 266 - DNA, 25 bp, synthetic construct
  misc_feature 1..25: note = Sonde
  source 1..25: mol_type = other DNA
aagtcgtgggaaactgtgacagagc

SEQ ID NO: 267 - DNA, 25 bp, synthetic construct
  misc_feature 1..25: note = Sonde
  source 1..25: mol_type = other DNA
ggccaaattgtgccacccctcccaa

SEQ ID NO: 268 - DNA, 25 bp, synthetic construct
  misc_feature 1..25: note = Sonde
  source 1..25: mol_type = other DNA
aattcccaggacctcctaatatggc

SEQ ID NO: 269 - DNA, 25 bp, synthetic construct
  misc_feature 1..25: note = Sonde
  source 1..25: mol_type = other DNA
ggtcgttgtaggcccagcacagtgg

SEQ ID NO: 270 - DNA, 25 bp, synthetic construct
  misc_feature 1..25: note = Sonde
  source 1..25: mol_type = other DNA
ttgggaggggtggcacaatttggcc

SEQ ID NO: 271 - DNA, 25 bp, synthetic construct
  misc_feature 1..25: note = Sonde
  source 1..25: mol_type = other DNA
gccatattaggaggtcctgggaatt

SEQ ID NO: 272 - DNA, 25 bp, synthetic construct
  misc_feature 1..25: note = Sonde
  source 1..25: mol_type = other DNA
ccactgtgctgggcctacaacgacc

SEQ ID NO: 273 - DNA, 25 bp, synthetic construct
  misc_feature 1..25: note = Sonde
  source 1..25: mol_type = other DNA
cctagccatgagccaattccttgca

SEQ ID NO: 274 - DNA, 25 bp, synthetic construct
  misc_feature 1..25: note = Sonde
  source 1..25: mol_type = other DNA
tgcaaggaattggctcatggctagg

(19) United States
(12) Patent Application Publication
(10) Pub. No.: US 2024/0417799 A1
(43) Pub. Date: Dec. 19, 2024

(54) METHOD FOR DETERMINING IN VITRO OR EX VIVO THE IMMUNE STATUS OF AN INDIVIDUAL

(71) Applicant: BIOMERIEUX, Marcy l'Etoile (FR)

(72) Inventors: Marine MOMMERT, Lyon (FR); Olivier TABONE, Lyon (FR); Julien TEXTORIS, Villeurbanne (FR); François MALLET, Villeurbanne (FR)

(73) Assignee: BIOMERIEUX, Marcy l'Etoile (FR)

(21) Appl. No.: 18/772,821

(22) Filed: Jul. 15, 2024

Related U.S. Application Data
(62) Division of application No. 17/256,811, filed on Dec. 29, 2020, now Pat. No. 12,071,670.

(30) Foreign Application Priority Data
Jun. 29, 2018 (EP) 18180991.4
Sep. 12, 2018 (FR) 18/58167

(51) Int. Cl. C12Q 1/6883

(52) U.S. Cl. CPC C12Q 1/6883; C12Q 2600/158

(57) ABSTRACT

A method for determining in vitro or ex vivo the immune status of an individual, preferably a patient, including a step of detecting and/or quantifying the expression of one or more HERV/MaLR sequences in a biological sample of the individual. Also relates to the tools for implementing the method and to the uses thereof.

### CROSS-REFERENCE TO RELATED APPLICATIONS

This is a Division of application Ser. No. 17/256,811 filed Dec. 29, 2020, which in turn is a National Stage Entry of PCT/EP2019/067330 filed Jun. 28, 2019, which claims priority to FR 18/58167 filed Sep. 12, 2018, and EP 18180991.4 filed Jun. 29, 2018. The disclosure of the prior applications is hereby incorporated by reference herein in its entirety.

### REFERENCE TO AN ELECTRONIC SEQUENCE LISTING

The present application contains a Sequence Listing that has been submitted electronically and is hereby incorporated by reference in its entirety. The electronic Sequence Listing is named Sequence_Listing_ST26.xml, was created on Jul. 11, 2024, and is 358,852 bytes in size.

The present invention relates to a method for determining in vitro or ex vivo the immune status of an individual, preferably a patient, comprising a step of detecting and/or quantifying the expression, in a biological sample of said individual, of one or several sequences of HERV/MaLR, as well as the tools for implementing it and the uses thereof.

The immune system is a system for defending the body against what is recognized as non-self, such as pathogens (e.g. viruses, bacteria, parasites). In mammals, there are two main types of mechanisms: a non-specific defense mechanism, also called «innate» or «natural» immunity, and a specific defense mechanism, also called «acquired» or «adaptive» immunity.

These immune responses require very fine regulation. In a healthy individual, the immune response will be qualified as «normal» (we can also speak of immune status of immunocompetence). However, the immune response can sometimes be impaired. We will talk about inflammation status or hyperactive immune status when the immune system is more active than normal, as in the case of inflammatory or autoimmune diseases. In autoimmune diseases, the immune system of the body triggers an inflammatory response with characterized immunization against antigens of the self. Conversely, we will talk about immunosuppression status (or immunodepression or immunodeficiency or hypoactive immune status or immune paralysis), when the immune system is less active than normal.

The immunosuppression can have various origins, take many forms, and affect innate immunity and/or adaptive immunity. Particularly, the sepsis is a major public health problem, which is the leading cause of death in intensive care units. It is estimated that 28 million people develop sepsis each year worldwide, among which 8million will die of the pathology (Fleischmann et al. (2016) American journal of respiratory and critical care medicine; 193(3): 259-72). In a patient with sepsis (also known as in a septic state), the immune response is deregulated, following an infection, leading to multiple and potentially fatal organ failure and dysfunction. This immune response is complex and evolves over time, with excessive pro-inflammatory and anti-inflammatory phenomena which may be concomitant. All of these immune system disorders lead to organ failure, immune system paralysis, and secondary infections. The septic shock is a subtype of sepsis, in which hypotension persists despite adequate vascular filling (Singer et al (2016) JAMA; 315 (8): 801-810). In the initial stage of sepsis, an inflammatory or even hyper-inflammatory response seems to predominate, which is the cause of tissue damage and organ failure, in particular in the kidney. This is why clinical trials in the field of sepsis have long focused on anti-inflammatory treatments, but with inconclusive results. More recent studies on the pathophysiology of sepsis have shown that an anti-inflammatory or immunosuppression response occurs in some patients in septic state, either concomitantly with the initial inflammation or later. The patient may then be in a state of immunosuppression, which may potentially be severe, depending on the respective degrees of pro-inflammatory and anti-inflammatory responses. These immunodepressed patients present a high risk of developing nosocomial infections (or HAI, Health-care Associated Infections), and could advantageously benefit from immunostimulatory treatments.

It therefore appears important to be able to determine the immune status of an individual, and particularly to be able to identify an immunosuppression status, in order to be able to adapt the therapeutic management. Yet, individuals with immune system disorders do not present specific clinical signs. There is therefore a significant need for the identification of biomarkers, which make it possible to determine the immune status of an individual.

Currently, the reference test for monitoring immune alterations in intensive care patients (e.g. patients with sepsis, trauma, major surgery, burns, or patients with pancreatitis) is the decreased expression of the HLA-DR (human leukocyte antigen-D related) on the surface of monocytes (mHLA-DR), measured by flow cytometry. Indeed, this marker provides valuable information in terms of prediction of mortality or the assessment of the secondary infections risk in these patients. The HLA-DR is a surface receptor belonging to the MHC (major histocompatibility complex) class II. In particular, the measurement of the mHLA-DR expression represents the gold standard for identifying whether a patient with sepsis is immunodepressed or not (Monneret and Venet (2016) Cytometry Part B (Clinical Cytometry) 90B: 376-386). However, this approach requires extensive pre-analytical sample manipulation (Monneret and Venet (2014) HLA-DR monocyte in sepsis: shall we stop following the flow? Crit Care 18:102). Moreover, the accessibility to a flow cytometer is not always possible in all hospitals, and the measurement is difficult to standardize from one hospital to another, or even from one operator to another.

To overcome these drawbacks, other biomarkers, using molecular biology tools, have been proposed, such as for example a biomarker based on the ratio of the expression level, at the mRNA level, of CD74 on day D3 (following admission of the patient within a medical facility) on the CD74 expression level on day D1. The CD74 represents the invariant chain y of HLA-DR. The CD74 D3/D1 expression ratio has been demonstrated to be associated with the onset of secondary infections acquired in intensive care (Peronnet et al (2017) Intensive Care Medicine; 43(7): 1013-20). The patent application WO2012/101387 describes a method for determining the immune status of an individual, from the analysis of the expression of at least two genes selected from several groups of genes. It has also been proposed, in the patent application WO2013/156627, a method for determining the immunodepressed or non-immunodepressed status, from the determination of the anellovirus load, in a biological sample. However, none of these biomarkers have yet come to replace the use of mHLA-DR. These biomarkers have the particular drawback of not making it possible to identify in which phase the patient is (i.e. inflammatory phase versus immunosuppressive phase), their objective being primarily to be able to identify immunodepressed patients, for whom it would be relevant to administer immunostimulant treatments.

At the date of the present invention, it therefore remains necessary to find new biomarkers, which make it possible to determine the immune status of an individual.

The endogenous retroviruses, or ERVs (for Endogenous Retro Virus) designate stable sequences of the genome of an organism and having structural analogies with certain infectious exogenous retroviruses (including the presence of two LTRs, or Long Terminal Repeats, which surround the genes encoding for putative proteins). Their origin is uncertain, but the most likely hypothesis is that of germ cell infection with a retrovirus. Following mutations in the retrovirus which would have made it defective, the infected germ cells could have survived, and the genome of the retrovirus, integrated into the genome of the organism, could have been transmitted to the next generation, and persist in the offspring within the genome of the organism.

In humans, HERVs (Human Endogenous RetroViruses) have only been demonstrated since the sequencing of the human genome. Together with MaLRs (Mammalian apparent LTR-Retrotransposons), which have a similar structure to HERVs, they represent 8.3% of the human genome, with a number of over 400,000 elements. By comparison, the 30,000 to 40,000 genes encoding proteins represent only 2% of human DNA. HERVs are subdivided into three major classes (I, II and III) and several groups (sometimes called «families» in the present patent application). HERVs are retroelements that are transposed only by a copy and paste mode, through an RNA intermediate and reverse transcriptase. They have long been considered as «junk DNA». While they may be inactive due to mutations or through epigenetic mechanisms, their role begins to appear, both in physiological and pathological contexts. Thus, it has been shown that HERV-W participates in one of the mechanisms ensuring the formation of the placenta. The HERV-K superfamily is the most studied in relation to carcinogenesis. The expression of certain HERVs has also been described in certain autoimmune diseases, such as multiple sclerosis or lupus erythematosus, and in interferonopathies, without any suggestion of a possible link between reactivation of HERVs and the immune status.

Thus, it has never been described or suggested that analyzing the expression of HERV in humans could be useful to determine the immune status of an individual. Furthermore, the reactivation of HERV has never been described in the pathology of sepsis.

Yet, it was discovered that, completely unexpectedly, among the approximately 420,000 existing HERV/MaLR, the analysis of the expression of some of them made it possible to determine the immune status of an individual.

Thus, the present invention relates to a method for determining in vitro or ex vivo the immune status of an individual, preferably a patient, comprising a step of detecting and/or quantifying the expression, in a biological sample of said individual (or biological test sample), of at least a part, preferably of a size of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 nucleotides, of at least one HERV/MaLR sequence selected from the sequences identified in SEQ ID NO: 1 to 34 or from the sequences which exhibit at least 99%, preferably at least 99.1%, preferably at least 99.2%, preferably at least 99.3%, preferably at least 99.4%, preferably at least 99.5%, preferably at least 99.6%, preferably at least 99.7%, preferably at least 99.8%, preferably at least 99.9% of identity with one of the sequences identified in SEQ ID NO: 1 to 34, from the following lists:

TABLE 1
List 1

| | | | HERV/ |
|---|---|---|---|
| SEQ | | Name of the probeset | MaLR |
| ID | | corresponding to the | group |
| NO: | GRCh38 Location | HERV-V3 chip | name |
| 1 | chr19:54891074-54891496 | 190665001-HERV0376 | HERV0376 |
| 2 | chr22:36153696-36154283 | 220247002-HERV0797 | HERV0797 |
| 3 | chr17:35505737-35508365 | 170369402HE41env | HERV-E41 |
| 4 | chr12:112971073- | 121601801-HERV0492 | HERV0492 |
| | 112971451 | | |
| 5 | chr1:78648318-78648697 | 011052702-MALR1044 | MALR1044 |
| 6 | chr1:78623489-78623954 | 011052202-HERV1033 | HERV1033 |
| 7 | chr13:42884951-42886257 | 130360601-HERV0808 | HERV0808 |
| 8 | chr14:91230494-91230820 | 141107102-MALR1019 | MALR1019 |
| 9 | chr2:102363654- | 021460102- | HERV0599 |
| | 102366601 | HERV0599uL | |
| 10 | chr2:102013616- | 021456001- | MALR1017 |
| | 102013971 | MALR1017uL | |
| 11 | chr5:14551189-14551685 | 050286701-HERV0513 | HERV0513 |
| 12 | chr5:14562791-14563322 | 050287402-MALR1022 | MALR1022 |

TABLE 2
List 2

| SEQ | | Name of the probeset | HERV/ |
|---|---|---|---|
| ID | | corresponding to the | MaLR |
| NO: | GRCh38 Location | HERV-V3 chip | group name |
| 13 | chr5:132453630-132454148 | 052182701-MALR1129 | MALR1129 |
| 14 | chr19:41812466-41813010 | 190478501-MALR1003 | MALR1003 |
| 15 | chr1:155637287-155637547 | 011790601ERV9sLU5 | ERV9 |
| 16 | chr5:170290289-170290812 | 052681601-MALR1018 | MALR1018 |
| 17 | chr16:50662453-50662912 | 160627301-MALR1014 | MALR1014 |
| 18 | chr11:122671887-122672147 | 111686702-HERV0861 | HERV0861 |
| 8 | chr14:91230494-91230820 | 141107102-MALR1019 | MALR1019 |
| 19 | chr4:15825146-15825565 | 040318302-MALR1134 | MALR1134 |
| 20 | chr4:83464568-83464963 | 041529101-MALR1026 | MALR1026 |
| 21 | chr14:91222760-91223118 | 141106902-MALR1133 | MALR1133 |

TABLE 3
List 3

| SEQ | | Name of the probeset | HERV/ |
|---|---|---|---|
| ID | | corresponding to the | MaLR |
| NO: | GRCh38 Location | HERV-V3 chip | group name |
| 1 | chr19:54891074-54891496 | 190665001-HERV0376 | HERV0376 |
| 1 | chr19:54891074-54891496 | 190665002-HERV0376 | HERV0376 |
| 22 | chr6:18403673-18404108 | 060281701-MALR1043 | MALR1043 |
| 23 | chr4:184850413-184850785 | 043166601-MALR1018 | MALR1018 |
| 24 | chr10:5856198-5856795 | 100090601-HERV0429 | HERV0429 |
| 25 | chr6:107800650-107801138 | 061529601-HERV0492 | HERV0492 |
| 26 | chr10:60410534-60411224 | 100871501-MALR1020 | MALR1020 |
| 27 | chr17:78345106-78345577 | 170842002-MALR1003 | MALR1003 |

TABLE 4
List 4

| SEQ | | Name of the probeset | HERV/ |
|---|---|---|---|
| ID | | corresponding to the | MaLR |
| NO: | GRCh38 Location | HERV-V3 chip | group name |
| 28 | chr8:125945973-125951030 | 081921103-HERV0958 | HERV0958 |
| 29 | chr3:167401329-167401866 | 032622601MR41sLU5p | MR41 |
| 30 | chr22:36147793-36148208 | 220246901-HERV0889 | HERV0889 |
| 31 | chr6:127790579-127792191 | 061827101-HERV0856 | HERV0856 |
| 3 | chr17:35505737-35508365 | 170369402HE41env | HERV-E41 |
| 32 | chr17:77462942-77463350 | 170828901-HERV0770 | HERV0770 |
| 28 | chr8:125945973-125951030 | 081921101-HERV0958 | HERV0958 |
| 28 | chr8:125945973-125951030 | 081921102-HERV0958 | HERV0958 |
| 33 | chr19:14612123-14612747 | 190148802-MALR1127 | MALR1127 |
| 34 | chr12:9038254-9038598 | 120093401-HERV1034 | HERV1034 |

In the context of the present invention:
«determining the immune status» of an individual means evaluating the capacity of the body to implement an immune response and to defend against attacks or infections. The immune status can in particular be determined as being a normal immune status (or immunocompetence status), an inflammation status (or hyperactive immune status) when the immune system is more active than normal, or an immunosuppression status (or immunodepression or immunodeficiency or hypoactive immune status or immune paralysis), when the immune system is less active than normal.
The term «HERV/MaLR» means the elements of the HERV and MaLR type, as presented in the introduction. The abbreviated term «HERV» may also sometimes be used, and have the same meaning as «HERV/MaLR». In the literature, many aliases have been used to describe the same HERV element or the same group (or family) of HERV, and there is still a need for standardization to this day. In the present application, to avoid any confusion, reference will firstly be made, in order to determine the identity of a HERV element, to the chromosomal location of said HERV element, more particularly on the basis of the GRCh38 (Genome Reference Consortium Human Build 38). We can also sometimes refer to the different probesets and the different probes of the HERV-V3 chip targeting said HERV element. It should also be noted that, for a given HERV element, as identified by its GRCh38 location, the sequences found in different individuals may differ from the sequence indicated in the GRCh38 base, due to polymorphism (Wildschutte et al (2016), Discovery of unfixed endogenous retrovirus insertions in diverse human populations, PNAS 113 (16): E2326-34).
The term «individual» designates a human being, whatever his state of health. A «healthy individual» within the meaning of the present invention is an individual who does not exhibit any dysregulation of the immune system. The term «patient» refers to an individual who has come into contact with a healthcare professional, such as a physician (for example, a general practitioner) or medical facility (for example, a hospital emergency or resuscitation service or intensive care unit)
The term «detection of the expression of a sequence» means the demonstration of the expression of said sequence, without necessarily a quantitative measurement. In the case of an mRNA transcript, the detection can be carried out by a direct method, by any method known to one skilled in the art making it possible to determine the presence of said transcript in a sample, or by indirect detection of the transcript after transformation of said transcript into DNA, or after amplification of said transcript or after amplification of the DNA obtained after transformation of said transcript into DNA. The «quantification of the expression of a sequence» refers to the evaluation of the expression level of the sequence, quantitatively. Many methods exist for the detection of nucleic acids (see, for example, Kricka et al., Clinical Chemistry, 1999, No. 45 (4), p. 453-458; Relier G H et al., DNA Probes, 2nd Ed., Stockton Press, 1993, sections 5 and 6, p. 173-249).
The term «biological sample» refers to any sample from an individual, which can be of various natures, such as blood, serum, plasma, sputum, urine, stool, skin, cerebrospinal fluid, bronchoalveolar lavage fluid, saliva, gastric secretions, semen, seminal fluid, tears, spinal cord, trigeminal nerve ganglion, adipose tissue, lymphoid tissue, placental tissue, tissue of the gastrointestinal tract, tissue of the genital tract, tissue of the central nervous system. Particularly, this sample can be a biological fluid, preferably selected from the whole blood (as collected from the venous route, that is to say containing white and red cells, platelets and plasma), plasma and serum. It can also be any type of cells extracted from a blood sample, such as peripheral blood mononuclear cells (or PBMCs), B cell subpopulations, purified monocytes, or neutrophils.
In order to determine the «percentage of sequence identity» of one nucleic acid sequence with another nucleic acid sequence, the two sequences are first aligned optimally. The two sequences to be compared can be of the same size or of different sizes. In some cases, it may be necessary to introduce «holes» in one of the sequences, in order to allow optimal alignment with the second sequence. The optimal alignment of the sequences can in particular be carried out by using the algorithm of Smith and Waterman (J. Theor. Biol., 91 (2): 370-380, 1981), the algorithm of Needleman and Wunsch (J. Mol. Biol, 48 (3): 443-453, 1972), or the method of Pearson and Lipman (Proc. Natl. Acad. Sri. USA, 85 (5): 2444-2448, 1988). Some software allow to implementing some of these algorithms, such as GAP, BESTFIT, FASTA, TFASTA (Wisconsin Genetics Software Package Release 7.0, Genetic Computer Group, 575, Science Drive, Madison, Wisconsin), BLAST or even CLUSTALW (Nucleic Acids Res. 1994 Nov. 11; 22 (22): 4673-80. CLUSTAL W: improving the sensitivity of progressive multiple sequence alignment through sequence weighting, position-specific gap penalties and weight matrix choice). The best alignment (i.e. the one allowing to obtain the highest percentage of identity on the comparison window), among those generated by these various methods, is selected. The respective nucleotides located at the same position of each of the sequences are then compared. When a given position is occupied by the same nucleotide in both sequences, then the sequences are identical for that position. The percentage of sequence identity is then determined as a function of the number of positions for which the respective nucleotides are identical, relative to the total number of nucleotides of the positions for which the alignment has been possible, at the level of the comparison window.
A «biomarker» or «marker» is an objectively measurable biological characteristic which represents an indicator of normal or pathological biological processes or of pharmacological response to a therapeutic intervention. It may particularly be a molecular biomarker, preferably detectable at the mRNA level. More particularly, the biomarker can be an endogenous biomarker or loci (such as a HERV or a gene, which are found in the chromosomal material of an individual) or an exogenous biomarker (such as a virus).
The «sepsis» is a disease in which the immune response is deregulated in an individual, following an infection, leading to multiple organ failure and dysfunction and potentially fatal. The «septic shock» is a subtype of sepsis, in which hypotension persists despite adequate blood supply.
The term «amplification primer» means a nucleotide fragment which may comprise from 5 to 100 nucleotides, preferably from 15 to 30 nucleotides, and having a specificity of hybridization with a target nucleotide sequence, under conditions determined for the initiation of an enzymatic polymerization, for example in an enzymatic amplification reaction of the target nucleotide sequence. Generally, «pairs of primers», consisting of two primers, are used. When it is desired to carry out the amplification of several different HERVs, several different pairs of primers are preferably used, each preferably having an ability to specifically hybridize with a different HERV.
The term «hybridization probe» means a nucleotide fragment typically comprising from 5 to 100 nucleotides, preferably from 15 to 90 nucleotides, even more preferably from 15 to 35 nucleotides, having a specificity of hybridization under conditions determined to form a hybridization complex with a target nucleotide sequence. The probe also includes a reporter (such as a fluorophore, an enzyme or any other detection system), which will allow the detection of the target nucleotide sequence. In the present invention, the target nucleotide sequence can be a nucleotide sequence included in a messenger RNA (mRNA) or a nucleotide sequence comprised in a complementary DNA (cDNA) obtained by reverse transcription of said mRNA. When it is desired to target several different HERVs, several different probes are preferably used, each preferably having an ability to specifically hybridize with a different HERV.
The term «hybridization» means the process during which, under appropriate conditions, two nucleotide fragments, such as for example a hybridization probe and a target nucleotide fragment, having sufficiently complementary sequences, are capable of forming a double strand with stable and specific hydrogen bonds. A nucleotide fragment «capable of being hybridized» with a polynucleotide is a fragment capable of being hybridized with said polynucleotide under hybridization conditions, which can be determined in each case in a known manner. The hybridization conditions are determined by the stringency, that is to say the stringency of the operating conditions. The hybridization is all the more specific as it is carried out at higher stringency. The stringency is defined in particular as a function of the base composition of a probe/target duplex, as well as by the degree of mismatch between two nucleic acids. The stringency can also be a function of the reaction parameters, such as the concentration and the type of ionic species present in the hybridization solution, the nature and the concentration of denaturing agents and/or the hybridization temperature. The stringency of the conditions under which a hybridization reaction is to be performed will depend primarily on the used hybridization probes. All of these data are well known and the appropriate conditions can be determined by one skilled in the art. In general, depending on the length of the used hybridization probes, the temperature for the hybridization reaction is comprised between about 20 and 70° C., particularly between 35 and 65° C. in saline solution at a concentration of about 0.5 to 1 M. A step of detecting the hybridization reaction is then carried out.
The term «enzymatic amplification reaction» means a process generating multiple copies of a target nucleotide fragment, by the action of at least one enzyme. Such amplification reactions are well known to one skilled in the art and the following techniques may be mentioned in particular: PCR (Polymerase Chain Reaction), LCR (Ligase Chain Reaction), RCR (Repair Chain Reaction), 3SR (Self Sustained Sequence Replication) with the patent application WO-A-90/06995, NASBA (Nucleic Acid Sequence-Based Amplification), TMA (Transcription Mediated Amplification) with U.S. Pat. No. 5,399,491, and LAMP (Loop mediated isothermal amplification) with the U.S. Pat. No. 6,410,278. When the enzymatic amplification reaction is a PCR, we will speak more particularly of RT-PCR (RT for «reverse transcription»), when the amplification step is preceded by a messenger RNA (mRNA) reverse-transcription step into complementary DNA (cDNA), and from qPCR or RT-qPCR when PCR is quantitative.

To the knowledge of the inventors, it has never been described or suggested that the detection and/or the quantification of the expression of HERV/MaLR could make it possible to determine the immune status of an individual. Particularly, the involvement of HERV/MaLR has never been described in the field of sepsis.

All of the HERV/MaLRs of SEQ ID NO: 1 to 34 have been identified among the approximately 420,000 HERV/MaLRs in the genome. These are HERV/MaLR that we can target with the HERV-V3 chip (Becker et al, BMC Genomics. 2017; 18:286). More particularly, these HERV/MaLR are expressed in the data sets used in the Examples. Even more particularly, these expressed HERV/MaLR are modulated between the conditions of interest retained in the Examples.

The detection and/or the quantification of the expression of the HERVs according to the present invention, to determine the immune status of an individual, can be carried out by means of molecular tools which have advantages relative to the use of flow cytometry for the measurement of mHLA-DR, in terms of accessibility within hospitals and standardization. Little sample manipulation is required and the results are easy to interpret. Moreover, the expression of HERVs according to the present invention can be detected and/or quantified on several types of platforms, such as DNA chips or by PCR, in order to determine the immune status. Some of the HERVs according to the present invention can be detected early, from D1, while the measurement of mHLA-DR is carried out on D3 (Monneret and Venet (2014) Monocyte HLA-DR in sepsis: shall we stop following the flow? Crit Care 18:102).

Preferably, the present invention relates to a method for determining in vitro or ex vivo the immune status of an individual, preferably a patient, comprising:
a step of detecting and/or quantifying the expression, in a biological sample of said individual (or test biological sample), of at least part of at least one HERV/MaLR sequence selected from the sequences identified in SEQ ID NO: 1 to 34 or from the sequences which have at least 99% identity with one of the sequences identified in SEQ ID NO: 1 to 34, from Lists 1 to 4, previously described;
a step in which the expression in the test biological sample is compared with a reference expression, or with the expression in a reference biological sample;
a step in which the immune status of the individual is determined from this comparison.

The reference biological sample can be of various natures, but it is preferably identical in nature, or at least similar in nature, to the nature of the test biological sample. For example, if the test biological sample is a whole blood sample, the reference biological sample will preferably be a whole blood sample, or possibly a plasma or serum sample. The biological reference sample can be a «natural» sample, that is to say from an individual whose immune status is known or determined according to a reference method (for example, by the mHLA-DR method). For example, it can be from an individual with an immune status known to be an immunocompetent status, an inflammation status, or an immunosuppression status. Preferably, if the test biological sample is from a human, the reference biological sample is also from a human. Even more preferably, the reference biological sample comes from the same individual as that from which the test biological sample comes. The reference biological sample can also be a «synthetic» sample, that is to say a sample containing a calibrated amount of at least one of the sequences SEQ ID NO: 1 to 34.

Preferably, the invention relates to a method for determining the immune status of an individual, as described above, in which the expression of at least 2 different sequences selected from the sequences identified in SEQ. Nos: 1 to 34 or from the sequences which exhibit at least 99% identity with one of the sequences identified in SEQ ID NO: 1 to 34 is detected and/or quantified. The lists 1 to 4 being complementary to each other, these at least two different sequences are preferably selected in two different lists.

Preferably, the invention relates to a method for determining the immune status of an individual, as described above, in which the expression of at least 3 different sequences selected from the sequences identified in SEQ ID NO: 1 to 34 or from the sequences which exhibit at least 99% identity with one of the sequences identified in SEQ ID NO: 1 to 34 is detected and/or quantified. These at least three different sequences are preferably selected from two different lists, more preferably from three different lists.

Preferably, the invention relates to a method for determining the immune status of an individual, as described above, in which the expression of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34 different sequences selected from the sequences identified in SEQ ID NO: 1 to 34 or from the sequences which exhibit at least 99% identity with one of the sequences identified in SEQ ID NO: 1 to 34 is detected and/or quantified. These at least 4, at least 5, at least 6, at least 7, at least 8, at least 9, at least 10, at least 11, at least 12, at least 13, at least 14, at least 15, at least 16, at least 17, at least 18, at least 19, at at least 20, at least 21, at least 22, at least 23, at least 24, at least 25, at least 26, at least 27, at least 28, at least 29, at least 30, at least 31, at least 32, at least 33, at least 34 different sequences are preferably selected from two different lists, more preferably from three different lists, even more preferably from four different lists.

The HERVs of SEQ ID NO: 1, 3 and 8 were identified from two different strategies, as described in the Examples, and are thus found in two Lists respectively. Moreover, the inventors assigned a rating for the different sequences of HERV, as explained in Examples 4 to 6. In Example 7, the HERVs were ranked in order of importance. Also, quite preferably, the invention relates to a method for determining the immune status of an individual, as described above, in which the expression of at least 1, at least 2, at least 3, at least 4, at least 5, at least 6, at least 7 different sequences selected from the sequences identified in SEQ ID NO: 1, SEQ ID NO: 3, SEQ ID NO: 8, SEQ ID NO: 11, SEQ ID NO: 12, SEQ ID NO: 13 and SEQ ID NO: 28, or from the sequences which exhibit at least 99%, at least 99.1%, at least 99.2%, at least 99.3%, at least 99.4%, at least 99.5%, at least 99.6%, at least 99.7%, at least 99.8%, at least 99.9% identity with one of the sequences SEQ ID NO: 1, 3, 8, 11, 12, 13 and 28 is detected and/or quantified. Preferred combinations of at least 2 corresponding HERVs are listed in Table 5 below.

TABLE 5

| Number | |
|---|---|
| of | |
| HERV | |
| in the | |
| com- | |
| bination | Preferred combinations of at least 2 HERVs |
| 2 | SEQ ID NO: 1, SEQ ID NO: 3 |
| | SEQ ID NO: 1, SEQ ID NO: 8 |
| | SEQ ID NO: 1, SEQ ID NO: 11 |
| | SEQ ID NO: 1, SEQ ID NO: 12 |
| | SEQ ID NO: 1, SEQ ID NO: 13 |
| | SEQ ID NO: 1, SEQ ID NO: 28 |
| | SEQ ID NO: 3, SEQ ID NO: 8 |
| | SEQ ID NO: 3, SEQ ID NO: 11 |
| | SEQ ID NO: 3, SEQ ID NO: 12 |
| | SEQ ID NO: 3, SEQ ID NO: 13 |
| | SEQ ID NO: 3, SEQ ID NO: 28 |
| | SEQ ID NO: 8, SEQ ID NO: 11 |
| | SEQ ID NO: 8, SEQ ID NO: 12 |
| | SEQ ID NO: 8, SEQ ID NO: 13 |
| | SEQ ID NO: 8, SEQ ID NO: 28 |
| | SEQ ID NO: 11, SEQ ID NO: 12 |
| | SEQ ID NO: 11, SEQ ID NO: 13 |
| | SEQ ID NO: 11, SEQ ID NO: 28 |
| | SEQ ID NO: 12, SEQ ID NO: 13 |
| | SEQ ID NO: 12, SEQ ID NO: 28 |
| | SEQ ID NO: 13, SEQ ID NO: 28 |
| 3 | SEQ ID NO: 1, SEQ ID NO: 3, SEQ ID NO: 8 |
| | SEQ ID NO: 1, SEQ ID NO: 3, SEQ ID NO: 11 |
| | SEQ ID NO: 1, SEQ ID NO: 3, SEQ ID NO: 12 |
| | SEQ ID NO: 1, SEQ ID NO: 3, SEQ ID NO: 13 |
| | SEQ ID NO: 1, SEQ ID NO: 3, SEQ ID NO: 28 |
| | SEQ ID NO: 1, SEQ ID NO: 8, SEQ ID NO: 11 |
| | SEQ ID NO: 1, SEQ ID NO: 8, SEQ ID NO: 12 |
| | SEQ ID NO: 1, SEQ ID NO: 8, SEQ ID NO: 13 |
| | SEQ ID NO: 1, SEQ ID NO: 8, SEQ ID NO: 28 |
| | SEQ ID NO: 1, SEQ ID NO: 11, SEQ ID NO: 12 |
| | SEQ ID NO: 1, SEQ ID NO: 11, SEQ ID NO: 13 |
| | SEQ ID NO: 1, SEQ ID NO: 11, SEQ ID NO: 28 |
| | SEQ ID NO: 1, SEQ ID NO: 12, SEQ ID NO: 13 |
| | SEQ ID NO: 1, SEQ ID NO: 12, SEQ ID NO: 28 |
| | SEQ ID NO: 1, SEQ ID NO: 13, SEQ ID NO: 28 |
| | SEQ ID NO: 3, SEQ ID NO: 8, SEQ ID NO: 11 |
| | SEQ ID NO: 3, SEQ ID NO: 8, SEQ ID NO: 12 |
| | SEQ ID NO: 3, SEQ ID NO: 8, SEQ ID NO: 13 |
| | SEQ ID NO: 3, SEQ ID NO: 8, SEQ ID NO: 28 |
| | SEQ ID NO: 3, SEQ ID NO: 11, SEQ ID NO: 12 |
| | SEQ ID NO: 3, SEQ ID NO: 11, SEQ ID NO: 13 |
| | SEQ ID NO: 3, SEQ ID NO: 11, SEQ ID NO: 28 |
| | SEQ ID NO: 3, SEQ ID NO: 12, SEQ ID NO: 13 |
| | SEQ ID NO: 3, SEQ ID NO: 12, SEQ ID NO: 28 |
| | SEQ ID NO: 3, SEQ ID NO: 13, SEQ ID NO: 28 |
| | SEQ ID NO: 8, SEQ ID NO: 11, SEQ ID NO: 12 |
| | SEQ ID NO: 8, SEQ ID NO: 11, SEQ ID NO: 13 |
| | SEQ ID NO: 8, SEQ ID NO: 11, SEQ ID NO: 28 |
| | SEQ ID NO: 8, SEQ ID NO: 12, SEQ ID NO: 13 |
| | SEQ ID NO: 8, SEQ ID NO: 12, SEQ ID NO: 28 |
| | SEQ ID NO: 8, SEQ ID NO: 13, SEQ ID NO: 28 |
| | SEQ ID NO: 11, SEQ ID NO: 12, SEQ ID NO: 13 |
| | SEQ ID NO: 11, SEQ ID NO: 12, SEQ ID NO: 28 |
| | SEQ ID NO: 11, SEQ ID NO: 13, SEQ ID NO: 28 |
| | SEQ ID NO: 12, SEQ ID NO: 13, SEQ ID NO: 28 |
| 4 | SEQ ID NO: 1, SEQ ID NO: 3, SEQ ID NO: 8, |
| | SEQ ID NO: 11 |
| | SEQ ID NO: 1, SEQ ID NO: 3, SEQ ID NO: 8, |
| | SEQ ID NO: 12 |
| | SEQ ID NO: 1, SEQ ID NO: 3, SEQ ID NO: 8, |
| | SEQ ID NO: 13 |
| | SEQ ID NO: 1, SEQ ID NO: 3, SEQ ID NO: 8, |
| | SEQ ID NO: 28 |
| | SEQ ID NO: 1, SEQ ID NO: 3, SEQ ID NO: 11, |
| | SEQ ID NO: 12 |
| | SEQ ID NO: 1, SEQ ID NO: 3, SEQ ID NO: 11, |
| | SEQ ID NO: 13 |
| | SEQ ID NO: 1, SEQ ID NO: 3, SEQ ID NO: 11, |
| | SEQ ID NO: 28 |
| | SEQ ID NO: 1, SEQ ID NO: 3, SEQ ID NO: 12, |
| | SEQ ID NO: 13 |
| | SEQ ID NO: 1, SEQ ID NO: 3, SEQ ID NO: 12, |
| | SEQ ID NO: 28 |
| | SEQ ID NO: 1, SEQ ID NO: 3, SEQ ID NO: 13, |
| | SEQ ID NO: 28 |
| | SEQ ID NO: 1, SEQ ID NO: 8, SEQ ID NO: 11, |
| | SEQ ID NO: 12 |
| | SEQ ID NO: 1, SEQ ID NO: 8, SEQ ID NO: 11, |
| | SEQ ID NO: 13 |
| | SEQ ID NO: 1, SEQ ID NO: 8, SEQ ID NO: 11, |
| | SEQ ID NO: 28 |
| | SEQ ID NO: 1, SEQ ID NO: 8, SEQ ID NO: 12, |
| | SEQ ID NO: 13 |
| | SEQ ID NO: 1, SEQ ID NO: 8, SEQ ID NO: 12, |
| | SEQ ID NO: 28 |
| | SEQ ID NO: 1, SEQ ID NO: 8, SEQ ID NO: 13, |
| | SEQ ID NO: 28 |
| | SEQ ID NO: 1, SEQ ID NO: 11, |
| | SEQ ID NO: 12, SEQ ID NO: 13 |
| | SEQ ID NO: 1, SEQ ID NO: 11, |
| | SEQ ID NO: 12, SEQ ID NO: 28 |
| | SEQ ID NO: 1, SEQ ID NO: 11, |
| | SEQ ID NO: 13, SEQ ID NO: 28 |
| | SEQ ID NO: 1, SEQ ID NO: 12, |
| | SEQ ID NO: 13, SEQ ID NO: 28 |
| | SEQ ID NO: 3, SEQ ID NO: 8, SEQ ID NO: 11, |
| | SEQ ID NO: 12 |
| | SEQ ID NO: 3, SEQ ID NO: 8, SEQ ID NO: 11, |
| | SEQ ID NO: 13 |
| | SEQ ID NO: 3, SEQ ID NO: 8, SEQ ID NO: 11, |
| | SEQ ID NO: 28 |
| | SEQ ID NO: 3, SEQ ID NO: 8, SEQ ID NO: 12, |
| | SEQ ID NO: 13 |
| | SEQ ID NO: 3, SEQ ID NO: 8, SEQ ID NO: 12, |
| | SEQ ID NO: 28 |
| | SEQ ID NO: 3, SEQ ID NO: 8, SEQ ID NO: 13, |
| | SEQ ID NO: 28 |
| | SEQ ID NO: 3, SEQ ID NO: 11, |
| | SEQ ID NO: 12, SEQ ID NO: 13 |
| | SEQ ID NO: 3, SEQ ID NO: 11, |
| | SEQ ID NO: 12, SEQ ID NO: 28 |
| | SEQ ID NO: 3, SEQ ID NO: 11, |
| | SEQ ID NO: 13, SEQ ID NO: 28 |
| | SEQ ID NO: 3, SEQ ID NO: 12, |
| | SEQ ID NO: 13, SEQ ID NO: 28 |
| | SEQ ID NO: 8, SEQ ID NO: 11, |
| | SEQ ID NO: 12, SEQ ID NO: 13 |
| | SEQ ID NO: 8, SEQ ID NO: 11, |
| | SEQ ID NO: 12, SEQ ID NO: 28 |
| | SEQ ID NO: 8, SEQ ID NO: 11, |
| | SEQ ID NO: 13, SEQ ID NO: 28 |
| | SEQ ID NO: 8, SEQ ID NO: 12, |
| | SEQ ID NO: 13, SEQ ID NO: 28 |
| | SEQ ID NO: 11, SEQ ID NO: 12, |
| | SEQ ID NO: 13, SEQ ID NO: 28 |
| 5 | SEQ ID NO: 1, SEQ ID NO: 3, SEQ ID NO: 8, |
| | SEQ ID NO: 11, SEQ ID NO: 12 |
| | SEQ ID NO: 1, SEQ ID NO: 3, SEQ ID NO: 8, |
| | SEQ ID NO: 11, SEQ ID NO: 13 |
| | SEQ ID NO: 1, SEQ ID NO: 3, SEQ ID NO: 8, |
| | SEQ ID NO: 11, SEQ ID NO: 28 |
| | SEQ ID NO: 1, SEQ ID NO: 3, SEQ ID NO: 8, |
| | SEQ ID NO: 12, SEQ ID NO: 13 |
| | SEQ ID NO: 1, SEQ ID NO: 3, SEQ ID NO: 8, |
| | SEQ ID NO: 12, SEQ ID NO: 28 |
| | SEQ ID NO: 1, SEQ ID NO: 3, SEQ ID NO: 8, |
| | SEQ ID NO: 13, SEQ ID NO: 28 |
| | SEQ ID NO: 1, SEQ ID NO: 3, SEQ ID NO: 11, |
| | SEQ ID NO: 12, SEQ ID NO: 13 |
| | SEQ ID NO: 1, SEQ ID NO: 3, SEQ ID NO: 11, |
| | SEQ ID NO: 12, SEQ ID NO: 28 |
| | SEQ ID NO: 1, SEQ ID NO: 3, SEQ ID NO: 11, |
| | SEQ ID NO: 13, SEQ ID NO: 28 |
| | SEQ ID NO: 1, SEQ ID NO: 3, SEQ ID NO: 12, |
| | SEQ ID NO: 13, SEQ ID NO: 28 |
| | SEQ ID NO: 1, SEQ ID NO: 8, SEQ ID NO: 11, |
| | SEQ ID NO: 12, SEQ ID NO: 13 |
| | SEQ ID NO: 1, SEQ ID NO: 8, SEQ ID NO: 11, |
| | SEQ ID NO: 12, SEQ ID NO: 28 |
| | SEQ ID NO: 1, SEQ ID NO: 8, SEQ ID NO: 11, |
| | SEQ ID NO: 13, SEQ ID NO: 28 |
| | SEQ ID NO: 1, SEQ ID NO: 8, SEQ ID NO: 12, |
| | SEQ ID NO: 13, SEQ ID NO: 28 |
| | SEQ ID NO: 1, SEQ ID NO: 11, SEQ ID NO: 12, |
| | SEQ ID NO: 13, SEQ ID NO: 28 |
| | SEQ ID NO: 3, SEQ ID NO: 8, SEQ ID NO: 11, |
| | SEQ ID NO: 12, SEQ ID NO: 13 |
| | SEQ ID NO: 3, SEQ ID NO: 8, SEQ ID NO: 11, |
| | SEQ ID NO: 12, SEQ ID NO: 28 |
| | SEQ ID NO: 3, SEQ ID NO: 8, SEQ ID NO: 11, |
| | SEQ ID NO: 13, SEQ ID NO: 28 |
| | SEQ ID NO: 3, SEQ ID NO: 8, SEQ ID NO: 12, |
| | SEQ ID NO: 13, SEQ ID NO: 28 |
| | SEQ ID NO: 3, SEQ ID NO: 11, SEQ ID NO: 12, |
| | SEQ ID NO: 13, SEQ ID NO: 28 |
| | SEQ ID NO: 8, SEQ ID NO: 11, SEQ ID NO: 12, |
| | SEQ ID NO: 13, SEQ ID NO: 28 |
| 6 | SEQ ID NO: 1, SEQ ID NO: 3, SEQ ID NO: 8, |
| | SEQ ID NO: 11, SEQ ID NO: 12, SEQ ID NO: 13 |
| | SEQ ID NO: 1, SEQ ID NO: 3, SEQ ID NO: 8, |
| | SEQ ID NO: 11, SEQ ID NO: 12, SEQ ID NO: 28 |
| | SEQ ID NO: 1, SEQ ID NO: 3, SEQ ID NO: 8, |
| | SEQ ID NO: 11, SEQ ID NO: 13, SEQ ID NO: 28 |
| | SEQ ID NO: 1, SEQ ID NO: 3, SEQ ID NO: 8, |
| | SEQ ID NO: 12, SEQ ID NO: 13, SEQ ID NO: 28 |
| | SEQ ID NO: 1, SEQ ID NO: 3, SEQ ID NO: 11, |
| | SEQ ID NO: 12, SEQ ID NO: 13, SEQ ID NO: 28 |
| | SEQ ID NO: 1, SEQ ID NO: 8, SEQ ID NO: 11, |
| | SEQ ID NO: 12, SEQ ID NO: 13, SEQ ID NO: 28 |
| | SEQ ID NO: 3, SEQ ID NO: 8, SEQ ID NO: 11, |
| | SEQ ID NO: 12, SEQ ID NO: 13, SEQ ID NO: 28 |
| 7 | SEQ ID NO: 1, SEQ ID NO: 3, SEQ ID NO: 8, |
| | SEQ ID NO: 11, SEQ ID NO: 12, |
| | SEQ ID NO: 13, SEQ ID NO: 28 |

The method for determining in vitro or ex vivo the immune status of an individual, as described above, according to any embodiment, can also comprise a step of detecting and/or quantifying the expression, in the test biological sample, of at least one, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95 gene(s) selected from the following genes:
CD74, CX3CR1, IL-10, S100A8, S100A9, MERTK, CLEC7A, CD36, TIMP2, CCL13, PTGS2, IL-12B, IL-6, IL-1A, CCL20, MX1, OAS-1, CCL15, OAS-3, EIF2AK2, IFNγ, NEFH, MMP10, SERPINB2, THBD, STAT1, CCR4, HLA-DRB1/B3, TCF7, EOMES, BCL11B, ITGA7, IL-18R1, NLRC4, CYP1B1, HGF, IL-5RA, CCLP4, CD3G, CD4OLG, CD3D, CD127, ICOS, IL-1R2, IL-1RN, IL-18, IL-18RAP, OX40L, PD-1, PD-L1, Zonulin (HP), BTLA, C3AR1, CD154, GM-CSF, IFIH1, IL-15, MCP1, PCSK9, STAT4, LTR82B, CIITA, LILRB2, CD177, ADGRE3, FLT-1, CD64, TREM-1, TNF-α, IL-1β, ALOX5, IL-17A, NFκB, TBX21, HIF1a, RORgT, OAS-2, GNLY, CTLA-4, TIM 3, CD274, IL-2, IL-7R, GATA3, CXCL10, FAS, GSN, MDC1, DYRK2, TDRD9, CNB1IP1, ZAP70 and ARL14EP.

Preferably, the invention relates to a method for determining the immune status of an individual, as described above, in which the expression is detected and/or quantified at the RNA transcript level or messenger RNA (mRNA). The detection and/or quantification at the RNA transcript or mRNA level can be carried out by any means known to one skilled in the art. Particularly, it can be cited as examples:
hybridization methods, preferably with a hybridization chip, by in situ hybridization or by Northern blot;
amplification methods, preferably by RT-PCR («Reverse Transcriptase Polymerase Chain Reaction»), more preferably by RT-qPCR (quantitative RT-PCR). Mention may in particular be made of nested PCR. The PCR reactions can also be multiplexed;
sequencing methods, preferably by high throughput sequencing

Preferably, the invention relates to a method for determining the immune status of an individual, as described above, in which the immune status is determined as being an immunosuppression status (or immunodepression or immunodeficiency status or hypoactive immune status or immune paralysis), a normal immune status (or immunocompetence status), or inflammation status (or hyperactive immune status). The inflammation status includes the hyper-inflammation status.

Preferably, the invention relates to a method for determining the immune status of an individual, as described above, in which the individual is a patient admitted within a medical facility, preferably in an intensive care unit, in emergency department or in resuscitation. Also preferably, the individual is a trauma patient, a patient with burns, a surgical patient or a patient with sepsis, preferably a patient with septic shock. Even more preferably, the test biological sample is obtained by sampling made within 10 days, preferably within 9 days, preferably within 8 days, preferably within 7 days, preferably within 6 days, preferably within 5 days, preferably within 4 days, preferably within 3 days, preferably within 2 days, preferably within 24 hours, following admission to the medical facility.

Preferentially, the invention relates to a method for determining the immune status of an individual, as described above, in which the reference biological sample is a biological sample obtained from a healthy individual, preferably a biological sample obtained from the same individual from which the test biological sample was obtained but collected before infection or aggression, or a biological sample from an individual of known immune status, preferably with inflammation status, normal immune status, or an immunosuppression status.

Preferably, the test biological sample and/or the reference biological sample, as used in the method for determining the immune status of an individual, according to the invention, as described above, is a blood sample, preferably a sample of whole blood, plasma or serum, or a sample of peripheral blood mononuclear cells, extracted from a blood sample.

Preferably, the method for determining the immune status according to the invention, as described above, comprises a step of administering a treatment, preferably an immunomodulatory treatment, adapted to the immune status of the individual. Preferably, the immunomodulatory treatment is an immunostimulatory treatment, if it is determined that the individual has an immunosuppression status, or an anti-inflammatory treatment, if it is determined that the individual has an inflammatory status. Among the immunostimulant treatments which can be selected, mention may be made, by way of example, of the group of interleukins, in particular IL-7, IL-15 or IL-3, growth factors, in particular GM-CSF, interferons, in particular IFNγ, Toll agonists, antibodies, in particular anti-PD1, anti-PDL1, anti-LAG3, anti-TIM3, anti-IL-10 or anti-CTLA4 antibodies, transferrins and inhibitory molecules of apoptosis, FLT3L, Thymosin α1, adrenergic antagonists. Among the anti-inflammatory treatments, mention may in particular be made of the group of glucocorticoids, cytostatic agents, molecules acting on immunophilins and cytokines, molecules which block the IL-1 receptor and anti-TNF treatments.

The present invention also relates to the use of at least one, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34 sequence(s) selected from the sequences identified in SEQ ID NO: 1 to 34, as appearing in Lists 1 to 4, or from the sequences which exhibit at least 99% identity with one of the sequences identified in SEQ ID NO: 1 to 34, to determine in vitro or ex vivo the immune status of an individual, preferably a patient.

The present invention also relates to a method for identifying or selecting a treatment, preferably an immunomodulatory treatment, more particularly an immunostimulating treatment or an anti-inflammatory treatment, suitable for treating an individual, preferably a patient, comprising the following steps of:
a. determining the immune status of said individual by a method as previously described
b. identifying an appropriate treatment from the immune status determined in step a)

Preferably, the immunomodulatory treatment is an immunostimulatory treatment, if it is determined that the individual has an immunosuppression status, or an anti-inflammatory treatment, if it is determined that the individual has an inflammation status.

The present invention further relates to a method for evaluating the effectiveness of a treatment, preferably an immunomodulatory treatment, more particularly an immunostimulating treatment or an anti-inflammatory treatment, on an individual, preferably a patient, comprising the following steps of:
a. detecting and/or quantifying the expression of at least part of a sequence selected from the sequences identified in SEQ ID NO: 1 to 34 or from the sequences which exhibit at least 99% identity with one of the identified sequences in SEQ ID NO: 1 to 34, in a first biological sample of said individual, collected before the treatment, and in a second biological sample of said individual, collected after the treatment
b. comparing the expression obtained for the 2 biological samples in step a)
c. evaluating the effectiveness of the treatment from the comparison of step b)

Another subject of the present invention concerns an amplification primer comprising, or consisting of, a nucleotide sequence complementary to at least part of a sequence selected from the sequences identified in SEQ ID NO: 1 to 34 or from the sequences which exhibit at least 99% identity with one of the sequences identified in SEQ ID NO: 1 to 34, and the sequences complementary to these sequences. Preferably, the amplification primer according to the invention is selected from the primers presented in Table 6.

TABLE 6

| SEQ ID NO: | Primer # | Nucleotide sequence |
|---|---|---|
| 35 | 1A | TGTACAAAACTCAAATGGTCTTC |
| 36 | 1B | ATGACCAACTTAGATTTCCTTGA |
| 37 | 2A | GCCAGAGAGGCATAATGAAGCA |
| 38 | 2B | GATTCTAAGCCTCCCCCTCATTT |
| 39 | 3A | TGGCTCATAGGGATTCCAGACT |
| 40 | 3B | AGCAAGTTGTCAAGAGCCAATCT |
| 41 | 4A | CACTCTAGGAATCTTAGGCA |
| 42 | 4B | TGAAACCAATAGTCCAGTG |
| 43 | 5A | TTCTACTGTTCACTGCTATCCTCC |
| 44 | 5B | CCTGTGGCAGCTTTTTGAAGTAA |
| 45 | 6A | AGAGCAGAAGAAGATGGATACT |
| 46 | 6B | CATGAGCTGACATCATCCAAT |
| 47 | 7A | TCTGTACTGGTTGCCCCAAC |
| 48 | 7B | CGTGCCAGGCCTCTAATACTTTT |
| 49 | 8A | AGGGAAGACCCCAAGATGATG |
| 50 | 8B | CATGCAAAGTCCAACGAGAGG |
| 51 | 9A | GGGTGGCTGCATCCTATGG |
| 52 | 9B | CTGGTCAGGAAAAAATTTGCCTTC |
| 53 | 10A | ACATGACATTGTCTGAACTTTGGG |
| 54 | 10B | TAGGACCATGCAGATACTAGTGAC |
| 55 | 11A | GAACTCCACAAACCTTGA |
| 56 | 11B | GCTAGAAGCTTTGGATATCT |
| 57 | 12A | TGGCTGTTACAACTTTCATG |
| 58 | 12B | TCTCCCTATTCTGAGCACA |

The present invention also concerns a pair of amplification primers, consisting of two amplification primers selected from the primers as described above, and making it possible to amplify, preferably making it possible to specifically amplify, at least part of a sequence selected from the sequences identified in SEQ ID NO: 1 to 34 or from the sequences which exhibit at least 99% identity with one of the sequences identified in SEQ ID NO: 1 to 34, and the sequences complementary to these sequences. Preferably, the pair of amplification primers according to the invention is selected from the pairs of primers presented in Table 7.

TABLE 7

| Pair of Primers # | Primers # |
|---|---|
| 1 | Forward: Primer #1A |
| | Reverse: Primer #1B |
| 2 | Forward: Primer #2A |
| | Reverse: Primer #2B |
| 3 | Forward: Primer #3A |
| | Reverse: Primer #3B |
| 4 | Forward: Primer #4A |
| | Reverse: Primer #4B |
| 5 | Forward: Primer #5A |
| | Reverse: Primer #5B |
| 6 | Forward: Primer #6A |
| | Reverse: Primer #6B |
| 7 | Forward: Primer #7A |
| | Reverse: Primer #7B |
| 8 | Forward: Primer #8A |
| | Reverse: Primer #8B |
| 9 | Forward: Primer #9A |
| | Reverse: Primer #9B |
| 10 | Forward: Primer #10A |
| | Reverse: Primer #10B |
| 11 | Forward: Primer #11A |
| | Reverse: Primer #11B |
| 12 | Forward: Primer #12A |
| | Reverse: Primer #12B |

Another subject of the present invention concerns a hybridization probe, whose nucleotide sequence comprises, or consists of, a nucleotide sequence complementary to at least part of a sequence selected from the sequences identified in SEQ ID NO: 1 to 34 or from the sequences which have at least 99% identity with one of the sequences identified in SEQ ID NO: 1 to 34, and the sequences complementary to these sequences. Preferably, the hybridization probe according to the invention is selected from the hybridization probes presented in Table 8.

TABLE 8

| SEQ | | | |
|---|---|---|---|
| ID | Probe | | |
| NO: | # | HERV-V3 chip probe name | Nucleotide sequence |
| 59 | 1A | 190665001-HERV0376uL_at1 | ATGACCAACTTAGATTTCCTTGAGT |
| 60 | 1B | 190665001-HERV0376uL_at2 | GTCAAGGGTAAAGCTGTGAAAGTTT |
| 61 | 1C | 190665001-HERV0376uL_at3 | GGAAGACCATTTGAGTTTTGTACAC |
| 62 | 1A′ | 190665001-HERV0376uL_st1 | ACTCAAGGAAATCTAAGTTGGTCAT |
| 63 | 1B′ | 190665001-HERV0376uL_st2 | AAACTTTCACAGCTTTACCCTTGAC |
| 64 | 1C′ | 190665001-HERV0376uL_st3 | GTGTACAAAACTCAAATGGTCTTCC |
| 65 | 1D | 190665002-HERV0376uL_at1 | GAAGATATGGGCCAGAACTTGTATA |
| 66 | 1E | 190665002-HERV0376uL_at2 | CAGGACCTGAGTTAAGCCAAGAATA |
| 67 | 1F | 190665002-HERV0376uL_at3 | ACCTGAGTTAAGCCAAGAATACAGT |
| 68 | 1D′ | 190665002-HERV0376uL_st1 | TATACAAGTTCTGGCCCATATCTTC |
| 69 | 1E′ | 190665002-HERV0376uL_st2 | TATTCTTGGCTTAACTCAGGTCCTG |
| 70 | 1F′ | 190665002-HERV0376uL_st3 | ACTGTATTCTTGGCTTAACTCAGGT |
| 71 | 2A | 220247002-HERV0797uL_at1 | GTAAGATTCTAAGCCTCCCCCTCAT |
| 72 | 2B | 220247002-HERV0797uL_at2 | GATTCTAAGCCTCCCCCTCATTTAA |
| 73 | 2C | 220247002-HERV0797uL_at3 | CTAAGCCTCCCCCTCATTTAAAGGA |
| 74 | 2A′ | 220247002-HERV0797uL_st1 | ATGAGGGGGAGGCTTAGAATCTTAC |
| 75 | 2B′ | 220247002-HERV0797uL_st2 | TTAAATGAGGGGGAGGCTTAGAATC |
| 76 | 2C′ | 220247002-HERV0797uL_st3 | TCCTTTAAATGAGGGGGAGGCTTAG |
| 77 | 3A | 170369402HE41env_at1 | ATGGCTCATAGGGATTCCAGACTCC |
| 78 | 3B | 170369402HE41env_at2 | GGCTCATAGGGATTCCAGACTCCCA |
| 79 | 3C | 170369402HE41env_at3 | CTCATAGGGATTCCAGACTCCCATT |
| 80 | 3A′ | 170369402HE41env_st1 | GGAGTCTGGAATCCCTATGAGCCAT |
| 81 | 3B′ | 170369402HE41env_st2 | TGGGAGTCTGGAATCCCTATGAGCC |
| 82 | 3C′ | 170369402HE41env_st3 | AATGGGAGTCTGGAATCCCTATGAG |
| 83 | 4A | 121601801-HERV0492uL_at1 | TGAAACCAATAGTCCAGTGGTGGCC |
| 84 | 4B | 121601801-HERV0492uL_at2 | TTCCAGTGATTTAGATAAAATCCCT |
| 85 | 4C | 121601801-HERV0492uL_at3 | TTTCTGCCTAAGATTCCTAGAGTGC |
| 86 | 4A′ | 121601801-HERV0492uL_st1 | GGCCACCACTGGACTATTGGTTTCA |
| 87 | 4B′ | 121601801-HERV0492uL_st2 | AGGGATTTTATCTAAATCACTGGAA |
| 88 | 4C′ | 121601801-HERV0492uL_st3 | GCACTCTAGGAATCTTAGGCAGAAA |
| 89 | 5A | 011052702-MALR1044uL_at1 | CTGTGGCAGCTTTTTGAAGTAAGGA |
| 90 | 5B | 011052702-MALR1044uL_at2 | ATGGTTAGTGCAGAGTAAAGTTTGG |
| 91 | 50 | 011052702-MALR1044uL_at3 | AGGATAGCAGTGAACAGTAGAATGG |
| 92 | 5A′ | 011052702-MALR1044uL_st1 | TCCTTACTTCAAAAAGCTGCCACAG |
| 93 | 5B′ | 011052702-MALR1044uL_st2 | CCAAACTTTACTCTGCACTAACCAT |
| 94 | 5C′ | 011052702-MALR1044uL_st3 | CCATTCTACTGTTCACTGCTATCCT |
| 95 | 6A | 011052202-HERV1033uL_at1 | AGATCCAACATGAGCTGACATCATC |
| 96 | 6A′ | 011052202-HERV1033uL_st1 | GATGATGTCAGCTCATGTTGGATCT |
| 97 | 7A | 130360601-HERV0808cL at1 | GAGGTTGGGGCAACCAGTACAGATT |
| 98 | 7A′ | 130360601-HERV0808cL_st1 | AATCTGTACTGGTTGCCCCAACCTC |
| 99 | 8A | 141107102-MALR1019uL_at1 | CCCCAAGATGATGGACTCTGGTGAT |
| 100 | 8B | 141107102-MALR1019uL_at2 | CACTGCCATCACTTTGGGAAAGACT |
| 101 | 8C | 141107102-MALR1019uL_at3 | AAGCAGCCTCTCGTTGGACTTTGCA |
| 102 | 8A′ | 141107102-MALR1019uL_st1 | ATCACCAGAGTCCATCATCTTGGGG |
| 103 | 8B′ | 141107102-MALR1019uL_st2 | AGTCTTTCCCAAAGTGATGGCAGTG |
| 104 | 8C′ | 141107102-MALR1019uL_st3 | TGCAAAGTCCAACGAGAGGCTGCTT |
| 105 | 9A | 021460102-HERV0599uL_at1 | GAGGGCAGTTTGGAACAGTTGGAAC |
| 106 | 9B | 021460102-HERV0599uL_at2 | TGAGAGACGATTATCTGGAAGAAGA |
| 107 | 9C | 021460102-HERV0599uL_at3 | TCACAGCTTGAGAATGTGGTAGGAG |
| 108 | 9D | 021460102-HERV0599uL_at4 | GGAATGGGGGGCATGGAATTAAAGC |
| 109 | 9A′ | 021460102-HERV0599uL_st1 | GTTCCAACTGTTCCAAACTGCCCTC |
| 110 | 9B′ | 021460102-HERV0599uL_st2 | TCTTCTTCCAGATAATCGTCTCTCA |
| 111 | 9C′ | 021460102-HERV0599uL_st3 | CTCCTACCACATTCTCAAGCTGTGA |
| 112 | 9D′ | 021460102-HERV0599uL_st4 | GCTTTAATTCCATGCCCCCCATTCC |
| 113 | 10A | 021456001-MALR1017uL_at1 | AGTCCCTAACTGTCTGCAAACCCAC |
| 114 | 10B | 021456001-MALR1017uL_at2 | ACTGTCTGCAAACCCACAATGGACC |
| 115 | 10C | 021456001-MALR1017uL_at3 | CAATGGACCTGTTGCATGTGTAAGA |
| 116 | 10A′ | 021456001-MALR1017uL_st1 | GTGGGTTTGCAGACAGTTAGGGACT |
| 117 | 10B′ | 021456001-MALR1017uL_st2 | GGTCCATTGTGGGTTTGCAGACAGT |
| 118 | 10C′ | 021456001-MALR1017uL_st3 | TCTTACACATGCAACAGGTCCATTG |
| 119 | 11A | 050286701-HERV0513uL_at1 | CTGCATCCTATGGTGTTTCTACATG |
| 120 | 11B | 050286701-HERV0513uL_at2 | ATAATCTTTTCCGGCATGTTGGTAT |
| 121 | 11C | 050286701-HERV0513uL_at3 | TAAAGATAGTGTTTCCTATTGTGTC |
| 122 | 11A′ | 050286701-HERV0513uL_st1 | CATGTAGAAACACCATAGGATGCAG |
| 123 | 11B′ | 050286701-HERV0513uL_st2 | ATACCAACATGCCGGAAAAGATTAT |
| 124 | 11C′ | 050286701-HERV0513uL_st3 | GACACAATAGGAAACACTATCTTTA |
| 125 | 12A | 050287402-MALR1022uL_at1 | ACAGAGACTGCAAGAGTAATGACAT |
| 126 | 12B | 050287402-MALR1022uL_at2 | TCTGAACTTTGGGAAACAATTATGT |
| 127 | 12C | 050287402-MALR1022uL_at3 | ACTTTCCAGTTAATCGAATCAATCC |
| 128 | 12D | 050287402-MALR1022uL_at4 | TTTTAACCTAGACTAGTTCCAACTG |
| 129 | 12E | 050287402-MALR1022uL_at5 | GTCACTAGTATCTGCATGGTCCTAA |
| 130 | 12A′ | 050287402-MALR1022uL_st1 | ATGTCATTACTCTTGCAGTCTCTGT |
| 131 | 12B′ | 050287402-MALR1022uL_st2 | ACATAATTGTTTCCCAAAGTTCAGA |
| 132 | 12C′ | 050287402-MALR1022uL_st3 | GGATTGATTCGATTAACTGGAAAGT |
| 133 | 12D′ | 050287402-MALR1022uL_st4 | CAGTTGGAACTAGTCTAGGTTAAAA |
| 134 | 12E′ | 050287402-MALR1022uL_st5 | TTAGGACCATGCAGATACTAGTGAC |
| 135 | 13A | 052182701-MALR1129uL_at1 | TTATTCCAGTCACCTCGAGTCATTC |
| 136 | 13B | 052182701-MALR1129uL_at2 | TCATCCTAGCCGTCGTAGAGCAGAG |
| 137 | 13C | 052182701-MALR1129uL_at3 | TGCCCTTCTGACTCCTTGACAGTGG |
| 138 | 13A′ | 052182701-MALR1129uL_st1 | GAATGACTCGAGGTGACTGGAATAA |
| 139 | 13B′ | 052182701-MALR1129uL_st2 | CTCTGCTCTACGACGGCTAGGATGA |
| 140 | 13C′ | 052182701-MALR1129uL_st3 | CCACTGTCAAGGAGTCAGAAGGGCA |
| 141 | 14A | 190478501-MALR1003cL_at1 | TAAGTGGGACCAAGACACAAACCAA |
| 142 | 14B | 190478501-MALR1003cL_at3 | ACCAAGACACAAACCAACATGCCTG |
| 143 | 14A′ | 190478501-MALR1003cL_st1 | TTGGTTTGTGTCTTGGTCCCACTTA |
| 144 | 14B′ | 190478501-MALR1003cL_st3 | CAGGCATGTTGGTTTGTGTCTTGGT |
| 145 | 15A | 011790601ERV9sLU5p_at1 | CTGAGGTCCATGGCTTCTTTCCTTG |
| 146 | 15A′ | 011790601ERV9sLU5p_st1 | CAAGGAAAGAAGCCATGGACCTCAG |
| 147 | 16A | 052681601-MALR1018uL_at1 | CCTTTGTTTTCCTACTGACAGGTCC |
| 148 | 16B | 052681601-MALR1018uL_at2 | TTCAAAATATTTAACTCTCCAGGCT |
| 149 | 16C | 052681601-MALR1018uL_at3 | GAGGTCACATGACTCTGTTGTGGAC |
| 150 | 16A′ | 052681601-MALR1018uL_st1 | GGACCTGTCAGTAGGAAAACAAAGG |
| 151 | 16B′ | 052681601-MALR1018uL_st2 | AGCCTGGAGAGTTAAATATTTTGAA |
| 152 | 16C′ | 052681601-MALR1018uL_st3 | GTCCACAACAGAGTCATGTGACCTC |
| 153 | 17A | 160627301-MALR1014uL_at1 | CAGCTGAGATCCGTTGACGCCAGCC |
| 154 | 17B | 160627301-MALR1014uL_at2 | TCCGACATGTGGGTGAACTCAGCCA |
| 155 | 17C | 160627301-MALR1014uL_at3 | TTCTCAGCCATGTGTTTTGTGAACT |
| 156 | 17A′ | 160627301-MALR1014uL_st1 | GGCTGGCGTCAACGGATCTCAGCTG |
| 157 | 17B′ | 160627301-MALR1014uL_st2 | TGGCTGAGTTCACCCACATGTCGGA |
| 158 | 17C′ | 160627301-MALR1014uL_st3 | AGTTCACAAAACACATGGCTGAGAA |
| 159 | 18A | 111686702-HERV0861uL_at1 | TTGAGGCAGGACAGAACCAGGCTCC |
| 160 | 18B | 111686702-HERV0861uL_at2 | GGACAGAACCAGGCTCCTGTTAGTC |
| 161 | 18C | 111686702-HERV0861uL_at3 | AGTTTACTGAGCAGTGACTTTGTGT |
| 162 | 18A′ | 111686702-HERV0861uL_st1 | GGAGCCTGGTTCTGTCCTGCCTCAA |
| 163 | 18B′ | 111686702-HERV0861uL_st2 | GACTAACAGGAGCCTGGTTCTGTCC |
| 164 | 18C′ | 111686702-HERV0861uL_st3 | ACACAAAGTCACTGCTCAGTAAACT |
| 165 | 19A | 040318302-MALR1134uL_at1 | ATAGGGATGATCCTGCACGAATGGC |
| 166 | 19B | 040318302-MALR1134uL_at2 | GGATGATCCTGCACGAATGGCATGG |
| 167 | 19A′ | 040318302-MALR1134uL_st1 | GCCATTCGTGCAGGATCATCCCTAT |
| 168 | 19B′ | 040318302-MALR1134uL_st2 | CCATGCCATTCGTGCAGGATCATCC |
| 169 | 20A | 041529101-MALR1026uL_at1 | AGTGGACACTTTTTAGGATGTCTGC |
| 170 | 20B | 041529101-MALR1026uL_at2 | GCCCTGACATAAGAGTTTGCCAGTT |
| 171 | 20C | 041529101-MALR1026uL_at3 | CCTGTACCCACCTTTCACCAGAGCT |
| 172 | 20A′ | 041529101-MALR1026uL_st1 | GCAGACATCCTAAAAAGTGTCCACT |
| 173 | 20B′ | 041529101-MALR1026uL_st2 | AACTGGCAAACTCTTATGTCAGGGC |
| 174 | 20C′ | 041529101-MALR1026uL_st3 | AGCTCTGGTGAAAGGTGGGTACAGG |
| 175 | 21A | 141106902-MALR1133uL_at1 | AATTGTTGGAATTTGAAAGTGGGGT |
| 176 | 21A′ | 141106902-MALR1133uL_st1 | ACCCCACTTTCAAATTCCAACAATT |
| 177 | 22A | 060281701-MALR1043uL_at1 | GTCAGCACCGTGCTTCTCTAACTTT |
| 178 | 22B | 060281701-MALR1043uL_at2 | GCACCGTGCTTCTCTAACTTTCCAC |
| 179 | 22C | 060281701-MALR1043uL_at3 | CGTGCTTCTCTAACTTTCCACCTGC |
| 180 | 22A′ | 060281701-MALR1043uL_st1 | AAAGTTAGAGAAGCACGGTGCTGAC |
| 181 | 22B′ | 060281701-MALR1043uL_st2 | GTGGAAAGTTAGAGAAGCACGGTGC |
| 182 | 22C′ | 060281701-MALR1043uL_st3 | GCAGGTGGAAAGTTAGAGAAGCACG |
| 183 | 23A | 043166601-MALR1018uL_at1 | CAGCCTCGCACCTAAGAACGCCGTG |
| 184 | 23B | 043166601-MALR1018uL_at2 | CAGTGAGAAATCTGCTGGGGATGCC |
| 185 | 23C | 043166601-MALR1018uL_at3 | GAAAGGGACATACCTGGCAGGTGCC |
| 186 | 23A′ | 043166601-MALR1018uL_st1 | CACGGCGTTCTTAGGTGCGAGGCTG |
| 187 | 23B′ | 043166601-MALR1018uL_st2 | GGCATCCCCAGCAGATTTCTCACTG |
| 188 | 23C′ | 043166601-MALR1018uL_st3 | GGCACCTGCCAGGTATGTCCCTTTC |
| 189 | 24A | 100090601-HERV0429uL_at1 | GGTAGAGACCGAGGCGGATATACAG |
| 190 | 24B | 100090601-HERV0429uL_at3 | GAGACCGAGGCGGATATACAGGCCT |
| 191 | 24A′ | 100090601-HERV0429uL_st1 | CTGTATATCCGCCTCGGTCTCTACC |
| 192 | 24B′ | 100090601-HERV0429uL_st3 | AGGCCTGTATATCCGCCTCGGTCTC |
| 193 | 25A | 061529601-HERV0492uL_at1 | TATACTGGGGCCCAATTCTACAGAC |
| 194 | 25B | 061529601-HERV0492uL_at2 | CAGACATTACTTCTTTGCCAGTTGG |
| 195 | 25C | 061529601-HERV0492uL_at3 | GACACATTGCAAGTCTGGAAGAGGA |
| 196 | 25A′ | 061529601-HERV0492uL_st1 | GTCTGTAGAATTGGGCCCCAGTATA |
| 197 | 25B′ | 061529601-HERV0492uL_st2 | CCAACTGGCAAAGAAGTAATGTCTG |
| 198 | 25C′ | 061529601-HERV0492uL_st3 | TCCTCTTCCAGACTTGCAATGTGTC |
| 199 | 26A | 100871501-MALR1020cL_at1 | CATGATCCTGGGTGAAGCCATGTGT |
| 200 | 26B | 100871501-MALR1020cL_at2 | TGTGTCTGAGGATGAAAGGGGATGC |
| 201 | 26C | 100871501-MALR1020cL_at3 | CAGATTGATGTGACATGTGGCACCT |
| 202 | 26A′ | 100871501-MALR1020cL_st1 | ACACATGGCTTCACCCAGGATCATG |
| 203 | 26B′ | 100871501-MALR1020cL_st2 | GCATCCCCTTTCATCCTCAGACACA |
| 204 | 26C′ | 100871501-MALR1020cL_st3 | AGGTGCCACATGTCACATCAATCTG |
| 205 | 27A | 170842002-MALR1003uL_at1 | AGAGGGAGCACGGTCCCAGTACACC |
| 206 | 27B | 170842002-MALR1003uL_at2 | CACGGTCCCAGTACACCTTGAGTGT |
| 207 | 27C | 170842002-MALR1003uL_at3 | TGTTACGGCTGTCCCAGGAAAGGAA |
| 208 | 27A′ | 170842002-MALR1003uL_st1 | GGTGTACTGGGACCGTGCTCCCTCT |
| 209 | 27B′ | 170842002-MALR1003uL_st2 | ACACTCAAGGTGTACTGGGACCGTG |
| 210 | 27C′ | 170842002-MALR1003uL_st3 | TTCCTTTCCTGGGACAGCCGTAACA |
| 211 | 28A | 081921103-HERV0958ul_at1 | ACTAAGAGCAACAGCCTGAGGCTAA |
| 212 | 28B | 081921103-HERV0958ul_at2 | GGCTCACCGGAAACAGGCTGAATGT |
| 213 | 28C | 081921103-HERV0958ul_at3 | GAGACACCAGATGACCGCTTGGTCT |
| 214 | 28D | 081921103-HERV0958ul_at4 | CAGCTTCCCTAGAATTATACACCAG |
| 215 | 28E | 081921103-HERV0958ul_at5 | TACTGAAGAGGTTACTTCAACTTGC |
| 216 | 28F | 081921103-HERV0958ul_at6 | TTGTAAAAATATAAACGTGAGGCAA |
| 217 | 28A′ | 081921103-HERV0958ul_st1 | TTAGCCTCAGGCTGTTGCTCTTÅGT |
| 218 | 28B′ | 081921103-HERV0958ul_st2 | ACATTCAGCCTGTTTCCGGTGAGCC |
| 219 | 28C′ | 081921103-HERV0958ul_st3 | AGACCAAGCGGTCATCTGGTGTCTC |
| 220 | 28D′ | 081921103-HERV0958ul_st4 | CTGGTGTATAATTCTAGGGAAGCTG |
| 221 | 28E′ | 081921103-HERV0958ul_st5 | GCAAGTTGAAGTAACCTGTTCAGTA |
| 222 | 28F′ | 081921103-HERV0958ul_st6 | TTGCCTCACGTTTATATTTTTACAA |
| 223 | 28G | 081921101-HERV0958ul_at1 | GATGACAGTTAAGACCCTAGGTTGC |
| 224 | 28H | 081921101-HERV0958ul_at2 | CAATCTCAAGTCTGATGACTTGTTA |
| 225 | 28I | 081921101-HERV0958ul_at3 | AGACCCATCATTGCTAGCAGACTAT |
| 226 | 28J | 081921101-HERV0958ul_at4 | AAGGATGGGAAATGCTCAGGTCACG |
| 227 | 28K | 081921101-HERV0958ul_at5 | AGGGCTCATCCACTAACCCCCTGAA |
| 228 | 28L | 081921101-HERV0958ul_at6 | GAAATGGATACCCTTGGGTTCAACT |
| 229 | 28G′ | 081921101-HERV0958ul_st1 | GCAACCTAGGGTCTTAACTGTCATC |
| 230 | 28H′ | 081921101-HERV0958ul_st2 | TAACAAGTCATCAGACTTGAGATTG |
| 231 | 28I′ | 081921101-HERV0958ul_st3 | ATAGTCTGCTAGCAATGATGGGTCT |
| 232 | 28J′ | 081921101-HERV0958ul_st4 | CGTGACCTGAGCATTTCCCATCCTT |
| 233 | 28K′ | 081921101-HERV0958ul_st5 | TTCAGGGGGTTAGTGGATGAGCCCT |
| 234 | 28L′ | 081921101-HERV0958ul_st6 | AGTTGAACCCAAGGGTATCCATTTC |
| 235 | 28M | 081921102-HERV0958ul_at1 | GTGCCATAACGACAATTAAATTTTT |
| 236 | 28N | 081921102-HERV0958ul_at2 | AGTCTTTTGTTATCTATGGAGGACT |
| 237 | 28O | 081921102-HERV0958ul_at3 | GTTGTGTTAAAGTTTCTAATTACG |
| 238 | 28P | 081921102-HERV0958ul_at4 | GTAACTTTGGGACCAAAACAATGAA |
| 239 | 28Q | 081921102-HERV0958ul_at5 | TCATAAGCCTACTAATCCGGGATCA |
| 240 | 28R | 081921102-HERV0958ul_at6 | GGGACAAGAACTAATTCCACAGGAG |
| 241 | 28M′ | 081921102-HERV0958ul_st1 | AAAAATTTAATTGTCGTTATGGCAC |
| 242 | 28N′ | 081921102-HERV0958ul_st2 | AGTCCTCCATAGATAACAAAAGACT |
| 243 | 28O′ | 081921102-HERV0958ul_st3 | ACGTAATTAGAAACTTTAACACAAC |
| 244 | 28P′ | 081921102-HERV0958ul_st4 | TTCATTGTTTTGGTCCCAAAGTTAC |
| 245 | 28Q′ | 081921102-HERV0958ul_st5 | TGATCCCGGATTAGTAGGCTTATGA |
| 246 | 28R′ | 081921102-HERV0958ul_st6 | CTCCTGTGGAATTAGTTCTTGTCCC |
| 247 | 29A | 032622601MR41sLU5p_at1 | GCCCTTTCTTGAGGTCTGGGTCTGC |
| 248 | 29A′ | 032622601MR41sLU5p_st1 | GCAGACCCAGACCTCAAGAAAGGGC |
| 249 | 30A | 220246901-HERV0889uL_at1 | AGGCTGTAACCCCCCTTAAACTGCC |
| 250 | 30B | 220246901-HERV0889uL_at2 | CAACTATGGGGAACTTAACTGGAGT |
| 251 | 30C | 220246901-HERV0889uL_at3 | GGAGTCGTTTCAGATGGGTGCTTAC |
| 252 | 30A′ | 220246901-HERV0889uL_st1 | GGCAGTTTAAGGGGGGTTACAGCC |
| 253 | 30B′ | 220246901-HERV0889uL_st2 | ACTCCAGTTAAGTTCCCCATAGTTG |
| 254 | 30C′ | 220246901-HERV0889uL_st3 | GTAAGCACCCATCTGAAACGACTCC |
| 255 | 31A | 061827101-HERV0856uL_at1 | GAAGAGTTTCAGCCCTTCAGACAAC |
| 256 | 31B | 061827101-HERV0856uL_at2 | GATCCAGGTTTGTCACGCAAGCTGA |
| 257 | 31C | 061827101-HERV0856uL_at3 | CCGAGTGGGCACATCAAGCACAGTG |
| 258 | 31A′ | 061827101-HERV0856uL_st1 | GTTGTCTGAAGGGCTGAAACTCTTC |
| 259 | 31B′ | 061827101-HERV0856uL_st2 | TCAGCTTGCGTGACAAACCTGGATC |
| 260 | 31C′ | 061827101-HERV0856uL_st3 | CACTGTGCTTGATGTGCCCACTCGG |
| 261 | 32A | 170828901-HERV0770cL_at1 | CACAGGTCTTGCCGAGACCCCCACG |
| 262 | 32B | 170828901-HERV0770cL_at2 | CCACGGGCTCACTGTTCAGCTCATC |
| 263 | 32C | 170828901-HERV0770cL_at3 | GCTCTGTCACAGTTTCCCACGACTT |
| 264 | 32A′ | 170828901-HERV0770cL_st1 | CGTGGGGGTCTCGGCAAGACCTGTG |
| 265 | 32B′ | 170828901-HERV0770cL st2 | GATGAGCTGAACAGTGAGCCCGTGG |
| 266 | 32C′ | 170828901-HERV0770cL st3 | AAGTCGTGGGAAACTGTGACAGAGC |
| 267 | 33A | 190148802-MALR1127uL_at1 | GGCCAAATTGTGCCACCCCTCCCAA |
| 268 | 33B | 190148802-MALR1127uL_at2 | AATTCCCAGGACCTCCTAATATGGC |
| 269 | 33C | 190148802-MALR1127uL_at3 | GGTCGTTGTAGGCCCAGCACAGTGG |
| 270 | 33A′ | 190148802-MALR1127uL_st1 | TTGGGAGGGGTGGCACAATTTGGCC |
| 271 | 33B′ | 190148802-MALR1127uL_st2 | GCCATATTAGGAGGTCCTGGGAATT |
| 272 | 33C′ | 190148802-MALR1127uL_st3 | CCACTGTGCTGGGCCTACAACGACC |
| 273 | 34A | 120093401-HERV1034uL_at1 | CCTAGCCATGAGCCAATTCCTTGCA |
| 274 | 34A′ | 120093401-HERV1034uL_st1 | TGCAAGGAATTGGCTCATGGCTAGG |

Another subject of the invention is the use of at least one, preferably at least 2, preferably at least 3, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, preferably at least 21, preferably at least 22, preferably at least 23, preferably at least 24, preferably at least 25, preferably at least 26, preferably at least 27, preferably at least 28, preferably at least 29, preferably at least 30, preferably at least 31, preferably at least 32, preferably at least 33, preferably at least 34 pairs of primers according to the invention, as described above,
and/or at least one, preferably at least 2, preferably at least 3, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, of preferably at least 17, preferably at least 18, preferably at least 19, preferably at least 20, preferably at least 21, preferably at least 22, preferably at least 23, preferably at least 24, preferably at least less 25, preferably at least 26, preferably at least 27, preferably at least 28, preferably at least 29, preferably at least 30, preferably at least 31, preferably at least 32, preferably at least 33, preferably at least 34 hybridization probes according to the invention, as described above,
to determine in vitro or ex vivo the immune status of an individual, preferably a patient.

Another subject of the invention is a method for determining in vitro or ex vivo the immune status of an individual, as described above, in which at least one, preferably at least 2, preferably at least 3, of preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, preferably at least 21, preferably at least 22, preferably at least 23, preferably at least 24, preferably at least 25, preferably at least 26, preferably at least 27, preferably at least 28, of preferably at least 29, preferably at least 30, preferably at least 31, preferably at least 32, preferably at least 33, preferably at least 34 pairs of primers as described above;
and/or at least one, preferably at least 2, preferably at least 3, preferably at least 4, preferably at least 5, preferably at least 6, preferably at least 7, preferably at least 8, of preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, preferably at least 21, preferably at least 22, preferably at least 23, preferably at least 24, preferably at least 25, preferably at least 26, preferably at least 27, preferably at least 28, preferably at least 29, preferably at least 30, preferably at least 31, preferably at least 32, preferably at least 33, of preferably at least 34 hybridization probes as described above, are used.

Another subject of the invention is a kit comprising means for amplifying and/or detecting at least one, preferably at least 2, preferably at least 3, preferably at least 4, preferably at least 5, preferably at least 6, preferably at least 7, preferably at least 8, preferably at least 9, preferably at least 10, preferably at least 11, preferably at least 12, preferably at least 13, of preferably at least 14, preferably at least 15, preferably at least 16, preferably at least 17, preferably at least 18, preferably at least 19, preferably at least 20, preferably at least 21, preferably at least 22, preferably at least 23, preferably at least 24, preferably at least 25, preferably at least 26, preferably at least 27, preferably at least 28, preferably at least 29, preferably at least 30, preferably at least 31, preferably at least 32, preferably at least 33, preferably at least 34 sequences selected from the sequences identified in SEQ ID NO: 1 to 34 or from the sequences which exhibit at least 99% identity with one of the sequences identified in SEQ ID NO: 1 to 34.

Preferably, in the kit according to the invention:
the amplification means comprise, preferably consist of at least one, preferably at least 2, preferably at least 3, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, preferably at least 21, preferably at least 22, preferably at least 23, preferably at least at least 24, preferably at least 25, preferably at least 26, preferably at least 27, preferably at least 28, preferably at least 29, preferably at least 30, preferably at least 31, preferably at least 32, preferably at least 33, preferably at least 34 pairs of primers as described above;
and/or the detection means comprise, preferably consist of, at least one, preferably at least 2, preferably at least 3, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, preferably at least 21, preferably at least 22, preferably at least 23, preferably at least 24, preferably at least 25, preferably at least 26, preferably at least 27, preferably at least 28, preferably at least 29, preferably at least 30, preferably at least 31, preferably at least 32, preferably at least 33, preferably at least 34 hybridization probes such as described above.

Also preferably, the kit according to the present invention further comprises means for amplifying and/or detecting other biomarkers, particularly endogenous biomarkers (or loci), such as other HERV/MaLR and/or genes, preferably genes involved in the inflammation and/or the immunity, and/or housekeeping genes, and/or exogenous biomarkers, such as viruses. Among the genes involved in the immunity, the following genes may be mentioned in particular, of particular interest:
CD74, CX3CR1, IL-10, S100A8, S100A9, MERTK, CLEC7A, CD36, TIMP2, CCL13, PTGS2, IL-12B, IL-6, IL-1A, CCL20, MX1, OAS-1, CCL15, OAS-3, EIF2AK2, IFNγ, NEFH, MMP10, SERPINB2, THBD, STAT1, CCR4, HLA-DRB1/B3, TCF7, EOMES, BCL11B, ITGA7, IL-18R1, NLRC4, CYP1B1, HGF, IL-5RA, CCLP4, CD3G, CD4OLG, CD3D, CD127, ICOS IL-1R2, IL-1RN, IL-18, IL-18RAP, OX40L, PD-1, PD-L1, Zonulin (HP), BTLA, C3AR1, CD154, GM-CSF, IFIH1, IL-15, MCP1, PCSK9, STAT4, LTR82B CIITA, LILRB2, CD177, ADGRE3, FLT-1, CD64, TREM-1, TNF-α, IL-1β, ALOX5, IL-17A, NFκB, TBX21, HIF1a, RORgT, OAS-2, GNLY, CTLA-4, TIM 3, CD274, IL-2, IL-7R, GATA3, CXCL10, FAS, GSN, MDC1, DYRK2, TDRD9, CNB1IP1, ZAP70 and ARL14EP.

Even more preferably, the kit according to the present invention comprises means for amplifying and/or detecting at most 100, preferably at most 90, preferably at most 80, preferably at most 70, preferably at most. plus 60, preferably at most 50, preferably at most 40, preferably at most 30, preferably at most 20, preferably at most 10 biomarkers, in total.

The present invention also concerns the use of a kit as described above, for determining in vitro or ex vivo the immune status of an individual, preferably a patient.

### FIGURES

FIG. 1: HERV-V3 chip analysis pipeline. This diagram represents the different steps required to analyze data from the HERV-V3 chip.

FIG. 2: FIGS. 2A and 2B respectively represent the level of expression at the protein and molecular (mRNA) levels of TNF-α and IL-10 produced by PBMCs, taken from 5 healthy volunteers, stimulated by LPS. The protein quantification was carried out by an ELISA test on culture supernatants obtained after completion of the endotoxin tolerance model. In FIG. 2A, the y-axis represents the protein concentrations (pg/mL) of TNF-α and IL-10. In FIG. 2B, the y-axis represents the expression levels of TNF-α and IL-10 (in expression ratio or Fold Change (FC)). The three conditions are represented, NS for the negative controls (without stimulation), LPS for the cells stimulated once with 100 ng/ml of LPS and ET for the cells subjected to two stimulations with LPS (2 ng/mL then 100 ng/ml). Paired Wilcoxon tests were performed for statistical analysis of the results: ***, means that the p-value>0.01 between 2 conditions (NS vs. LPS or NS vs. ET or LPS vs. ET). **, means that the p-value<0.05 between 2 conditions. *, means that the p-value>0.1 between 2 conditions.

FIG. 3: FIG. 3 shows the expression of the genes of TNF-α (A) and IL-10 (B), and of the sequences SEQ ID NO: 1 (C), SEQ ID NO: 4 (D), SEQ ID NO: 5 (E) and SEQ ID NO: 6 (F), by PBMCs, taken from 5 healthy volunteers, stimulated by LPS and quantified by biochips after the completion of the endotoxin tolerance model. The y-axis represents the fluorescence intensity of each of the hybridization probes: (A) 207113_s_at for the TNF alpha, (B) 207433_at for the IL-10, (C) 190665001-HERV0376 uL_at for SEQ ID NO: 1, (D) 121601801-HERV0492 uL_at for SEQ ID NO: 4, (E) 011052702-MALR1044 uL_at for SEQ ID NO: 5 and (F) 011052202-HERV1033 uL_at for SEQ ID NO: 6. The three conditions are represented, NS for the negative controls (without stimulation), LPS for cells stimulated once with 100 ng/ml of LPS and ET for cells subjected to two stimulations with LPS (2 ng/ml then 100 ng/ml).

FIG. 4: FIG. 4 shows the expression of the sequences SEQ ID NO: 1 (C), SEQ ID NO: 4 (D), SEQ ID NO: 5 (E), SEQ ID NO: 6 (F), by PBMCs, taken from 5 healthy volunteers, stimulated by LPS and quantified by RT-qPCR after completion of the endotoxin tolerance model. The y-axis represents the expression rates of the sequences mentioned above. The three conditions are represented, NS for negative controls (without stimulation), LPS for cells stimulated once with 100 ng/ml of LPS and ET for cells subjected to two stimulations with LPS (2 ng/ml then 100 ng/ml).

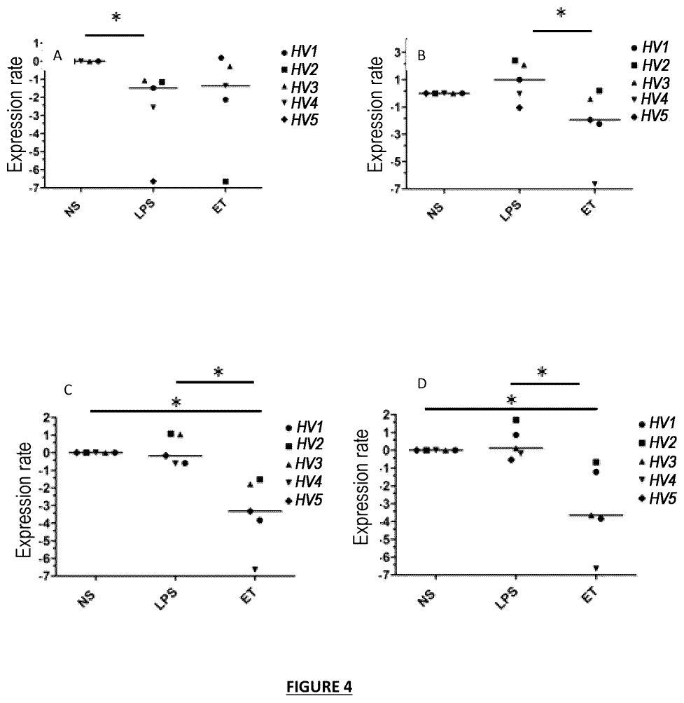

Paired Wilcoxon tests were performed for statistical analysis of the results: ***, means that the p-value<0.01 between 2 conditions (NS vs. LPS or NS vs. ET or LPS vs. ET). **, means that the p-value<0.05 between 2 conditions. *, means that the p-value<0.1 between 2 conditions.

FIG. 5: FIG. 5 shows the expression of the sequences SEQ ID NO: 2 (A), SEQ ID NO: 3 (B), SEQ ID NO: 7 (C), SEQ ID NO: 8 (D), SEQ ID NO: 11 (E) and SEQ ID NO: 12 (F), from whole blood of 20 patients in septic shock stratified according to the level of expression of mHLA-DR and quantified by biochips. The y-axis represents the fluorescence intensity of each of the hybridization probes: (A) 220247002-HERV0797 uL for SEQ ID NO: 2, (B) 170369402HE41env for SEQ ID

NO: 3, (C) 130360601-HERV0808cL for SEQ ID NO: 7, (D) 141107102-MALR1019 uL for SEQ ID NO: 8, (E) 050286701-HERV0513 uL for SEQ ID NO: 11 and (F) 050287402-MALR1022 uL for SEQ ID NO: 12. The three conditions are represented, HV for healthy volunteers, and on D1 and D3, DR+for patients, having a high expression of HLA-DR, considered as immunocompetent and DR-patients, having a low expression of HLA-DR, considered as immunodepressed.

FIG. 6: FIG. 6 shows the expression of the sequences SEQ ID NO: 2 (A), SEQ ID NO: 3 (B), SEQ ID NO: 7 (C), SEQ ID NO: 8 (D), SEQ ID NO: 11 (E) and SEQ ID NO: 12 (F), from whole blood of 20 patients in septic shock stratified according to the level of expression of mHLA-DR and quantified by RT-qPCR. The y-axis represents the expression rates of the sequences mentioned above. The three conditions are represented, HV for healthy volunteers, and on D1 and D3, DR+ for patients, having a high expression of HLA-DR, considered as immunocompetent and DR-patients, having a low expression of HLA-DR, considered as immunodepressed.

FIG. 7: FIG. 7 shows the expression of the sequences SEQ ID NO: 9 (A) and SEQ ID NO: 10 (B), from whole blood of 102 patients in septic shock stratified according to the ratio between the level of expression of CD74 on D3 and the expression level of CD74 on D1 and quantified by biochips. The y-axis represents the fluorescence intensity of each of the hybridization probes: (A) 021460102-HERV0599 uL_st for SEQ ID NO: 9 and (B) 021456001-MALR1017 uL_at for SEQ ID NO: 10. The following conditions are represented on D1, D3 and D6, immunocompetent patients («high» CD74 D3/D1 ratio) and immunodepressed patients («low» CD74 D3/D1 ratio).

FIG. 8: FIG. 8 shows the expression of the sequences SEQ ID NO: 9 (A) and SEQ ID NO: 10 (B), from the whole blood of 102 patients in septic shock stratified as a function of the ratio between the level of expression of CD74 on D3 and the expression level of CD74 on D1 and quantified by RT-qPCR. The y-axis represents the expression rates of the sequences mentioned above. The following conditions are represented on D1, D3 and D6, immunocompetent patients («high» CD74 D3/D1 ratio) and immunodepressed patients («low» CD74 D3/D1 ratio).

FIG. 9: FIG. 9 shows a graph representing the association between the size of the signature of markers and the discriminating power between patients considered to be immunocompetent and those considered to be immunodepressed.

The present invention is illustrated in a non-limiting manner by the following examples.

### EXAMPLE 1: ENDOTOXIN TOLERANCE (ET) MODEL

The endotoxin tolerance corresponds to a temporary state of the inability of a cell or organism to respond to endotoxin stimulation, resulting from a first endotoxin stimulation.

The endotoxin tolerance model was set up to mimic on the one hand an inflammatory context induced by a stimulation by lipopolysaccharide (LPS) and on the other hand the monocytic anergy which represents a state of «no response» of the cells. These components of immunity are found in different types of patients, such as patients suffering from sepsis, trauma, burns or who have undergone major surgery.

### Materials and Methods

### PBMC Stimulation

The endotoxin tolerance model was established from 5 bags of citrated blood from healthy volunteers collected at the EFS in accordance with standard procedures for blood donation, and used immediately after receipt. PBMCs, peripheral blood mononuclear cells, are isolated using a density gradient adjusted to 2 million cells per mL and cultured in X-Vivo medium (Lonza) at 37° C. and 5% CO2. The endotoxin used in this model is the lipopolysaccharide (LPS), which is a major component of the outer membrane of Gram negative bacteria. The LPS is obtained from a mixture of three strains of Escherichia coli: O111: B4, O55: B5 and O127: B8 (Sigma). All of the conditions are carried out in biological triplicates. In this ex-vivo model of endotoxin tolerance, the PBMCs are first cultured for 15 hours without (NS control cells and LPS cells mimicking the inflammatory condition) or with a dose of 2 ng/ml of LPS (ET cells mimicking monocytic anergy, immunosuppression condition). After a washing step, the PBMCs are incubated a second time for 6 hours without (NS control cells) or with a dose of 100 ng/ml of LPS (LPS and ET cells).

At the end of the experiments, the culture supernatants are harvested and stored at −80° C. The cells are also harvested, lysed and stored at −80° C. before performing RNA extraction (Qiagen commercial kit) for the transcriptomic study. To validate the efficiency of the model, the concentrations of the pro-inflammatory cytokine TNF-α («tolerizable gene») and of the anti-inflammatory cytokine IL10 («non-tolerizable gene») are determined from the culture supernatants of PBMC by ELISA (commercial R&D System kits).

### RNA Extraction and Amplification

The mRNAs are extracted using commercial kits (RNeasy Mini Plus kit, QIAGEN) from the ex vivo cultures of PBMCs described above.

The total mRNAs are then assayed and characterized. The characterization of the mRNA is carried out by capillary electrophoresis using the Bioanalyser 2100. The quality of the mRNA samples is evaluated by the calculation of the RIN (RNA Integrity Number). This value is based on the detection of 18S and 28S ribosomal RNAs, if the RIN tends towards 10 it means that the mRNA is intact (a RIN≥7 is accepted).

The synthesis of the complementary DNA (cDNA) and the amplification steps are carried out by a linear and isothermal method described in 2005 by the company NuGEN Technologies (Kurn N et al. Novel isothermal, linear nucleic acid amplification systems for highly multiplexed applications. Clinical chemistry. 2005; 51 (10): 1973-81). The Ribo-SPIA amplification process is used (Watson J D et al. Complementary RNA amplification methods enhance microarray identification of transcripts expressed in the C. elegans nervous system. BMC genomics. 2008; 9:84) from 16 ng of Total RNA (WTO pico commercial kit, Nugen) and consists of three steps. The first step is the production of the first strand of cDNA by reverse transcription from an mRNA template, this by using a mixture of random primers and oligo-dT. The second step, consists in adding DNA polymerase to the reaction, which induces the production of the second strand of cDNA. The third step, involves the SPIA amplification by strand displacement. Hybrid DNA/RNA primers are degraded by the RNAse H activity of DNA polymerase when complexed with the cDNA template. Single-stranded DNA synthesis (complementary to the mRNA template) is initiated and continues, authorizing new SPIA primers to be fixed to the cDNA template, thus sustaining the repetitive process of strand synthesis. The cDNAs are then fragmented into fragments of 50-200 bp using a DNase from 5 μg of purified and amplified DNA (commercial kit, Nugen) and are also labeled in 3′ (Nugen commercial kit). The amplification and fragmentation of the cDNA are verified on the Bioanalyser. The amplification profile spans a cDNA size range from 25 to 4000 nucleotides, with a peak around 1500 pb. The fragmentation profile should be centered on a nucleic acid population around 100 nucleotides in size, which is recommended for hybridization on an Affymetrix microarray.

### Analysis by Biochip

The identification of sequences exhibiting an expression differential is based on the design and the use of a high density DNA chip in GeneChip format, called HERV-V3, designed by the inventors and whose manufacture has been subcontracted to the Affymetrix company. This chip contains probes that are hybridized to distinct HERV sequences within the human genome. These sequences are extracted from a database specific to the inventors, already published (Becker et al. A comprehensive hybridization model allows whole HERV transcriptome profiling using high density microarray, BMC Genomics 2017 18:286).

The HERV-V3 chip targets 353,994 HERV/MaLR elements, and over 1,500 immunity genes.

Once the cDNAs have been amplified and fragmented, they can be hybridized on the HERV-V3 chip, in an oven at 50° C. for 18 hours with constant stirring at 60 rpm for hybridization. A fluidics system allows automating the washing and coloring steps, and finally after all of these steps, the chip is read using a fluorometric scanner.

The raw dataset is created from the aggregation of the CEL files of each chip by traditional Affymetrix methods.

After a first quality control of the raw data, several steps are carried out: correcting the background noise by the Robust Multi-array Average (RMA) method, standardizing the data of each chip by quantiles, grouping the probe data into sets of probes (probes in probesets) and smoothing of the median. A second step of quality control is carried out. All these steps thus make it possible to obtain a matrix containing the normalized data.

The pre-treatment of the chips as well as the statistical analysis are carried out by using R/Bioconductor.

A pre-analysis step consists on evaluating the quality of the chips, before and after normalization. For this, several criteria must be taken into account: the quality of the RNA, the amplification and fragmentation controls of the cDNA, the image of the chips produced after the scan, the hybridization of the Affymetrix controls, the signal intensity (before and after normalization), probeset homogeneity (RLE and NUSE plots), chips correlation (before and after normalization) and principal component analysis. For all of these criteria, a statistical analysis allows identifying the extreme values for each chip and the data are then grouped together. Chips that pass less than 5 quality controls are removed from the analysis. For each data set, a decision table is then produced to summarize all the quality criteria, and quickly identify the chips to be removed from the analysis.

Sometimes it is necessary to have a corrective method for the analysis of data. This so-called COMBAT correction (for Combining BATches) makes it possible to correct the technical variability of the dataset and thus bring out its biological variability.

Finally, a data filter step was performed to reduce the data set and gain statistical power for the analyses. The intensity threshold was defined as the minimum intensity value for which the 75^{th} percentile of the distribution of the coefficients of variation is below 10%. In this way, the intensity threshold is 2^{5.5}. The probesets below the intensity threshold in more than 68% of all samples (31 of 45 samples) are eliminated.

Among the 71,063 probesets targeting HERVs/MaLRs and the 42,560 probesets targeting genes selected in the previous step, a differential expression analysis was performed.

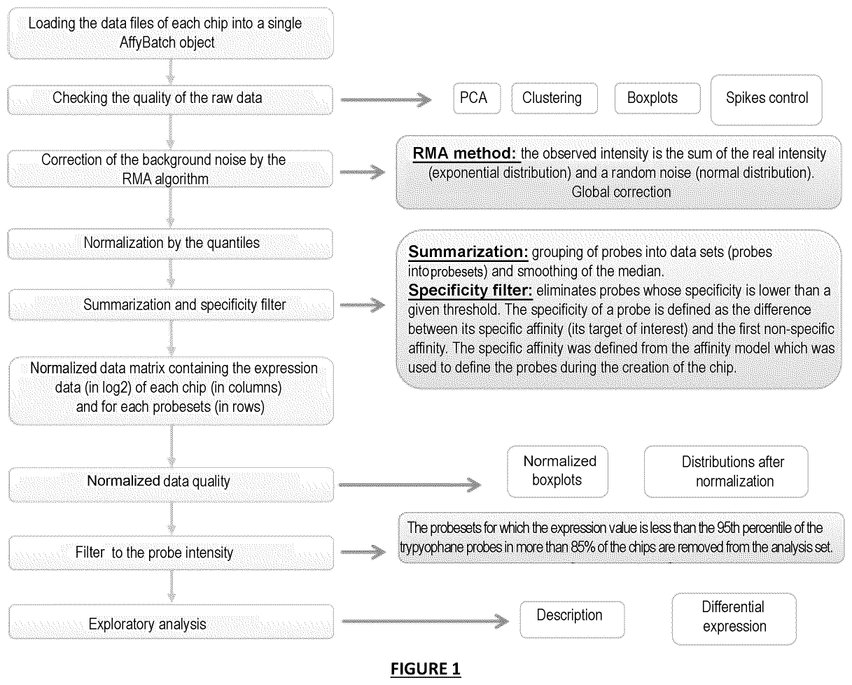

Studying the differential expression between two conditions is like calculating the expression ratio or Fold Change (FC). For example an expression value equal to 10 under condition A, and a value equal to 5 under condition B, the FC of A/B is equal to 2. The FC data will be represented in log 2 FC. To determine that a gene or a HERV/MaLR sequence is differentially expressed between two conditions, the Limma method was used (Smyth G K. Linear models and empirical bayes methods for assessing differential expression in microarray experiments. Statistical applications in genetics and molecular biology 2004, 3:Article3). Statistical tests and their associated p-value are calculated to evaluate the significance of the observed expression changes. The p-values were adjusted by controlling the false discovery rate (FDR, due to multiple tests) according to the method of Benjamini and Hochberg (Hochberg et al. Controlling the False Discovery Rate: A Practical and Powerful Approach to Multiple Testing. Journal of the Royal Statistical Society Series B (Methodological) 1995, Vol. 57, No. 1 (1995), pp. 289-300). A probeset is considered to be significantly differentially expressed when the absolute value of FC in log 2 is greater than 1 and the fitted p-value is below 0.05 (see FIG. 1).

The sequences which were identified as being the most differentially expressed by the biochip were validated by RT-qPCR from the same samples as those which made it possible to produce the HERV-V3 chips.

### Results

As indicated above, to validate the efficiency of the model, the concentrations of the pro-inflammatory cytokine TNF-α («tolerizable gene») and of the anti-inflammatory cytokine IL10 («non-tolerizable gene») are determined from the supernatants of PBMC culture by ELISA (commercial R&D System kits).

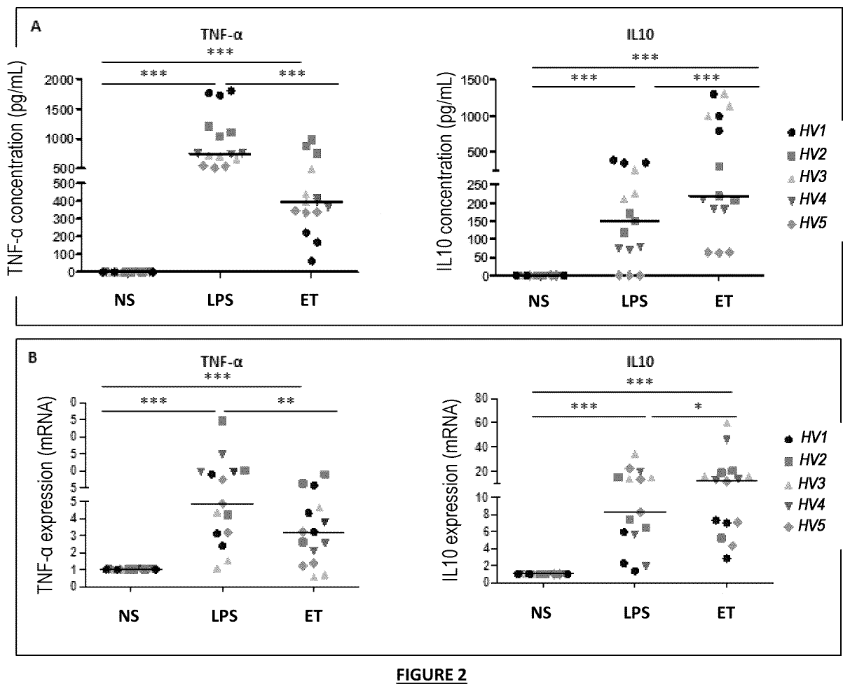

As shown in FIG. 2A, cells stimulated twice with LPS (ET model, immunosuppression condition) produce low amounts of TNF-α (100-500 pg/mL) compared to cells stimulated only once (LPS model, inflammatory condition) (500-2000 pg/mL). In contrast, these same cells secrete higher concentrations of IL-10 (100-1000 pg/mL) compared to cells stimulated once (50-400 pg/mL). These results at the protein level are confirmed at the mRNA level since a significant decrease in the expression of TNF-α coupled with an increase in the gene expression of IL-10 (FIG. 2B) in the cells stimulated twice by LPS were observed in comparison to once-stimulated PBMCs.

These results allow validating the effectiveness of this model.

An analysis of the expression of HERV/MalR and genes in unstimulated PBMC (NS, control), stimulated once with LPS (LPS, inflammatory condition) or twice stimulated with LPS (ET, immunosuppression condition) was made on the HERV-V3 biochip, designed by the inventors.

The processing of the data generated by the analysis of the HERV-V3 chips using this method made it possible to identify a set of 4 sets of probes (or «probesets»). These «probesets» are the most statistically differentially expressed among the sequences showing a statistically significant difference in expression between the different conditions (healthy patients, inflammatory condition and immunosuppression condition). As indicated previously, for the expression differential to be statistically significant, the absolute value of log 2 Fold Change must be greater than or equal to 1 and the adjusted p-value must be less than or equal to 0.05. These criteria will apply for all examples. These 4 «probesets» are associated with HERV sequences identified by SEQ ID NO: 1 and 4 to 6. The chromosomal location of each sequence is given in the GRCh38 reference. In Table 9, below, the list of identified sequences.

TABLE 9

| | NAME OF THE | | GRCH38 LOCATION |
|---|---|---|---|
| SEQ | PROBESETS OF THE | FAMILY | OF THE ENTIRE |
| ID NO: | HERV-V3 CHIP | NAME | ELEMENT |
| SEQ ID | 190665001-HERV0376 | LTR101 | chr19:54891074- |
| NO: 1 | | | 54891496 |
| SEQ ID | 121601801-HERV0492 | LTR33 | chr12:112971073- |
| NO: 4 | | | 112971451 |
| SEQ ID | 011052702-MALR1044 | MSTC | chr12:112971073- |
| NO: 5 | | | 112971451 |
| SEQ ID | 011052202-HERV1033 | MLT2B5 | chr1:78623489- |
| NO: 6 | | | 78623954 |

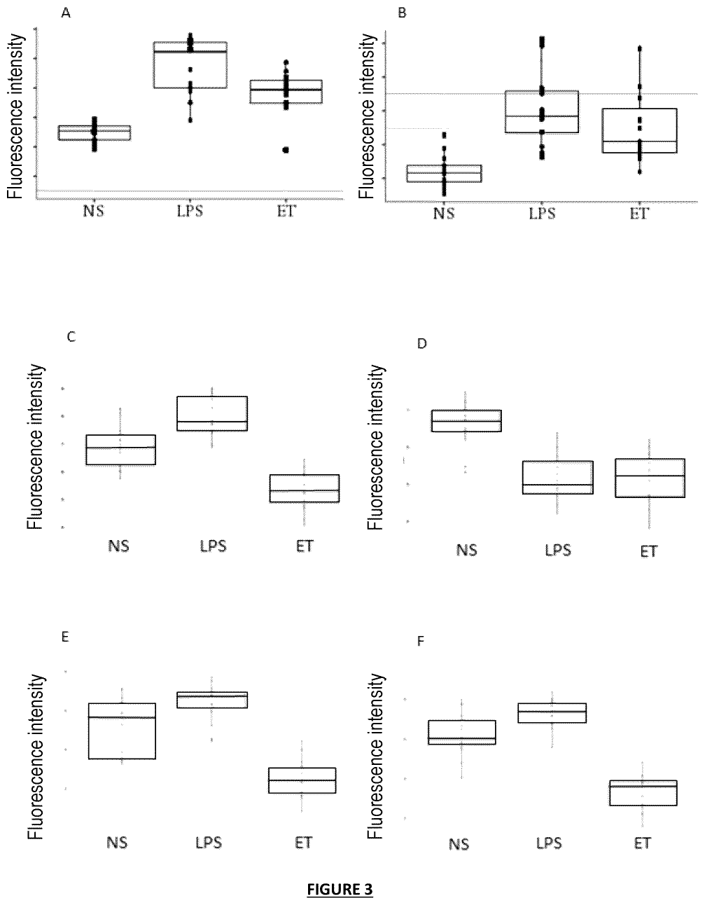

As observed in FIG. 3C, the expression of the sequence SEQ ID NO: 1 is statistically significantly greater under the NS condition compared to the LPS and ET conditions.

FIGS. 3D and 3F illustrate the expression profile of the sequences SEQ ID NO: 4 and SEQ ID NO: 5 observed in the PBMCs with the HERV-V3 chip. These figures show a similar expression profile, namely, for these two sequences, an increase in expression under the LPS condition compared to the NS and ET conditions, with a statistically significant difference between LPS and ET.

The sequence SEQ ID NO: 6, for its part, has greater expression under the NS and LPS conditions compared to the ET condition, with statistically significant differences between NS and ET, and between LPS and ET (FIG. 3F).

These results show a modulation of the expression of these sequences in the endotoxin tolerance model and thus their ability to be used as a marker of immune status.

The results also show that the sequences SEQ ID NO: 4 to 6 have a «tolerizable» profile (inflammatory condition) in an endotoxin tolerance model while the sequence SEQ ID NO: 1 has a «non-tolerizable» profile (immunosuppression condition) in an endotoxin tolerance model.

FIG. 3 illustrates the expression of the sequences SEQ ID NO: 1 (C), SEQ ID NO: 4 (D), SEQ ID NO: 5 (E), SEQ ID NO: 6 (F), on PBMCs, obtained from the same 5 healthy volunteers than for the HERV-V3 biochip, stimulated by LPS and quantified by RT-qPCR after completion of the endotoxin tolerance model.

The results show that the same profile as that obtained on the biochips is observed for each identified sequence. Thus, the data obtained by the HERV-V3 biochip are confirmed by RT-qPCR (FIG. 3).

### EXAMPLE 2: PATIENTS IN INTENSIVE CARE

### Materials and Methods

### Patients and Biological Samples

This retrospective observational study was conducted in patients aged 37 to 77 years (13 men, 7 women, median age: 59 years) admitted to intensive care following septic shock.

Whole blood samples were collected in PAXgene tubes (PreAnalytix) from these 20 patients in septic shock on days 1 (D1) and 3 or 4 (D3) after admission to intensive care, and were then stored (retrospective cohort).

Patients in this cohort were stratified according to the expression level of HLA-DR at the surface of monocytes (mHLA-DR). The expression of mHLA-DR was measured on D3 (days 3 or 4) by flow cytometry.

20 patients from this cohort were selected, 10 patients (50%) had high expression of HLA-DR on day 3 or 4 (more than 30% of expression), and 10 patients (50%) had a low expression of HLA-DR on day 3 or 4 (less than 30% of expression).

Patients with high expression of HLA-DR are considered to have immunocompetent status (DR+) and patients with low expression of HLA-DR are considered to have immunodepressed status (DR−).

5 healthy volunteers are also included in this study.

### RNA Extraction

RNA extraction was performed using the PAXgene Blood RNA Kit (PreAnalytix) following the manufacturer recommendations. Before the RNA elution step, residual genomic DNA was removed by the action of DNAse. The RNA concentration was determined by fluorimetry (RNA assay kit on Qubit, Life Technologies). The quality of the RNA was then controlled using the RNA 6000 Nano kit on a Bioanalyzer (Agilent Technologies), the collections with a RIN (RNA Integrity Number) greater than 6 being considered to be of good quality.

The steps of RNA amplification, biochip analysis and validations of sequences by RT-qPCR which were identified as being differentially expressed by the biochip are carried out as described in Example 1.

### Results

The processing of the data generated by the analysis of the HERV-V3 chips using this method made it possible to identify a set of 6 sets of probes (or «probesets»). These «probesets» are the most statistically differentially expressed among the sequences showing a statistically significant difference in expression between the two conditions (immunocompetent condition and immunosuppression condition). These 6 «probesets» are associated with HERV sequences identified by SEQ ID NO: 2, 3, 7, 8, 11 and 12. The chromosomal location of each sequence is given in the GRCh38 reference. In Table 10, below, the list of the 6 identified sequences.

TABLE 10

| | NAME OF THE | | |
|---|---|---|---|
| | PROBESETS | | |
| SEQ | OF THE | FAMILY | GRCH38 LOCATION OF |
| ID NO: | HERV-V3 CHIP | NAME | THE ENTIRE ELEMENT |
| SEQ ID | 220247002- | MER4B | chr22:36153696-36154283 |
| NO: 2 | HERV0797 | | |
| SEQ ID | 170369402HE41env | HERV-E4.1 | chr17:35505737-35508365 |
| NO: 3 | | | |
| SEQ ID | 130360601- | MER50C | chr13:42884951-42886257 |
| NO: 7 | HERV0808 | | |
| SEQ ID | 141107102- | MLT1J1 | chr14:91230494-91230820 |
| NO: 8 | MALR1019 | | |
| SEQ ID | 050286701- | LTR40A | chr5:14551189-14551685 |
| NO: 11 | HERV0513 | | |
| SEQ ID | 050287402- | MLT1K | chr5:14562791-14563322 |
| NO: 12 | MALR1022 | | |

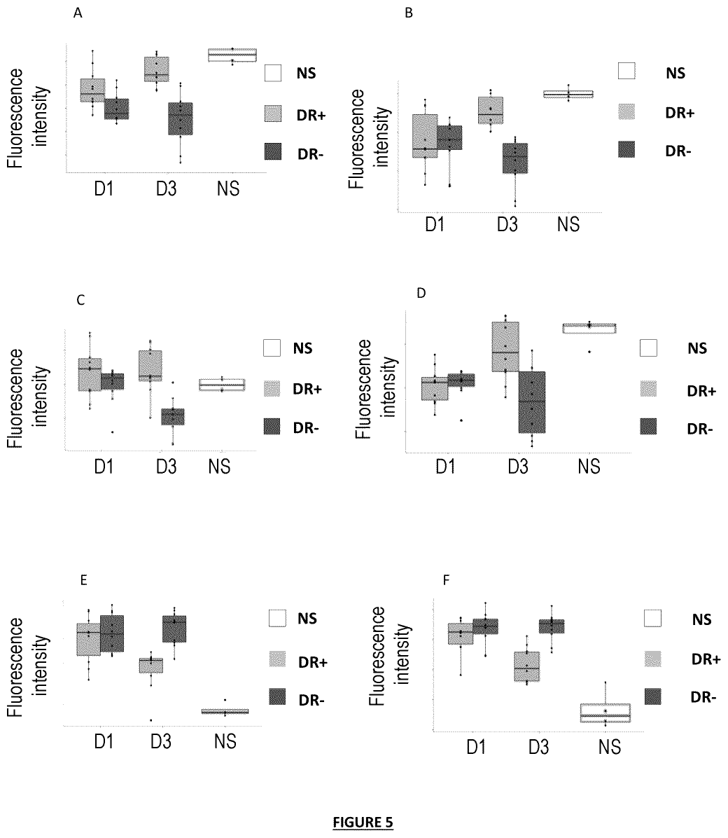

As observed in FIGS. 5A and 5C, the expression of the sequences SEQ ID NO: 2 and 7 is decreased in immunodepressed patients (DR−) from D1 after admission to intensive care compared to immunocompetent patients. While the expression of SEQ ID NOs: 3, 8, 11 and 12 is decreased in immunodepressed patients (DR−) on D3 after admission to intensive care compared to immunocompetent patients (see FIGS. 5B, 5D, 5E and 5F).

Thus, these results show the usefulness of the sequences SEQ ID NO: 2 and 7 as markers, from D1, of immunosuppression. The sequences SEQ ID NO: 3, 8, 11 and 12, for their part, are markers of immunosuppression at D3.

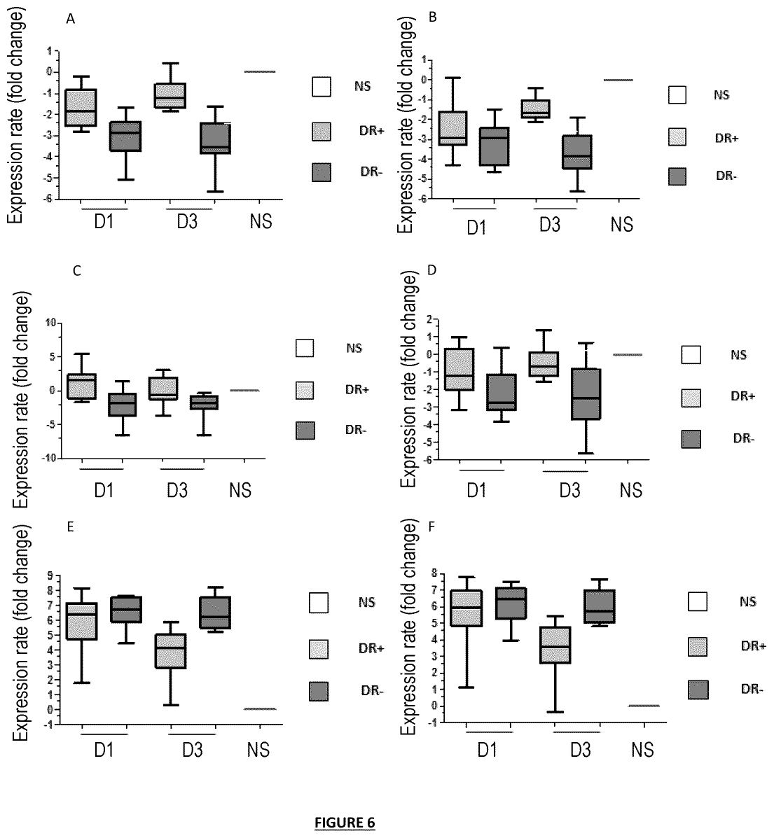

These sequences, identified as being differentially expressed by the biochip, were validated by RT-qPCR. The results are illustrated in FIG. 6.

Profiles similar to those obtained on the biochips are observed for each identified sequence. Thus, the data obtained by the HERV-V3 biochip are confirmed by RT-qPCR.

### EXAMPLE 3: PATIENTS IN RESUSCITATION

### Materials and Methods

### Patients and Biological Samples

This retrospective observational study was conducted in patients admitted to resuscitation in 6 French hospitals from 2009 to 2011. The inclusion criteria were as follows:
patients aged 18 or over;
prediction by the clinician of a length of stay in intensive care of at least 2 days;
patients having at least one site of acute infection suspected or confirmed by the clinician on clinical or paraclinical manifestations;
patients having at least two of the following criteria:
temperature above 38° C. or below 36° C.;
heart rate greater than 90 beats per minute;
respiratory rate greater than 20 breaths per minute or PaCO2<32 mmHg;
number of leukocytes greater than 12000/mm3 or less than 4000/mm3.

The exclusion criteria were as follows:
pre-existing immunodepression, including recent chemotherapy or immunosuppressive treatment,
a high dose (>5 mg/kg of prednisolone equivalent for a duration>5 days) or a prolonged treatment (0.5 mg/kg of prednisolone>30 days) of corticosteroids;
aplasia (circulating neutrophils<500 cells/mm3),
primary immunodeficiency and
extracorporeal circulation the month preceding admission to the intensive care unit.

Among all patients, 102 patients met the following criteria:
patients with septic shock;
a first blood collection was taken within the first 24 hours of the patient arrival in intensive care (D1);
a second blood collection was taken between 3 and 4 days following the patient arrival in intensive care (D3);
a third blood collection was taken 6 days after the patient arrival in intensive care (D6).

Then, the patients of this cohort were stratified according to the ratio of the level of expression of CD74 on D3 to the level of expression of CD74 on D1, carried out by RT-qPCR.

The cohort was divided into 2 categories. People with a CD74 D3/D1 ratio greater than 1.23 are called «high» and are considered to have an immunocompetent status. Those with a CD74 D3/D1 ratio of less than 1.23 are called «low» and are considered to have an immunodepressed status.

Among the 102 patients in this cohort, 52 patients (51%) are considered to have an immunodepressed status («low» CD74 D3/D1 ratio) and 50 patients (49%) are considered to have an immunocompetent status («high» CD74 D3/D1 ratio).

The RNA extraction step is implemented as described in Example 2.

The steps of amplifying the RNA, analyzing by biochip and validating by RT-qPCR (on D1 and D3) the sequences which have been identified as being differentially expressed by the biochip are implemented as described in Example 1.

### Results

The processing of the data generated by the analysis of the HERV-V3 chips using this method made it possible to identify a set of 2 sets of probes (or «probesets»). These «probesets» are the most statistically differentially expressed among the sequences showing a statistically significant difference in expression between the two conditions (immunocompetent condition and immunosuppression condition). These 2 «probesets» are associated with HERV sequences identified by SEQ ID NO: 9 and 10. The chromosomal location of each sequence is given in the GRCh38 reference. In Table 11, below, the 2 identified sequences.

TABLE 11

| | NAME OF THE | | |
|---|---|---|---|
| | PROBESETS | | |
| SEQ ID | OF THE | FAMILY | GRCH38 LOCATION OF |
| NO: | HERV-V3 CHIP | NAME | THE ENTIRE ELEMENT |
| SEQ ID | 021460102- | LTR82B | chr2:102363654-102366601 |
| NO: 9 | HERV0599uL | | |
| SEQ ID | 021456001- | MLT1I | chr2:102013616-102013971 |
| NO: 10 | MALR1017uL | | |

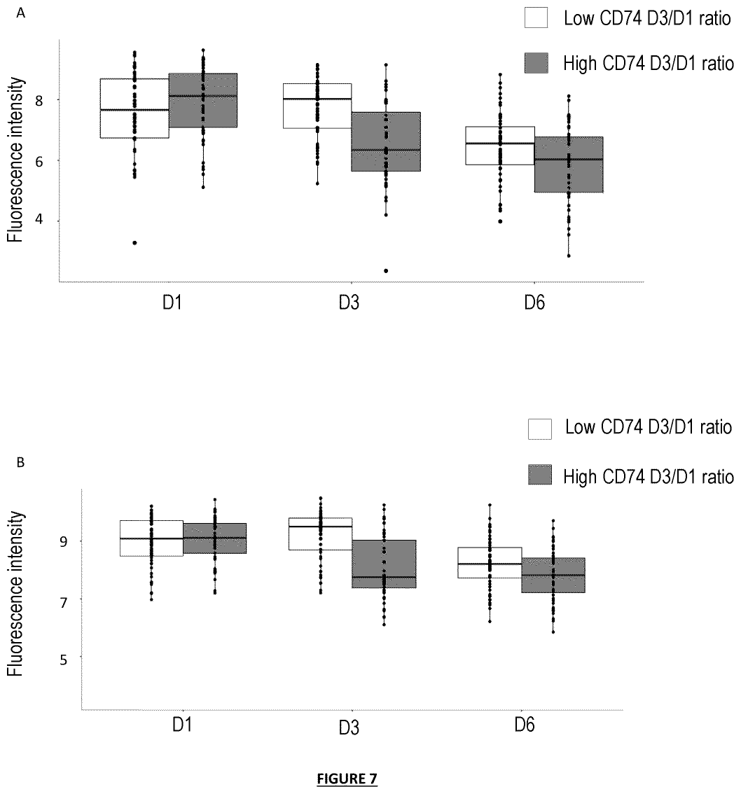

As observed in FIG. 7A, the expression of the sequence SEQ ID NO: 9 is higher on D3 in patients with a «low» CD74 D3/D1 ratio compared to patients with a «high» CD74 D3/D1 ratio.

Identically for the expression of the sequence SEQ ID NO: 10 (FIG. 7B), it is higher on D3 in patients with a «low» CD74 D3/D1 ratio compared to patients with a «high» CD74 D3/D1 ratio.

Thus, these results show the usefulness of these SEQ ID NOs: 9 and 10 sequences as a marker of the immunosuppression state on D3.

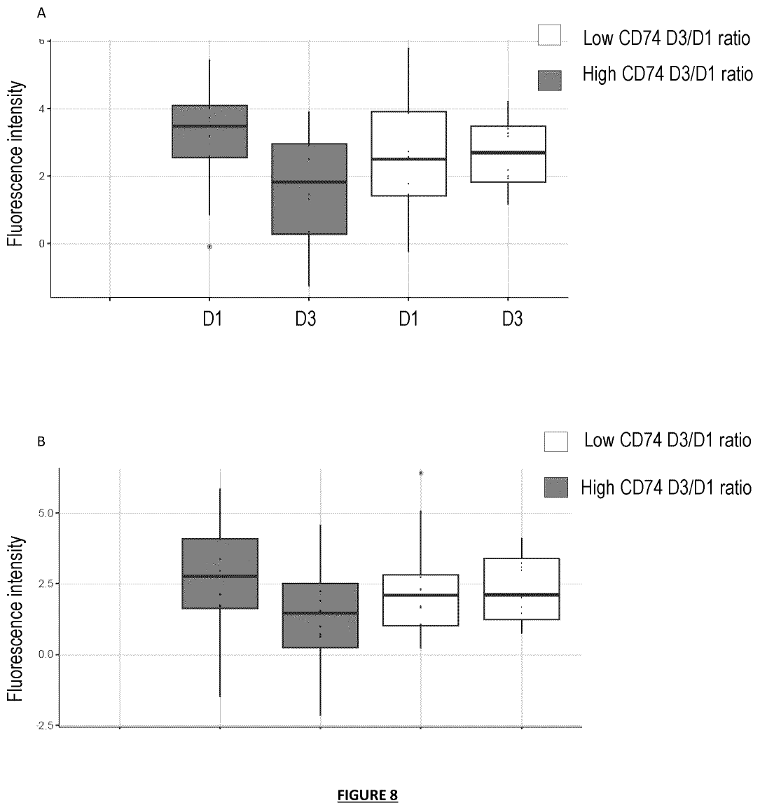

These sequences, identified as being differentially expressed by the biochip, were validated by RT-qPCR on D1 and D3 from 30 patients out of the 102 of the cohort. The results are illustrated in FIG. 8.

Profiles similar to those obtained on the biochips are observed for each identified sequence. Thus, the data obtained by the HERV-V3 biochip are confirmed by RT-qPCR from 30 patients.

### EXAMPLE 4: NOTATION OF IDENTIFIED SEQUENCES

For each of the sequences identified in Examples 1 to 3 (SEQ ID NO: 1-12), the inventors have assigned a rating. This rating is based on the expression profiles, in chips, of the sequences SEQ ID NO: 1 to 12 observed on the cohort of Example 2.

The inventors have assigned ratings ranging from 1 to 4 stars. All the criteria are visual, from graphs showing the expression of the different identified sequences.

As seen previously, all these sequences have already been selected on the basis of the level of expression and the differential of expression. The method of assigning ratings is described in Table 12, below.

TABLE 12

| CRITERIA | RATING |
|---|---|
| Increase of the expression in patients | **** |
| with low DR on D3 and visible | |
| difference from D1 | |
| Increase of the expression in patients | *** |
| with low DR on D3, difference not | |
| visible on D1 | |
| Decrease of the expression in patients | ** |
| with low DR from D1, difference | |
| visible or not at D3 | |
| Decrease of the expression in patients | * |
| with low DR on D3, difference | |
| not visible on D1 | |

Here in Table 13, below, the rating obtained for each of the sequences identified in Examples 1 to 3.

TABLE 13

| | NAME OF THE | | |
|---|---|---|---|
| SEQ ID | PROBESETS OF | CHROMOSOMAL | |
| NO: | THE HERV-V3 CHIP | LOCATION | SCORE |
| SEQ ID | 190665001-HERV0376 | chr19:54891074-54891496 | *** |
| NO: 1 | | | |
| SEQ ID | 220247002-HERV0797 | chr22:36153696-36154283 | ** |
| NO: 2 | | | |
| SEQ ID | 170369402HE41env | chr17:35505737-35508365 | * |
| NO: 3 | | | |
| SEQ ID | 121601801-HERV0492 | chr12:112971073-112971451 | ** |
| NO: 4 | | | |
| SEQ ID | 011052702-MALR1044 | chr1:78648318-78648697 | ** |
| NO: 5 | | | |
| SEQ ID | 011052202-HERV1033 | chr1:78623489-78623954 | ** |
| NO: 6 | | | |
| SEQ ID | 130360601-HERV0808 | chr13:42884951-42886257 | *** |
| NO: 7 | | | |
| SEQ ID | 141107102-MALR1019 | chr14:91230494-91230820 | * |
| NO: 8 | | | |
| SEQ ID | 021460102-HERV0599 | chr2:102363654-102366601 | *** |
| NO: 9 | | | |
| SEQ ID | 021456001-MALR1017 | chr2:102013616-102013971 | *** |
| NO: 10 | | | |
| SEQ ID | 050286701-HERV0513 | chr5:14551189-14551685 | **** |
| NO: 11 | | | |
| SEQ ID | 050287402-MALR1022 | chr5:14562791-14563322 | **** |
| NO: 12 | | | |

### EXAMPLE 5: HERV MARKERS OF THE IMMUNOSUPPRESSION

For the identification of markers specific to immunosuppression, the inventors used the data generated by the analysis of the HERV-V3 chips from the samples of Example 2.

Thus, the inventors selected the HERV sequences differentially expressed on D3 between the patients considered to be immunodepressed (DR−) and those considered to be immunocompetent (DR+), but which were not differentially expressed between the healthy volunteers of the study and all the patients (whether DR+ or DR−, whether on D1 or D3).

This selection allowed the identification of 17 HERV sequences. For each of the identified sequences, the inventors assigned a score according to several criteria (expression profile, fold change, level of expression, consistency of the expression profiles between the sense and antisense probes, difference in expression visible from D1 and absence of too much variability between patients). Below Table 14 describes the mode of attributing points for each criterion.

TABLE 14

| CRITERIA | DESCRIPTION | POINTS |
|---|---|---|
| Expression | Increase in patients considered to be | 2 |
| profile | immunocompetent (DR−) | or |
| | compared to those considered to | 1 |
| | be immunodepressed (DR+) | |
| | or | |
| | Decrease in patients considered to be | |
| | immunocompetent (DR−) | |
| | compared to those considered to | |
| | be immunodepressed (DR+) | |
| Level of | Level of expression generally above 5 | 1 |
| expression | | |
| Fold Change | High fold change (log2 FC | 1 |
| | visually greater than 1.5) | |
| Sense/antisense | Same expression profile between | 1 |
| consistency | sense and antisense probes | |
| Difference | Difference between high DR and | 1 |
| visible from D1 | low DR patients visible from D1 | |
| | (even if the difference is small). | |
| Variability | Penalty if the variability is too high. | −1 |

| | |
|---|---|
| SCORE | /6 |

Thus, thanks to this mode of attributing a score, the inventors selected 10 candidates and excluded 7 candidates whose performances seemed insufficient. Here in Table 15, below, the 10 markers identified with their respective score.

TABLE 15

| SEQ | NAME OF THE | | |
|---|---|---|---|
| ID | PROBESETS OF | CHROMOSOMAL | |
| NO: | THE HERV-V3 CHIP | LOCATION | SCORE |
| SEQ ID | 052182701-MALR1129 | chr5:132453630-132454148 | 5 |
| NO: 13 | | | |
| SEQ ID | 190478501-MALR1003 | chr19:41812466-41813010 | 4 |
| NO: 14 | | | |
| SEQ ID | 011790601ERV9sLU5 | chr1:155637287-155637547 | 4 |
| NO: 15 | | | |
| SEQ ID | 052681601-MALR1018 | chr5:170290289-170290812 | 4 |
| NO: 16 | | | |
| SEQ ID | 160627301-MALR1014 | chr16:50662453-50662912 | 4 |
| NO: 17 | | | |
| SEQ ID | 111686702-HERV0861 | chr11:122671887-122672147 | 3 |
| NO: 18 | | | |
| SEQ ID | 141107102-MALR1019 | chr14:91230494-91230820 | 3 |
| NO: 8 | | | |
| SEQ ID | 040318302-MALR1134 | chr4:15825146-15825565 | 2 |
| NO: 19 | | | |
| SEQ ID | 041529101-MALR1026 | chr4:83464568-83464963 | 2 |
| NO: 20 | | | |
| SEQ ID | 141106902-MALR1133 | chr14:91222760-91223118 | 2 |
| NO: 21 | | | |

The sequences SEQ ID NO: 13 to 18 and 19 to 21 are newly identified sequences. As for the sequence SEQ ID NO: 8, this is a sequence which has already been identified in Example 2.

### EXAMPLE 6: HERV MARKERS OF INFLAMMATION

For the identification of specific markers of inflammation, the inventors used the data generated by the analyses of the HERV-V3 chips from the samples of Examples 1 and 2.

Thus, the inventors selected the HERV sequences on the one hand differentially expressed between the LPS condition (immunosuppression condition) and the NS condition (negative controls), in Example 1, and on the other hand differentially expressed between the patients on D1 or D3 compared to healthy volunteers, of Example 2.

This selection made it possible to identify 13 HERV sequences. As in Example 5, for each of the identified sequences, the inventors assigned a score based on the same criteria. The mode of attributing points for each criterion is described in Table 16, below.

TABLE 16

| CRITERIA | DESCRIPTION | POINTS |
|---|---|---|
| Expression | Increase in patients compared to | 2 |
| profile | healthy volunteers or | or |
| | Decrease in patients compared | 1 |
| | to healthy volunteers | |
| Level of | Level of expression generally above 5 | 1 |
| expression | | |
| Fold Change | High fold change (log2 FC | 1 |
| | visually greater than 1.5) | |
| Sense/antisense | Same expression profile between sense and | 1 |
| consistency | antisense probes | |
| Difference | Difference between DR+ and | 1 |
| visible from D1 | low DR− patients visible | |
| | from D1 (even if the difference is small). | |
| Variability | Penalty if the variability is too high. | −1 |
| SCORE | | /6 |

Thanks to this mode of attributing a score, the inventors selected 7 candidates and excluded 6 candidates whose performances appeared to be insufficient. Here in Table 17, below, the 7 markers identified with their respective score.

TABLE 17

| | NAME OF THE | | |
|---|---|---|---|
| SEQ ID | PROBESETS OF | CHROMOSOMAL | |
| NO: | THE HERV-V3 CHIP | LOCATION | SCORE |
| SEQ ID | 190665001-HERV0376 | chr19:54891074-54891496 | 6 |
| NO: 1 | | | |
| SEQ ID | 190665002-HERV0376 | chr19:54891074-54891496 | 6 |
| NO: 1 | | | |
| SEQ ID | 060281701-MALR1043 | chr6:18403673-18404108 | 5 |
| NO: 22 | | | |
| SEQ ID | 043166601-MALR1018 | chr4:184850413-184850785 | 5 |
| NO: 23 | | | |
| SEQ ID | 100090601-HERV0429 | chr10:5856198-5856795 | 4 |
| NO: 24 | | | |
| SEQ ID | 061529601-HERV0492 | chr6:107800650-107801138 | 3 |
| NO: 25 | | | |
| SEQ ID | 100871501-MALR1020 | chr10:60410534-60411224 | 3 |
| NO: 26 | | | |
| SEQ ID | 170842002-MALR1003 | chr17:78345106-78345577 | 3 |
| NO: 27 | | | |

This strategy for identifying inflammation marker made it possible to identify 6 new sequences (SEQ ID NO: 22 to 27) and to find a sequence already identified in Example 1 (SEQ ID NO: 1). It should also be noted that 2 sets of probes (probesets) target the same HERV sequence (SEQ ID NO: 1).

### EXAMPLE 7: SIGNATURE OF IMMUNE STATUS MARKERS

For this example, the aim was to determine a signature of markers making it possible to best discriminate between patients considered to be immunodepressed and those considered to be immunocompetent. To do this, the inventors used the data generated by the analyses of the HERV-V3 chips from the samples of Examples 2 and 3.

The inventors selected the HERV sequences which were differentially expressed on D3 between the patients considered to be immunodepressed (DR−) and those considered to be immunocompetent (DR+). This selection allowed identifying a list of 193 HERV sequences.

In order to obtain a reduced signature still making it possible to best discriminate between patients considered to be immunodepressed and those considered to be immunocompetent from these 193 HERV sequences, the inventors applied the «Random Forests» method (Tin Kam Ho, Random Decision Forests, AT&T Bell Laboratories). This method makes it possible to classify each sequence according to its discriminating power between the two studied conditions (immunosuppressed and immunocompetent).

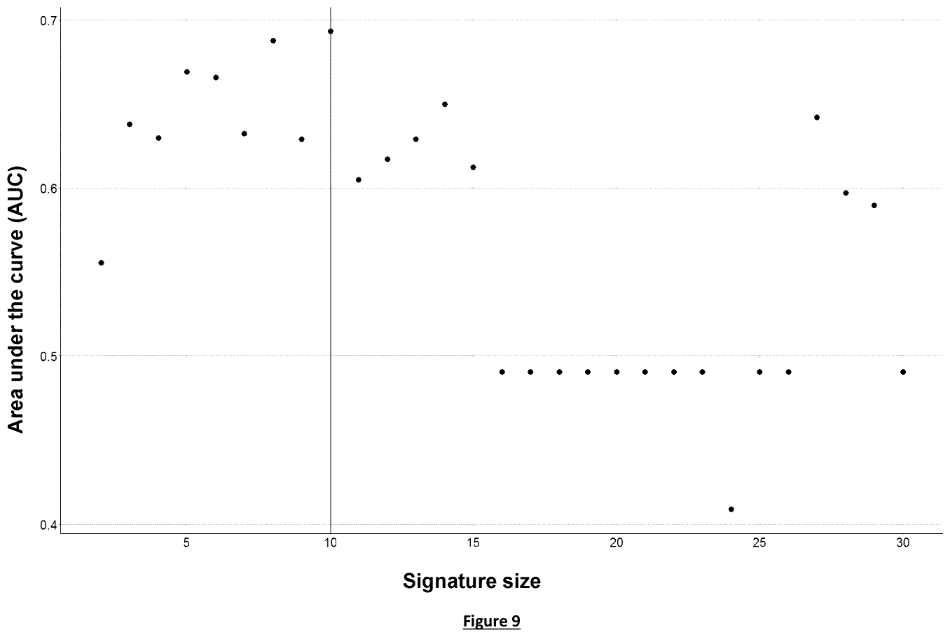

Then, to identify the optimal number of sequences in the signature, the inventors calculated the prediction performances on the cohort of example 3, by using the discriminating power for signatures of size ranging from 2 to 30 markers between the patients having a low CD74 ratio between D3 and D1 and those with this same high ratio. Thus they determined that the signature of a size of 10 sequences had the best performances (best area under the curve: AUC) (cf. FIG. 9).

In Table 18, below, the list of markers making up the signature making it possible to best discriminate between patients considered to be immunodepressed and patients considered to be immunocompetent.

TABLE 18

| | NAME OF THE | |
|---|---|---|
| SEQ ID | PROBESETS OF | CHROMOSOMAL |
| NO: | THE HERV-V3 CHIP | LOCATION |
| SEQ ID | 081921103-HERV0958 | chr8:125945973-125951030 |
| NO: 28 | | |
| SEQ ID | 032622601MR41sLU5p | chr3:167401329-167401866 |
| NO: 29 | | |
| SEQ ID | 220246901-HERV0889 | chr22:36147793-36148208 |
| NO: 30 | | |
| SEQ ID | 061827101-HERV0856 | chr6:127790579-127792191 |
| NO: 31 | | |
| SEQ ID | 170369402HE41env | chr17:35505737-35508365 |
| NO: 3 | | |
| SEQ ID | 170828901-HERV0770 | chr17:77462942-77463350 |
| NO: 32 | | |
| SEQ ID | 081921101-HERV0958 | chr8:125945973-125951030 |
| NO: 28 | | |
| SEQ ID | 081921102-HERV0958 | chr8:125945973-125951030 |
| NO: 28 | | |
| SEQ ID | 190148802-MALR1127 | chr19:14612123-14612747 |
| NO: 33 | | |
| SEQ ID | 120093401-HERV1034 | chr12:9038254-9038598 |
| NO: 34 | | |

It will be noted that this signature is composed of 8 sequences using 10 different sets of probes. Indeed, 3 sets of probes target the same sequence (SEQ ID NO: 28). In addition, in this signature we find the sequence SEQ ID NO: 3 already identified in Example 2. The HERV sequences appear in the table above in an order reflecting their «importance» in the stratification of patients according to their immune status, based on a score attributed to them by the Random forests classification algorithm.

1. A kit comprising means for amplifying and/or detecting at least one sequence selected from the sequences identified in SEQ NOS: 1 to 34 or from the sequences which have at least 99% identity with one of the identified sequences in SEQ NOS: 1 to 34, wherein the amplification and/or detection means allow the amplification and/or the detection of at most 100 biomarkers, in total.

2. The kit according to claim 1, wherein:
the amplification means comprise at least one pair of primers consisting of two amplification primers selected from amplification primers comprising, or consisting of, a nucleotide sequence complementary to at least part of a sequence selected from the sequences identified in SEQ NOS: 1 to 34 or from the sequences which exhibit at least 99% identity with one of the sequences identified in SEQ NOS: 1 to 34, and the complementary sequences of these sequences, the at least one pair of amplification primers making it possible to amplify, at least part of a sequence selected from the sequences identified in SEQ NOS: 1 to 34 or from the sequences which exhibit at least 99% of identity with one of the sequences identified in SEQ NOS: 1 to 34, and the sequences complementary to these sequences, and/or
the detection means comprise at least one hybridization probe whose nucleotide sequence comprises, or consists of, a nucleotide sequence complementary to at least part of a sequence selected from the sequences identified in SEQ NOS: 1 to 34 or from the sequences which exhibit at least 99% identity with one of the sequences identified in SEQ NOS: 1 to 34, and the sequences complementary to these sequences.

3. The kit according to claim 1, wherein at least one pair of amplification primers is selected from the following pairs of amplification primers:

| Pair of | |
|---|---|
| primers | Primers # (SEQ ID NOS, nucleotide |
| # | sequence) |
| 1 | Forward : Primer #1A |
| | (SEQ ID NO: 35, TGTACAAAACTCAAATGGTCTTC) |
| | Reverse : Primer #1B |
| | (SEQ ID NO: 36, ATGACCAACTTAGATTTCCTTGA) |
| 2 | Forward : Primer #2A |
| | (SEQ ID NO: 37, GCCAGAGAGGCATAATGAAGCA) |
| | Reverse : Primer #2B |
| | (SEQ ID NO: 38, GATTCTAAGCCTCCCCCTCATTT) |
| 3 | Forward : Primer #3A |
| | (SEQ ID NO: 39, TGGCTCATAGGGATTCCAGACT) |
| | Reverse : Primer #3B |
| | (SEQ ID NO: 40, AGCAAGTTGTCAAGAGCCAATCT) |
| 4 | Forward : Primer #4A |
| | (SEQ ID NO: 41, CACTCTAGGAATCTTAGGCA) |
| | Reverse : Primer #4B |
| | (SEQ ID NO: 42, TGAAACCAATAGTCCAGTG) |
| 5 | Forward : Primer #5A |
| | (SEQ ID NO: 43, TTCTACTGTTCACTGCTATCCTCC) |
| | Reverse : Primer #5B |
| | (SEQ ID NO: 44, CCTGTGGCAGCTTTTTGAAGTAA) |
| 6 | Forward : Primer #6A |
| | (SEQ ID NO: 45, AGAGCAGAAGAAGATGGATACT) |
| | Reverse : Primer #6B |
| | (SEQ ID NO: 46, CATGAGCTGACATCATCCAAT) |
| 7 | Forward : Primer #7A |
| | (SEQ ID NO: 47, TCTGTACTGGTTGCCCCAAC) |
| | Reverse : Primer #7B |
| | (SEQ ID NO: 48, CGTGCCAGGCCTCTAATACTTTT) |
| 8 | Forward : Primer #8A |
| | (SEQ ID NO: 49, AGGGAAGACCCCAAGATGATG) |
| | Reverse : Primer #8B |
| | (SEQ ID NO: 50, CATGCAAAGTCCAACGAGAGG) |
| 9 | Forward : Primer #9A |
| | (SEQ ID NO: 51, GGGTGGCTGCATCCTATGG) |
| | Reverse : Primer #9B |
| | (SEQ ID NO: 52, CTGGTCAGGAAAAAATTTGCCTTC) |
| 10 | Forward : Primer #10A |
| | (SEQ ID NO: 53, ACATGACATTGTCTGAACTTTGGG) |
| | Reverse : Primer #10B |
| | (SEQ ID NO: 54, TAGGACCATGCAGATACTAGTGAC) |
| 11 | Forward : Primer #11A |
| | (SEQ ID NO: 55, GAACTCCACAAACCTTGA) |
| | Reverse : Primer #11B |
| | (SEQ ID NO: 56, GCTAGAAGCTTTGGATATCT) |
| 12 | Forward : Primer #12A |
| | (SEQ ID NO: 57, TGGCTGTTACAACTTTCATG) |
| | Reverse : Primer #12B |
| | (SEQ ID NO: 58, TCTCCCTATTCTGAGCACA) |

4. The kit according to claim 1, wherein at least one hybridization probe is selected from the hybridization probes of sequences SEQ ID NOS: 59 to 274.

5. The kit according to claim 1, further comprising means for amplifying and/or detecting other biomarkers.
